(19) United States
(12) Patent Application Publication
(10) Pub. No.: US 2025/0206708 A1
(43) Pub. Date: Jun. 26, 2025

(54) AN OXAZIRIDINE PLATFORM FOR TARGETING FUNCTIONAL ALLOSTERIC METHIONINE SITES

(71) Applicants: Novartis AG, Basel (CH); THE REGENTS OF THE UNIVERSITY OF CALIFORNIA, Oakland, CA (US)

(72) Inventors: F. Dean Toste, Berkeley, CA (US); Christopher J. Chang, Berkeley, CA (US); Audrey Reeves, Berkeley, CA (US); Angel Gonzalez-valero, Berkeley, CA (US); Patrick J. Moon, Berkeley, CA (US); Edward Miller, Berkeley, CA (US); Richard Alan Lewis, Baselland (CH); Yipin Lu, Dublin, CA (US); Jeffrey M. McKenna, Carlisle, MA (US)

(21) Appl. No.: 18/852,247

(22) PCT Filed: Mar. 28, 2023
(86) PCT No.: PCT/US2023/016635

§ 371 Date: Sep. 27, 2024

Related U.S. Application Data
(60) Provisional application No. 63/325,124, filed on Mar. 29, 2022.

(51) Int. Cl. C07D 273/01; A61K 31/396; C07C 381/10; C12Q 1/48

(52) U.S. Cl. CPC C07D 273/01; A61K 31/396; C07C 381/10; C12Q 1/485

(57) ABSTRACT

The present disclosure features compounds (e.g., oxaziridine-based compounds), as well as related compositions and methods of use thereof, e.g., for selectively labeling a methionine residue in a target peptide or protein.

### CLAIM OF PRIORITY

This application claims priority to U.S. Provisional Application No. 63/325,124, filed on Mar. 29, 2022, the contents of which is incorporated herein by reference in its entirety.

This invention was made with government support under National Institutes of Health, grant numbers GM066698, GM118190, and GM139245. The government has certain rights in the invention.

### BACKGROUND

The use of covalent small molecules as reactive probes to target specific amino acid residues is key for determining new protein function as well as accelerating drug discovery. The majority of these reactive probes target cysteine and lysine. The ability to target other amino acid residues, such as methionine, would assist in tackling the vast undruggable space. As such, there is a need in the art for new compounds and methods that achieve this goal.

### SUMMARY

Described herein are methods and compositions for targeting functional allosteric methionine sites in peptides and proteins. In one aspect, the present disclosure features compounds, compositions, and related methods for covalently labeling a methionine residue within a target peptide or target protein. In some embodiments, the methods described herein provide an activity-based protein profiling (ABPP) method for profiling a target protein, specifically a target protein comprising a methionine residue. In some embodiments, the compounds comprise an oxaziridine moiety. In some embodiments, the oxaziridine moiety is capable of promoting a selective nitrene fragment transfer reactivity that is isoelectronic to native methionine oxidation by oxygen atom transfer. In an embodiment, the target protein is a kinase (e.g., a cyclin-dependent kinase). In an embodiment, the target protein is cyclin-dependent kinase 4 (CDK4).

Described compounds for targeting methionine residues in a target peptide or target protein. In one aspect, the present disclosure features a compound (e.g., an N-transfer oxidant compound) of Formula (I):

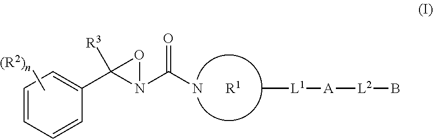

or a pharmaceutically acceptable salt, hydrate, solvate, prodrug, stereoisomer, or tautomer thereof, wherein R¹ is an heterocyclyl or heteroaryl, each of which is optionally substituted with one or more R⁴; R² is C₁-C₆ alkyl, C₂-C₆ alkenyl, C₂-C₆ alkynyl, C₁-C₆ heteroalkyl, C₁-C₆ haloalkyl, halo, cyano, or —OR^{A}; R³ is hydrogen, C₁-C₆ alkyl or halo; each of L¹ and L² is independently absent, C₁-C₆ alkylene, or C₁-C₆ heteroalkylene; A is absent, cycloalkyl, heterocyclyl, aryl, or heteroaryl, each of which is optionally substituted with one or more R⁵; B is absent, cycloalkyl, heterocyclyl, aryl, or heteroaryl, each of which is optionally substituted with one or more R⁵; each of R⁴ and R⁵ is independently C₁-C₆ alkyl, C₂-C₆ alkenyl, C₂-C₆ alkynyl, halo, cyano, or —OR^{A}, or wherein two of R⁴ or two of R⁵ may come together to form a ring with R¹, A, or B respectively; R^{A} is hydrogen, C₁-C₆ alkyl, C₂-C₆ alkenyl, C₂-C₆ alkynyl, C₁-C₆ heteroalkyl, C₁-C₆ haloalkyl, cycloalkyl, or heterocyclyl; and n is 0, 1, 2, 3, 4, or 5; provided that if both L¹ and A are absent, then L² and B are absent.

In some embodiments, R¹ is heterocyclyl, optionally substituted with one or more R⁴. In some embodiments, R¹ is a four-membered heterocyclyl, optionally substituted with one or more R⁴. In some embodiments, R¹ is a five-membered heterocyclyl, optionally substituted with one or more R⁴. In some embodiments, R¹ is a six-membered heterocyclyl, optionally substituted with one or more R⁴. In some embodiments, R¹ is a seven-membered heterocyclyl, optionally substituted with one or more R⁴. In some embodiments, R¹ is a eight-membered heterocyclyl, optionally substituted with one or more R⁴. In some embodiments, R¹ is a nine-membered heterocyclyl, optionally substituted with one or more R⁴. In some embodiments, R¹ is a ten-membered heterocyclyl, optionally substituted with one or more R⁴. In some embodiments, R¹ is a moncyclic heterocyclyl, optionally substituted with one or more R⁴. In some embodiments, R¹ is a bicyclic heterocyclyl, optionally substituted with one or more R⁴.

In some embodiments, R² is hydrogen. In some embodiments, R² is halo. In some embodiments, R² is C₁-C₆ alkyl. In some embodiments, R³ is hydrogen. In some embodiments, R³ is halo. In some embodiments, R³ is C₁-C₆ alkyl.

In some embodiments, one of L¹ and L² is independently absent. In some embodiments, one of L¹ and L² is independently C₁-C₆ alkylene. In some embodiments, one of L¹ and L² is independently C₁-C₆ heteroalkylene. In some embodiments, L¹ is absent. In some embodiments, L² is absent. In some embodiments, L¹ is C₁-C₆ alkylene. In some embodiments, L² is C₁-C₆ alkylene. In some embodiments, L¹ is C₁-C₆ heteroalkylene. In some embodiments, L² is C₁-C₆ heteroalkylene. In some embodiments, one of L¹ and L² is independently C₁-C₆ alkylene. In some embodiments, one of L¹ and L² is independently C₁-C₆ heteroalkylene. In some embodiments, each of L¹ and L² is independently absent. In some embodiments, each of L¹ and L² is independently C₁-C₆ alkylene. In some embodiments, each of L¹ and L² is independently C₁-C₆ heteroalkylene.

In some embodiments, A is absent. In some embodiments, A is aryl, optionally substituted with one or more R⁵. In some embodiments, A is heteroaryl, optionally substituted with one or more R⁵. In some embodiments, A is a nitrogen-containing heteroaryl, optionally substituted with one or more R⁵. In some embodiments, A is heterocyclyl, optionally substituted with one or more R⁵. In some embodiments, A is a nitrogen-containing heterocyclyl, optionally substituted with one or more R⁵. In some embodiments, A is an oxygen-containing heterocyclyl, optionally substituted with one or more R⁵.

In some embodiments, B is absent. In some embodiments, B is aryl, optionally substituted with one or more R⁵. In some embodiments, B is heteroaryl, optionally substituted with one or more R⁵. In some embodiments, B is a nitrogen-containing heteroaryl, optionally substituted with one or more R⁵. In some embodiments, B is heterocyclyl, optionally substituted with one or more R⁵. In some embodiments, B is a nitrogen-containing heterocyclyl, optionally substituted with one or more R⁵. In some embodiments, B is an oxygen-containing heterocyclyl, optionally substituted with one or more R⁵.

In some embodiments, the compound (e.g., an N-transfer oxidant compound) of Formula (I) is a compound of Formula (I-a):

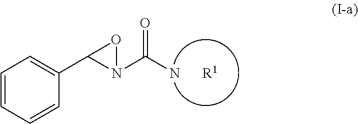

or a pharmaceutically acceptable salt, hydrate, solvate, prodrug, stereoisomer, or tautomer thereof, wherein R¹ is an optionally substituted 5- to 14-membered heteropolycycle. In some embodiments, R¹ is an optionally substituted spirocycle, fused heterocycle, bridged heterocycle, or combination thereof. In some embodiments, R¹ comprises a 4-, 5-, 6- or 7-membered first ring fused, bridged or linked by one or more common atoms to a second ring; In some embodiments, the first ring is saturated and comprises 0, 1 or 2 heteroatoms (e.g. N or O) in addition to the N shown (e.g. azetidinyl, pyrrolidinyl, pipiridinyl, azepanyl, diazinanyl, morpholinyl). In some embodiments, the second ring is 3-, 4-, 5- or 6-membered, saturated or unsaturated, optionally comprising 1-3 heteroatoms (e.g. N or O). In some embodiments, the compound has a structure of any one of Tables 1, 2, and 3. In some embodiments, the compound is an N-transfer oxidant compound of Table 1. In some embodiments, the compound is an N-transfer oxidant compound of Table 2. In some embodiments, the compound is an N-transfer oxidant compound of Table 3.

The details of one or more embodiments of the invention are set forth herein. Other features, objects, and advantages of the invention will be apparent from the Detailed Description, the Figures, the Examples, and the Claims.

### BRIEF DESCRIPTION OF THE DRAWINGS

FIGS. 1A-C shows the activity-based protein profiling (ABPP) method using oxaziridine probes for Redox-Activated Chemical Tagging (ReACT) to identify new reactive, ligandable methionine sites on CDK4. FIG. 1A shows the structures of Compounds 301, 302, and 303. FIG. 1B shows the ribbon diagram of CDK4 (2W9Z). Methionine residues modified by oxaziridines are highlighted, with colored squares representing correspondent ReACT reagents found to modify each site. Each residue is additionally labeled with its calculated solvent accessibility. FIG. 1C shows the shotgun proteomics general workflow. Isolated protein is first incubated with compound. After excess compound is neutralized, protein is tryptic-digested and analyzed via MS/MS to reveal compound site(s) of modification.

FIGS. 2A-C shows the MS/MS of Compounds 303 and 302 modifications on CDK4. All samples represent 50 μM probe treatment in 10 μg CDK4/CCND1 spiked into 90 μg mouse liver lysate. FIG. 2A shows the MS/MS of Compound 302 modification at Met264 of CDK4. FIG. 2B shows the MS/MS of the Compound 303 modification at Met169. FIG. 2C shows the MS/MS of Met264 of CDK4.

FIGS. 3A-C shows the clustering analyses of the compounds from Example 8. FIG. 3A shows the clustering analysis in terms of mol Log P vs Frequency. FIG. 3B shows the clustering analysis in terms of molecular weight vs Frequency. FIG. 3C shows the clustering analysis in terms of the number of rotational bonds vs Frequency.

FIGS. 4A-B contains graphs comparing automatically calculated vs manually obtained values. FIG. 4A is a graph showing the comparison of νC═O obtained from automated vs. manual workflows and FIG. 4B is a recreated kinetic model using the automatically calculated νC═O with predicted hydrolysis rates of select sulfimines (data points in blue).

FIGS. 5A-E shows the design, synthesis, and evaluation of the oxaziridine-based covalent ligand library for targeting methionine sites and identification of Compound 148 as a covalent modifier of CDK4 via gel-based ABPP screening. FIG. 5A shows the design of oxaziridine fragments that favor formation of N-transfer sulfimine over O-transfer sulfoxide products upon reaction with methionine using principle component analysis (PCA). Rates of sulfimine hydrolysis correlate with calculated νC═O values. FIG. 5B shows the schematic of gel-based ABPP screening workflow. The protein target is preincubated with covalent ligand followed by treatment with Compound 303. Excess oxaziridine is then quenched with N-acetyl methionine (NAM) and the sample treated with DBCO-Cy3 overnight for fluorescence detection. Samples are finally separated by SDS-PAGE. Loss of fluorescent signal suggests competitive ligand binding to the protein. Silver stain is used to identify and exclude samples where signal loss corresponds to overall protein loss, likely due to aggregation induced by the ligand. FIG. 5C shows the representative structure types within the oxaziridine fragment library, organized by common functional groups. FIG. 5D shows representative data from gel-based ABPP screen. CDK4 and ligand incubated at equimolar doses. FIG. 5E shows the structure of Compound 148 fragment identified in gel-based ABPP screens as a competitive ligand for CDK4. Dose-dependent treatment of Compound 148 against CDK4, CDK1, and CDK6 shows selective loss of fluorescent signal only with CDK4, suggesting isoform-specific engagement of this target.

FIGS. 6A-H shows the oxaziridine library screen against CDK4-CCND1. Samples were treated according to gel-ABPP method outlined in Example 3 at 50 nM compound and 50 nM CDK4-CCND1.

FIGS. 7A-B shows the aggregation test with Compound 148. Lysates were treated with varying doses of Compound 148 according to gel-ABPP method outlined in Example x. FIG. 7A shows the Cy3 channel image from the aggregation test. FIG. 7B shows the silver stain image from the aggregation test.

FIGS. 8A-B shows that Compound 148 is a covalent modifier of CDK4 at M169 and inhibits its activity on purified protein. FIG. 8A shows the MS/MS spectrum of Compound 148-modified CDK4 showing functionalization at M169. FIG. 8B shows the activity assay on purified CDK4 protein showing dose-dependent inhibition in response to Compound 148 treatment.

FIG. 9 shows an additional MS/MS spectrum. MS/MS of CDK4, modified with Compound 148 at M264. Isolated CDK4-CCND1 spiked in mouse liver lysate was treated with Compound 148 as shotgun proteomics method outlined in Example 5.

FIGS. 10A-G shows a decrease in cell viability and inhibition of cellular CDK4 activity by Compound 148 and Compound 300, in various cancer cell models. FIG. 10A shows the treatment of ribociclib-sensitive cell lines with 500 μM Compound 148, demonstrating its ability to decrease cell viability. The MCF-7 line displays the highest sensitivity across the lines screened. Error bars represent standard deviation of at least three biological replicates. FIG. 10B shows a decrease in cell viability of MCF7 cells to increasing doses of Compound 148. Error bars represent standard deviation of at least three biological replicates. FIG. 10D shows a schematic of how measurements of cellular CDK4 activity were assessed, using Rb as a native CDK4 substrate. Western blot analysis of the extent of Rb phosphorylation provides a method for product detection. FIG. 10E shows the MS/MS data showing that Compound 300 is a covalent modifier of CDK4 at the same M169 site as the parent Compound 148 fragment. FIG. 10F shows the Western blots assessing changes in cellular CDK4 activity with increasing added Compound 148 and Compound 300 concentrations. Treatments of MCF-7 cells with Compound 148 and Compound 300 result in lower signals at Rb sites phosphorylated by active CDK4. FIG. 10G shows the competition binding assay between Compound 148 and Compound 300 for CDK4, providing evidence for target engagement in cells. MCF-7 lysates with CDK4-FLAG plasmid overexpression were pretreated with varying concentrations of Compound 148 as indicated, followed by a 500 μM treatment of Compound 300. All samples were then subjected to Copper-catalyzed azide-alkyne cycloaddition (CuAAC) to DTB-N3 and pulled down onto high-capacity streptavidin agarose beads. Supernatant was collected, and bound beads eluted with 50% MeCN/0.1% FA. Eluted sample was lyophilized, reconstituted in PBS, and separated via SDS-PAGE. Gel was transferred to PVDF membrane, and CDK4 signal was assessed via western blot using anti-CDK4 antibody.

FIG. 11 shows the competition of Compound 148 and Compound 300 by gel. Lysate was collected from MCF-7 cells overexpressed with CDK4 and treated with DMSO or Compound 148 as indicated. Then, all samples were treated with 500 μM Compound 300, followed by a click step to DTB-N3 and subsequent pulldown onto high-capacity streptavidin agarose beads overnight at 4° C. Supernatant was saved and run as “S” lanes, proteins bound to resin were eluted and run as “E” lanes. Samples were separated by SDS-PAGE and bands visualized by Coomassie staining. CDK4 appears at 36 kDa.

FIGS. 12A-B shows the Reactivity comparison of Compound 302 and Compound 300. Lysate was collected from MCF-7 cells overexpressed with CDK4 and treated with either 10 μM Compound 302 or 10 μM Compound 300. Then, Alexa488-azide was appended to tagged proteins via copper-catalyzed azide-alkyne cycloaddition (CuAAC). Samples were boiled, separated by SDS-PAGE, and visualized.

FIG. 13 shows the ReACT covalent ligand probe platform that enabled discovery of a reciprocal oxidation/phosphorylation crosstalk pathway in CDK4 through proximal allosteric M169 and T172 sites, where M169-targeted oxidation can inhibit CDK4 activity by preventing phosphorylation at T172. FIG. 13A shows a schematic outlining oxidation/phosphorylation crosstalk between CDK4 M169 and T172. Under low oxidative conditions, T172 of CDK4 is phosphorylated by cyclin-dependent activating kinase (CAK) as part of a critical activating step leading to cell division to pass the S-phase checkpoint. High oxidative conditions can lead to oxidation at M169, which blocks the T172 phosphorylation site, thus preventing cell division via S-phase checkpoint failure. This crosstalk identifies a methionine redox-dependent vulnerability for potential therapeutic intervention. FIG. 13B shows the ribbon structure (2W9Z) highlighting proximity of M169 and T172. FIG. 13C shows the monitoring CDK4 phosphorylation status using 2D-western blot analyses with phospho-specific CDK4 antibodies. Phosphorylation at T172 decreases with increasing concentrations of added Compound 148 in MCF-7 cells. Spots 1, 2, 3, and 4 represent different phosphorylation states of CDK4, with spot 1 being unphosphorylated and spot 3 being monophosphorylated at T172. Peaks represent changes in intensity of spots normalized to peak 1 intensity. A clear decrease in spot 3 is observed upon treatment with increasing doses of Compound 148, consistent with a model where this covalent ligand inhibits CDK4 activity by promoting M169 oxidation to block T172 phosphorylation.

### DETAILED DESCRIPTION

Covalent small molecules that target specific amino acid residues are powerful chemical tools that can reveal fundamental new protein function and identify lead candidates for accelerating drug discovery. Indeed, led by advances across broad fields encompassing organic chemistry, chemical biology, cell biology, and bioinformatics, covalent therapeutics now constitute approximately 30% of enzyme-targeting FDA-approved drugs. In this context, activity-based protein profiling (ABPP), where chemical probes measure protein function rather than protein abundance, has enabled new modalities for fragment-based drug discovery by applying small-molecule screening efforts in conjunction with chemoproteomics for target and site identification and characterization. These technologies rely on residue-specific covalent warheads that can be used from proteins to proteomes, yet the majority of reactive probe development to tackle this vast undruggable space has targeted cysteine or lysine, with relatively limited expansion of this chemical toolbox to other nucleophilic residues like tyrosine and glutamate/aspartate.

Motivated to contribute to this area and use activity-based chemical probes for biological applications, the inventors have developed bioconjugation methods for methionine, one of two privileged sulfur-containing amino acids along with cysteine. Methionine is distinguished by its characteristic thioether moiety, which endows this hydrophobic amino acid with high redox activity and low nucleophilicity relative to its highly redox-active and nucleophilic cysteine congener. The methionine sulfur atom enables not only greater rotational freedom through lower strain gauche interactions but also provides the opportunity for unique single-atom post-translational modifications (PTM) through a reversible two-electron oxidation to generate both (R) and (S)-methionine sulfoxide products. Akin to kinase writer and phosphatase eraser pairs for installing and removing phosphate PTMs, respectively, stereospecific reduction of methionine sulfoxide sites catalyzed by methionine sulfoxide reductase (Msr) eraser proteins can remove these single-oxygen PTMs. This reversible methionine thioether/sulfoxide cycle plays an integral part in the redox regulation of cell signaling events, antioxidant function, and other forms of protein regulation.

In contrast to cysteine and other protein nucleophiles, the reactivity of protein-bound methionine more often dictated by its redox potential rather than by its pKa. As such, the inventors developed Redox-Activated Chemical Tagging (ReACT), described herein, a versatile bioconjugation method that targets methionine. Without being bound by theory, the ReACT method entails the use of oxaziridine reagents that promote selective nitrene fragment transfer reactivity that is isoelectronic to native methionine oxidation by oxygen atom transfer. Methionine functionalization with ReACT may proceed selectively and rapidly at physiological pH and can generate stable, mass-spectrometry compatible sulfimine adducts, enabling further chemoproteomic characterization of putative protein targets and sites of modification. Indeed, ReACT has found utility in the context of synthesis of stapled cyclic peptides, production of antibody-drug conjugates (MetMAb), proximity-activated imaging reporters for protein function (PAIR), ¹⁸F radioimaging tracers and probes for protein and nucleic acid biotinylation (BioReACT).

The present disclosure features compounds, compositions, and related methods for targeting methionine residues in a target peptide or a target protein, e.g., outlining the compounds and methods useful in the ReACT system. In some embodiments, the compounds described herein are capable of covalently labeling a methionine residue within a target peptide or target protein. In an embodiment, the target protein is a kinase (e.g., a cyclin-dependent kinase). In an embodiment, the target protein is cyclin-dependent kinase 4 (CDK4), a serine/threonine kinase which is shown to play a role as a master regulator of mitogenic signaling responsible for G1-S phase progression of the cell cycle, is highlighted. CDK4 is a high-value therapeutic target that is commonly misregulated in a variety of cancers and is one of many CDKs targeted in cancer drug therapy efforts.

Further details regarding the compounds, compositions, and related methods are contained herein.

### Selected Definitions

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

Unless contraindicated or noted otherwise, in these descriptions and throughout this specification, the terms “a” and “an” mean one or more (e.g., to at least one), the term “or” means and/or.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

The term “alkyl” refers to a hydrocarbon group selected from linear and branched saturated hydrocarbon groups of 1-18 (“C₁-C₁₈”), or 1-12 (“C₁-C₁₂”), or 1-6 (“C₁-C₆”) carbon atoms. Examples of the alkyl group include methyl (C₁), ethyl (C₂), 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of the alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups. Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁_C₁₀ alkyl (e.g., —CH₃). In certain embodiments, the alkyl group is substituted C₁-C₆ alkyl.

Lower alkyl as used herein refers to a radical of 1-8 (“C₁-C₈”) carbon atoms, preferably 1-6 (“C₁-C₆”), more preferably 1-4 (“C₁-C₄”) carbon atoms; lower alkenyl or alkynyl means 2-8 (“C₂-C₈”), 2-6 (“C₂-C₆”) or 2-4 (“C₂-C₄”) carbon atoms.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₉), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

The term “cycloalkyl” herein refers to a hydrocarbon group selected from saturated and partially unsaturated cyclic hydrocarbon groups, comprising monocyclic and polycyclic (e.g., bicyclic and tricyclic) groups. For example, the cycloalkyl group may be of 3-12 (“C₃-C₁₂”), or 3-8 (“C₃-C₈”), or 3-6 (“C₃-C₆”) carbon atoms. Even further for example, the cycloalkyl group may be a monocyclic group of 3-12 (“C₃-C₁₂”), or 3-8 (“C₃-C₈”), or 3-6 (“C₃-C₆”) carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e. partially unsaturated), but is not fully conjugated, and is not aromatic, as aromatic is defined herein.

The term “aryl” herein refers to a group selected from: 5- and 6-membered carbocyclic aromatic rings, for example, phenyl; bicyclic ring systems such as 7-12 membered bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group is selected from 5- and 6-membered carbocyclic aromatic rings fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap with heteroaryl, separately defined below. Hence, if one or more carbocyclic aromatic rings are fused with a heterocyclic aromatic ring, the resulting ring system is heteroaryl, not aryl, as defined herein.

The term “halogen” or “halo” refers to F, Cl, Br or I.

The term “heteroalkyl” refers to an alkyl group comprising at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

The term “heteroaryl” refers to a group selected from: 5- to 7-membered aromatic, monocyclic rings comprising 1, 2, 3 or 4 heteroatoms selected from N, O, and S, with the remaining ring atoms being carbon; 8- to 12-membered bicyclic rings comprising 1, 2, 3 or 4 heteroatoms, selected from N, O, and S, with the remaining ring atoms being carbon and wherein at least one ring is aromatic and at least one heteroatom is present in the aromatic ring; and 11- to 14-membered tricyclic rings comprising 1, 2, 3 or 4 heteroatoms, selected from N, O, and S, with the remaining ring atoms being carbon and wherein at least one ring is aromatic and at least one heteroatom is present in an aromatic ring.

For example, the heteroaryl group includes a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

The term “heterocyclic” or “heterocycle” or “heterocyclyl” refers to a ring selected from 4- to 12-membered monocyclic, bicyclic and tricyclic, saturated and partially unsaturated rings comprising at least one carbon atoms in addition to 1, 2, 3 or 4 heteroatoms, selected from oxygen, sulfur, and nitrogen. “Heterocycle” also refers to a 5- to 7-membered heterocyclic ring comprising at least one heteroatom selected from N, O, and S fused with 5-, 6-, and/or 7-membered cycloalkyl, carbocyclic aromatic or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e. partially unsaturated). The heterocycle may be substituted with oxo. The point of the attachment may be carbon or heteroatom in the heterocyclic ring. A heterocycle is not a heteroaryl as defined herein.

Examples of the heterocycle include, but not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl and 1,1-dioxo-1-thiomorpholinyl.

“Heteropolycycle” refers to a ring system comprising a first ring, which comprises at least one heteroatom selected from N, O, and S, fused, bridged or linked by one or more common atoms to a second ring. Examples of the heteropolycycle include, but are not limited to, a spirocycle, a fused heterocycle, a bridged heterocycle, or combination thereof. The heteropolycycle may be substituted.

The terms “heterocycle”, “heterocyclyl”, and “heterocyclic” may be used interchangeably with terms heteropolycycle and heteropolycyclic.

Substituents, such as R¹-R⁸, particularly R¹, R³, R⁵-R⁸, are selected from: halogen, —R, —OR, ═O, =NR, ═N—OR, —NRR″, —SR, —SiR R″R″, —OC(O)R, —C(O)R, —CO₂R, —CONRR″, —OC(O)NRR″, —NR″C(O)R, —NR—C(O)NR″R″, —NR—SO₂NR″, —NR″CO₂R, —NH—C(NH₂)=NH, —NRC(NH₂)=NH, —NH—C(NH₂)=NR, —S(O)R, —SO₂R, —SO₂NRR″, —NR″SO₂R, —CN and —NO₂, —N₃, —CH(Ph)₂, perfluoro(C₁-C₄)alkoxy and perfluoro(C₁-C₄)alkyl, in a number ranging from zero to three, with those groups having zero, one or two substituents being particularly preferred. R, R, R″ and R″ each independently refer to hydrogen, unsubstituted (C₁-C₈)alkyl and heteroalkyl, unsubstituted aryl, aryl substituted with one to three halogens, unsubstituted alkyl, alkoxy or thioalkoxy groups, or aryl-(C₁-C₄)alkyl groups. When R and R″ are attached to the same nitrogen atom, they can be combined with the nitrogen atom to form a 5-, 6- or 7-membered ring. Hence, —NRR″ includes 1-pyrrolidinyl and 4-morpholinyl, “alkyl” includes groups such as trihaloalkyl (e.g., —CF₃ and —CH₂CF₃), and when the aryl group is 1,2,3,4-tetrahydronaphthalene, it may be substituted with a substituted or unsubstituted (C₃-C₇)spirocycloalkyl group. The (C₃-C₇)spirocycloalkyl group may be substituted in the same manner as defined herein for “cycloalkyl”.

Preferred substituents are selected from: halogen, —R, —OR, ═O, —NRR″, —SR, —SiRR″R″, —OC(O)R, —C(O)R, —CO₂R, —CONRR″, —OC(O)NRR″, —NR″C(O)R, —NR″CO₂R, —NR—SO₂NR″R″, —S(O)R, —SO₂R, —SO₂NRR″, —NR″SO₂R, —CN and —NO₂, perfluoro(C₁-C₄)alkoxy and perfluoro(C₁-C₄)alkyl, where R and R″ are as defined above.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6] and [6,6] ring systems as mentioned above; a fused bicyclic aryl ring such as 7- to 12-membered bicyclic aryl ring systems as mentioned above, a fused tricyclic aryl ring such as 10- to 15-membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The compounds may contain an asymmetric center and may thus exist as enantiomers. Where the compounds possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds and/or pharmaceutically acceptable salts thereof are intended to be included. Unless specifically mentioned otherwise, reference to one isomer applies to any of the possible isomers. Whenever the isomeric composition is unspecified, all possible isomers are included.

The compounds of the invention may also contain unnatural proportions of atomic isotopes at one or more of the atoms that constitute such compounds, such as deuterium, e.g. —CD₃, CD₂H or CDH₂ in place of methyl. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the invention.

### Compounds

Described compounds for targeting methionine residues in a target peptide or target protein. In one aspect, the present disclosure features a compound (e.g., an N-transfer oxidant compound) of Formula (I):

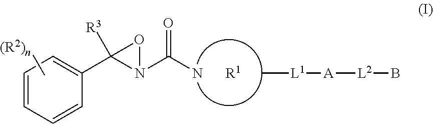

or a pharmaceutically acceptable salt, hydrate, solvate, prodrug, stereoisomer, or tautomer thereof, wherein R¹ is an heterocyclyl or heteroaryl, each of which is optionally substituted with one or more R⁴; R² is C₁-C₆ alkyl, C₂-C₆ alkenyl, C₂-C₆ alkynyl, C₁-C₆ heteroalkyl, C₁-C₆ haloalkyl, halo, cyano, or —OR^{A}; R³ is hydrogen, C₁-C₆ alkyl or halo; each of L¹ and L² is independently absent, C₁-C₆ alkylene, or C₁-C₆ heteroalkylene; A is absent, cycloalkyl, heterocyclyl, aryl, or heteroaryl, each of which is optionally substituted with one or more R⁵; B is absent, cycloalkyl, heterocyclyl, aryl, or heteroaryl, each of which is optionally substituted with one or more R⁵; each of R⁴ and R⁵ is independently C₁-C₆ alkyl, C₂-C₆ alkenyl, C₂-C₆ alkynyl, halo, cyano, or —OR^{A}, or wherein two of R⁴ or two of R⁵ may come together to form a ring with R¹, A, or B respectively; R^{A} is hydrogen, C₁-C₆ alkyl, C₂-C₆ alkenyl, C₂-C₆ alkynyl, C₁-C₆ heteroalkyl, C₁-C₆ haloalkyl, cycloalkyl, or heterocyclyl; and n is 0, 1, 2, 3, 4, or 5; provided that if both L¹ and A are absent, then L² and B are absent.

In some embodiments, R¹ is heterocyclyl, optionally substituted with one or more R⁴. In some embodiments, R¹ is a four-membered heterocyclyl, optionally substituted with one or more R⁴. In some embodiments, R¹ is a five-membered heterocyclyl, optionally substituted with one or more R⁴. In some embodiments, R¹ is a six-membered heterocyclyl, optionally substituted with one or more R⁴. In some embodiments, R¹ is a seven-membered heterocyclyl, optionally substituted with one or more R⁴. In some embodiments, R¹ is a eight-membered heterocyclyl, optionally substituted with one or more R⁴. In some embodiments, R¹ is a nine-membered heterocyclyl, optionally substituted with one or more R⁴. In some embodiments, R¹ is a ten-membered heterocyclyl, optionally substituted with one or more R⁴. In some embodiments, R¹ is a moncyclic heterocyclyl, optionally substituted with one or more R⁴. In some embodiments, R¹ is a bicyclic heterocyclyl, optionally substituted with one or more R⁴.

In some embodiments, R² is hydrogen. In some embodiments, R² is halo. In some embodiments, R² is C₁-C₆ alkyl. In some embodiments, R³ is hydrogen. In some embodiments, R³ is halo. In some embodiments, R³ is C₁-C₆ alkyl.

In some embodiments, one of L¹ and L² is independently absent. In some embodiments, one of L¹ and L² is independently C₁-C₆ alkylene. In some embodiments, one of L¹ and L² is independently C₁-C₆ heteroalkylene. In some embodiments, L¹ is absent. In some embodiments, L² is absent. In some embodiments, L¹ is C₁-C₆ alkylene. In some embodiments, L² is C₁-C₆ alkylene. In some embodiments, L¹ is C₁-C₆ heteroalkylene. In some embodiments, L² is C₁-C₆ heteroalkylene. In some embodiments, one of L¹ and L² is independently C₁-C₆ alkylene. In some embodiments, one of L¹ and L² is independently C₁-C₆ heteroalkylene. In some embodiments, each of L¹ and L² is independently absent. In some embodiments, each of L¹ and L² is independently C₁-C₆ alkylene. In some embodiments, each of L¹ and L² is independently C₁-C₆ heteroalkylene.

In some embodiments, A is absent. In some embodiments, A is aryl, optionally substituted with one or more R⁵. In some embodiments, A is heteroaryl, optionally substituted with one or more R⁵. In some embodiments, A is a nitrogen-containing heteroaryl, optionally substituted with one or more R⁵. In some embodiments, A is heterocyclyl, optionally substituted with one or more R⁵. In some embodiments, A is a nitrogen-containing heterocyclyl, optionally substituted with one or more R⁵. In some embodiments, A is an oxygen-containing heterocyclyl, optionally substituted with one or more R⁵.

In some embodiments, B is absent. In some embodiments, B is aryl, optionally substituted with one or more R⁵. In some embodiments, B is heteroaryl, optionally substituted with one or more R⁵. In some embodiments, B is a nitrogen-containing heteroaryl, optionally substituted with one or more R⁵. In some embodiments, B is heterocyclyl, optionally substituted with one or more R⁵. In some embodiments, B is a nitrogen-containing heterocyclyl, optionally substituted with one or more R⁵. In some embodiments, B is an oxygen-containing heterocyclyl, optionally substituted with one or more R⁵.

In some embodiments, the compound (e.g., an N-transfer oxidant compound) of Formula (I) is a compound of Formula (I-a):

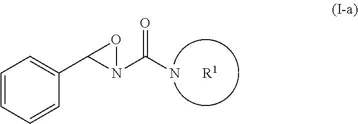

or a pharmaceutically acceptable salt, hydrate, solvate, prodrug, stereoisomer, or tautomer thereof, wherein R¹ is an optionally substituted 5- to 14-membered heteropolycycle. In some embodiments, R¹ is an optionally substituted spirocycle, fused heterocycle, bridged heterocycle, or combination thereof. In some embodiments, R¹ comprises a 4-, 5-, 6- or 7-membered first ring fused, bridged or linked by one or more common atoms to a second ring; In some embodiments, the first ring is saturated and comprises 0, 1 or 2 heteroatoms (e.g. N or O) in addition to the N shown (e.g. azetidinyl, pyrrolidinyl, pipiridinyl, azepanyl, diazinanyl, morpholinyl). In some embodiments, the second ring is 3-, 4-, 5- or 6-membered, saturated or unsaturated, optionally comprising 1-3 heteroatoms (e.g. N or O). In some embodiments, the compound has a structure provided in any one of Tables 1, 2, or 3. In some embodiments, the compound is an N-transfer oxidant compound of Table 1. In some embodiments, the compound is an N-transfer oxidant compound of Table 2. In some embodiments, the compound is an N-transfer oxidant compound of Table 3.

TABLE 1
Exemplary Compounds of Formula (I)

| Cmpd No. | Ref No. | Structure |
|---|---|---|
| 100 | 1oxA1 | 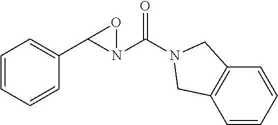 [SMILES: O=C(N1Cc2ccccc2C1)N1OC1c1ccccc1] |
| 101 | 1oxA3 | 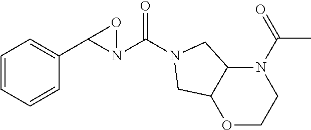 [SMILES: CC(=O)N1CCOC2CN(C(=O)N3OC3c3ccccc3)CC21] |
| 102 | 1oxA4 | 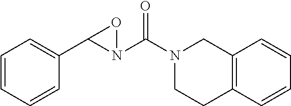 [SMILES: O=C(N1CCc2ccccc2C1)N1OC1c1ccccc1] |
| 103 | 1oxA5 | 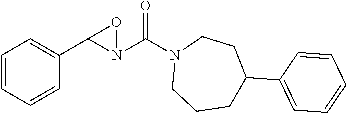 [SMILES: O=C(N1CCCC(c2ccccc2)CC1)N1OC1c1ccccc1] |
| 104 | 1oxA7 | 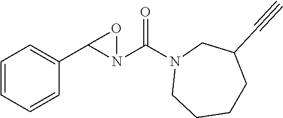 [SMILES: C#CC1CCCCN(C(=O)N2OC2c2ccccc2)C1] |
| 105 | 1oxA8 | 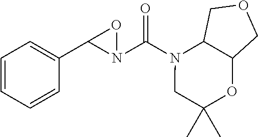 [SMILES: CC1(C)CN(C(=O)N2OC2c2ccccc2)C2COCC2O1] |
| 106 | 1oxA9 | 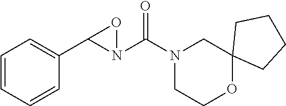 [SMILES: O=C(N1CCOC2(CCCC2)C1)N1OC1c1ccccc1] |
| 107 | 1oxA10 | 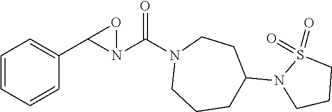 [SMILES: O=C(N1CCCC(N2CCCS2(=O)=O)CC1)N1OC1c1ccccc1] |
| 108 | 1oxA11 | 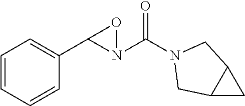 [SMILES: O=C(N1CC2CC2C1)N1OC1c1ccccc1] |
| 109 | 1oxA12 | 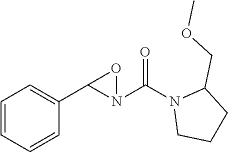 [SMILES: COCC1CCCN1C(=O)N1OC1c1ccccc1] |
| 110 | 1oxB3 | 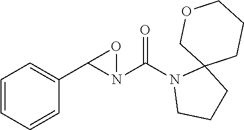 [SMILES: O=C(N1OC1c1ccccc1)N1CCCC12CCCOC2] |
| 111 | 1oxB4 | 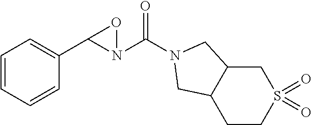 [SMILES: O=C(N1CC2CCS(=O)(=O)CC2C1)N1OC1c1ccccc1] |
| 112 | 1oxB6 | 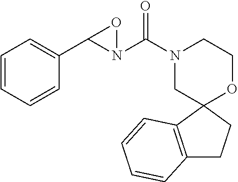 [SMILES: O=C(N1CCOC2(CCc3ccccc32)C1)N1OC1c1ccccc1] |
| 113 | 1oxB7 | 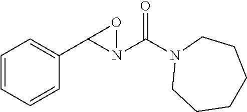 [SMILES: O=C(N1CCCCCC1)N1OC1c1ccccc1] |
| 114 | 1oxB9 | 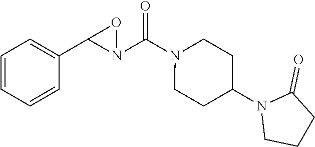 [SMILES: O=C1CCCN1C1CCN(C(=O)N2OC2c2ccccc2)CC1] |
| 115 | 1oxB11 | 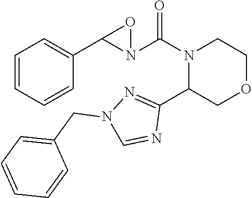 [SMILES: O=C(N1CCOCC1c1ncn(Cc2ccccc2)n1)N1OC1c1ccccc1] |
| 116 | 1oxC1 | 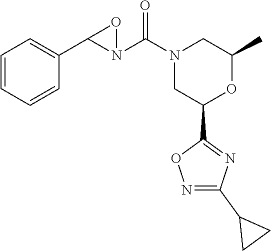 [SMILES: C[C@@H]1CN(C(=O)N2OC2c2ccccc2)C[C@H](c2nc(C3CC3)no2)O1] |
| 117 | 1oxC2 | 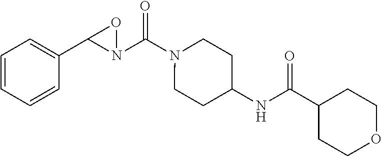 [SMILES: O=C(NC1CCN(C(=O)N2OC2c2ccccc2)CC1)C1CCOCC1] |
| 118 | 1oxC3 | 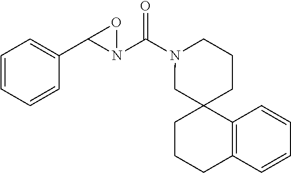 [SMILES: O=C(N1CCCC2(CCCc3ccccc32)C1)N1OC1c1ccccc1] |
| 119 | 1oxC4 | 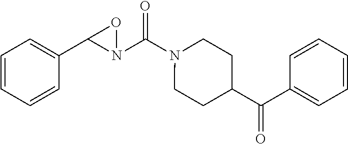 [SMILES: O=C(c1ccccc1)C1CCN(C(=O)N2OC2c2ccccc2)CC1] |
| 120 | 1oxC5 | 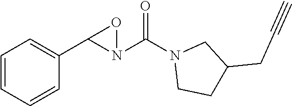 [SMILES: C#CCC1CCN(C(=O)N2OC2c2ccccc2)C1] |
| 121 | 1oxC8 | 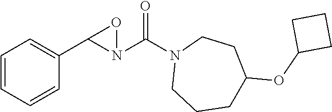 [SMILES: O=C(N1CCCC(OC2CCC2)CC1)N1OC1c1ccccc1] |
| 122 | 1oxC9 | 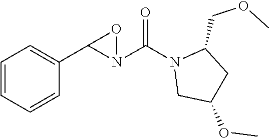 [SMILES: COC[C@@H]1C[C@H](OC)CN1C(=O)N1OC1c1ccccc1] |
| 123 | 1oxC11 | 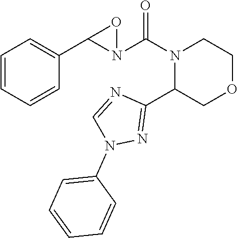 [SMILES: O=C(N1CCOCC1c1ncn(-c2ccccc2)n1)N1OC1c1ccccc1] |
| 124 | 1oxC12 | 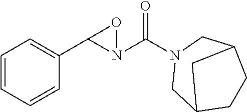 [SMILES: O=C(N1CC2CCC(C2)C1)N1OC1c1ccccc1] |
| 125 | 1oxD2 | 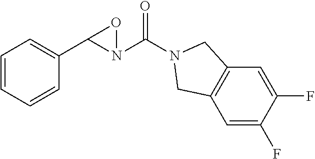 [SMILES: O=C(N1Cc2cc(F)c(F)cc2C1)N1OC1c1ccccc1] |
| 126 | 1oxD4 | 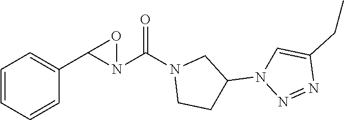 [SMILES: CCc1cn(C2CCN(C(=O)N3OC3c3ccccc3)C2)nn1] |
| 127 | 1oxD5 | 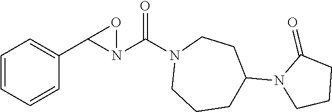 [SMILES: O=C1CCCN1C1CCCN(C(=O)N2OC2c2ccccc2)CC1] |
| 128 | 1oxD6 | 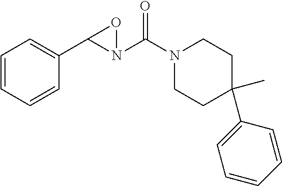 [SMILES: CC1(c2ccccc2)CCN(C(=O)N2OC2c2ccccc2)CC1] |
| 129 | 1oxD8 | 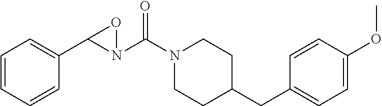 [SMILES: COc1ccc(CC2CCN(C(=O)N3OC3c3ccccc3)CC2)cc1] |
| 130 | 1oxD9 | 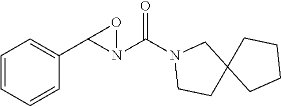 [SMILES: O=C(N1CCC2(CCCC2)C1)N1OC1c1ccccc1] |
| 131 | 1oxD10 | 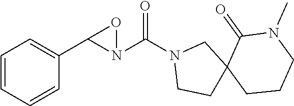 [SMILES: CN1CCCC2(CCN(C(=O)N3OC3c3ccccc3)C2)C1=O] |
| 132 | 1oxE1 | 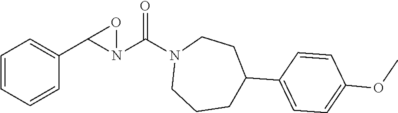 [SMILES: COc1ccc(C2CCCN(C(=O)N3OC3c3ccccc3)CC2)cc1] |
| 133 | 1oxE2 | 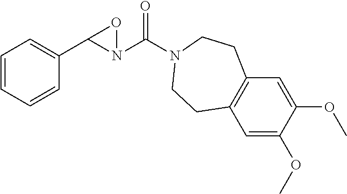 [SMILES: COc1cc2c(cc1OC)CCN(C(=O)N1OC1c1ccccc1)CC2] |
| 134 | 1oxE4 | 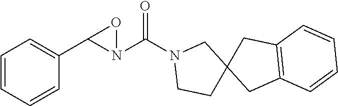 [SMILES: O=C(N1CCC2(Cc3ccccc3C2)C1)N1OC1c1ccccc1] |
| 135 | 1oxE5 | 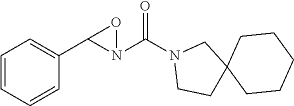 [SMILES: O=C(N1CCC2(CCCCC2)C1)N1OC1c1ccccc1] |
| 136 | 1oxE6 | 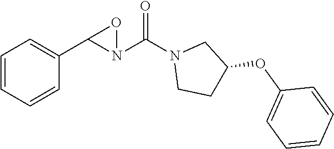 [SMILES: O=C(N1CC[C@@H](Oc2ccccc2)C1)N1OC1c1ccccc1] |
| 137 | 1oxE8 | 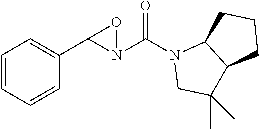 [SMILES: CC1(C)CN(C(=O)N2OC2c2ccccc2)[C@H]2CCC[C@H]21] |
| 138 | 1oxE10 | 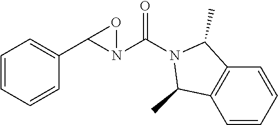 [SMILES: C[C@@H]1c2ccccc2[C@@H](C)N1C(=O)N1OC1c1ccccc1] |
| 139 | 1oxE11 | 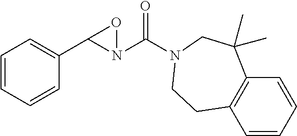 [SMILES: CC1(C)CN(C(=O)N2OC2c2ccccc2)CCc2ccccc21] |
| 140 | 1oxE12 | 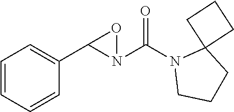 [SMILES: O=C(N1OC1c1ccccc1)N1CCCC12CCC2] |
| 141 | 1F1 | 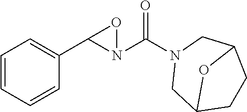 [SMILES: O=C(N1CC2CCC(C1)O2)N1OC1c1ccccc1] |
| 142 | 1F2 | 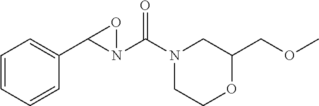 [SMILES: COCC1CN(C(=O)N2OC2c2ccccc2)CCO1] |
| 143 | 1F6 | 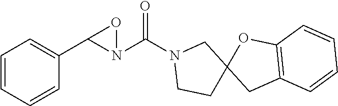 [SMILES: O=C(N1CCC2(Cc3ccccc3O2)C1)N1OC1c1ccccc1] |
| 144 | 1F7 | 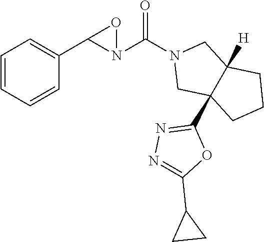 [SMILES: O=C(N1C[C@@H]2CCC[C@]2(c2nnc(C3CC3)o2)C1)N1OC1c1ccccc1] |
| 145 | 1F8 | 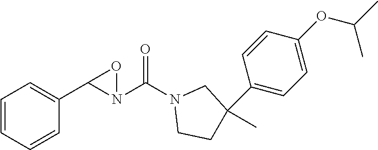 [SMILES: CC(C)Oc1ccc(C2(C)CCN(C(=O)N3OC3c3ccccc3)C2)cc1] |
| 146 | 1F9 | 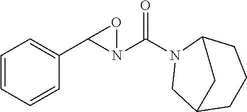 [SMILES: O=C(N1CC2CCCC1C2)N1OC1c1ccccc1] |
| 147 | 1F10 | 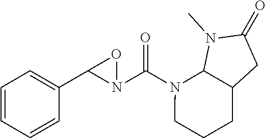 [SMILES: CN1C(=O)CC2CCCN(C(=O)N3OC3c3ccccc3)C21] |
| 148 | 1F11 | 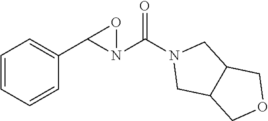 [SMILES: O=C(N1CC2COCC2C1)N1OC1c1ccccc1] |
| 149 | 1F12 | 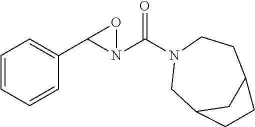 [SMILES: O=C(N1CCC2CCC(C2)C1)N1OC1c1ccccc1] |
| 150 | 1G1 | 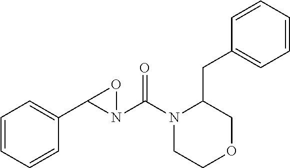 [SMILES: O=C(N1CCOCC1Cc1ccccc1)N1OC1c1ccccc1] |
| 151 | 1G2 | 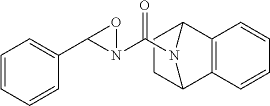 [SMILES: O=C(N1OC1c1ccccc1)N1C2CCC1c1ccccc12] |
| 152 | 1G3 | 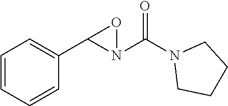 [SMILES: O=C(N1CCCC1)N1OC1c1ccccc1] |
| 153 | 1G4 | 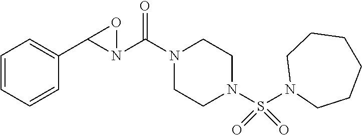 [SMILES: O=C(N1CCN(S(=O)(=O)N2CCCCCC2)CC1)N1OC1c1ccccc1] |
| 154 | 1G5 | 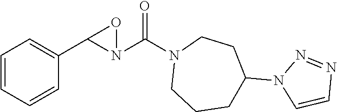 [SMILES: O=C(N1CCCC(n2ccnn2)CC1)N1OC1c1ccccc1] |
| 155 | 1G6 | 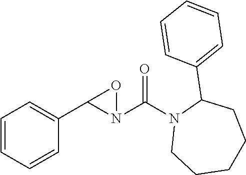 [SMILES: O=C(N1CCCCCC1c1ccccc1)N1OC1c1ccccc1] |
| 156 | 1G7 | 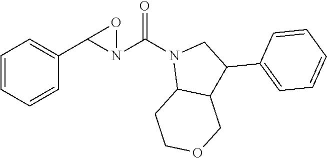 [SMILES: O=C(N1CC(c2ccccc2)C2COCCC21)N1OC1c1ccccc1] |
| 157 | 1G11 | 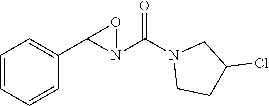 [SMILES: O=C(N1CCC(Cl)C1)N1OC1c1ccccc1] |
| 158 | 1G12 | 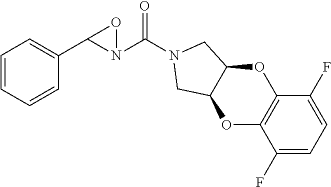 [SMILES: O=C(N1C[C@@H]2Oc3c(F)ccc(F)c3O[C@@H]2C1)N1OC1c1ccccc1] |
| 159 | 1H1 | 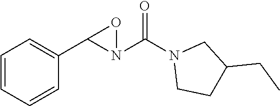 [SMILES: CCC1CCN(C(=O)N2OC2c2ccccc2)C1] |
| 160 | 1H2 | 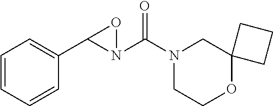 [SMILES: O=C(N1CCOC2(CCC2)C1)N1OC1c1ccccc1] |
| 161 | 1H3 | 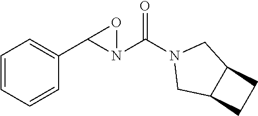 [SMILES: O=C(N1C[C@H]2CC[C@H]2C1)N1OC1c1ccccc1] |
| 162 | 1H5 | 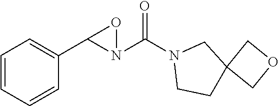 [SMILES: O=C(N1CCC2(COC2)C1)N1OC1c1ccccc1] |
| 163 | 1H6 | 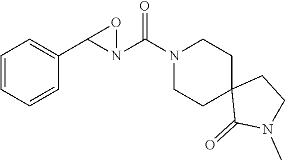 [SMILES: CN1CCC2(CCN(C(=O)N3OC3c3ccccc3)CC2)C1=O] |
| 164 | 1H7 | 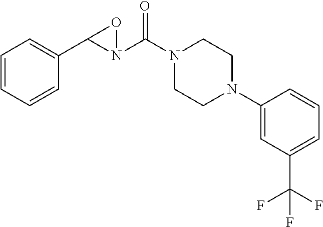 [SMILES: O=C(N1CCN(c2cccc(C(F)(F)F)c2)CC1)N1OC1c1ccccc1] |
| 165 | 1H8 | 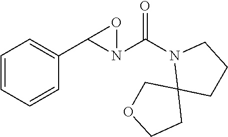 [SMILES: O=C(N1OC1c1ccccc1)N1CCCC12CCOC2] |
| 166 | 2A1 | 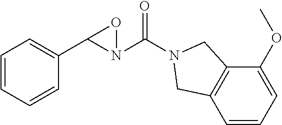 [SMILES: COc1cccc2c1CN(C(=O)N1OC1c1ccccc1)C2] |
| 167 | 2A2 | 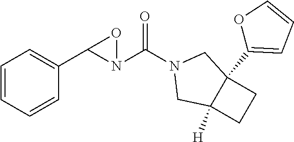 [SMILES: O=C(N1C[C@@H]2CC[C@]2(c2ccco2)C1)N1OC1c1ccccc1] |
| 168 | 2A4 | 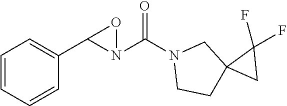 [SMILES: O=C(N1CCC2(C1)CC2(F)F)N1OC1c1ccccc1] |
| 169 | 2A5 | 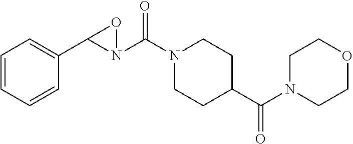 [SMILES: O=C(C1CCN(C(=O)N2OC2c2ccccc2)CC1)N1CCOCC1] |
| 170 | 2A6 | 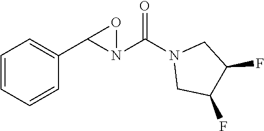 [SMILES: O=C(N1C[C@@H](F)[C@@H](F)C1)N1OC1c1ccccc1] |
| 171 | 2A9 | 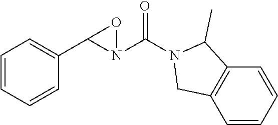 [SMILES: CC1c2ccccc2CN1C(=O)N1OC1c1ccccc1] |
| 172 | 2A10 | 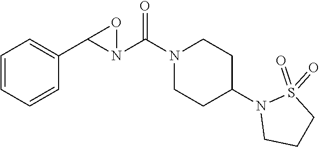 [SMILES: O=C(N1CCC(N2CCCS2(=O)=O)CC1)N1OC1c1ccccc1] |
| 173 | 2A12 | 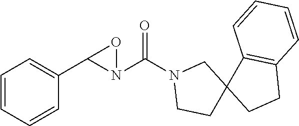 [SMILES: O=C(N1CCC2(CCc3ccccc32)C1)N1OC1c1ccccc1] |
| 174 | 2B1 | 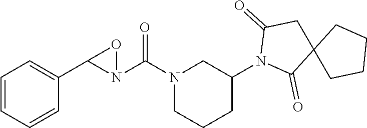 [SMILES: O=C(N1CCCC(N2C(=O)CC3(CCCC3)C2=O)C1)N1OC1c1ccccc1] |
| 175 | 2B2 | 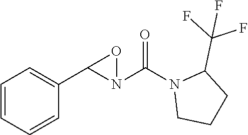 [SMILES: O=C(N1CCCC1C(F)(F)F)N1OC1c1ccccc1] |
| 176 | 2B3 | 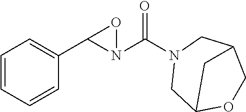 [SMILES: O=C(N1CC2COC(C2)C1)N1OC1c1ccccc1] |
| 177 | 2B5 | 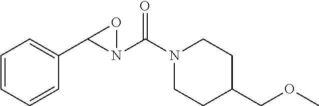 [SMILES: COCC1CCN(C(=O)N2OC2c2ccccc2)CC1] |
| 178 | 2B6 | 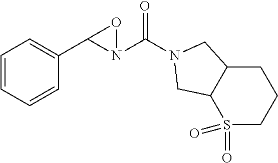 [SMILES: O=C(N1CC2CCCS(=O)(=O)C2C1)N1OC1c1ccccc1] |
| 179 | 2B9 | 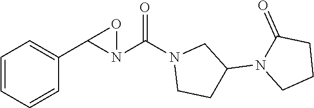 [SMILES: O=C1CCCN1C1CCN(C(=O)N2OC2c2ccccc2)C1] |
| 180 | 2B10 | 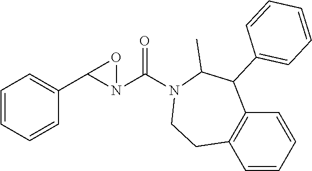 [SMILES: CC1C(c2ccccc2)c2ccccc2CCN1C(=O)N1OC1c1ccccc1] |
| 181 | 2B11 | 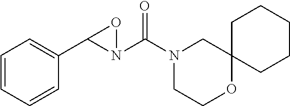 [SMILES: O=C(N1CCOC2(CCCCC2)C1)N1OC1c1ccccc1] |
| 182 | 2C2 | 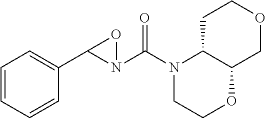 [SMILES: O=C(N1OC1c1ccccc1)N1CCO[C@H]2COCC[C@H]21] |
| 183 | 2C4 | 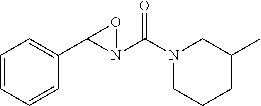 [SMILES: CC1CCCN(C(=O)N2OC2c2ccccc2)C1] |
| 184 | 2C6 | 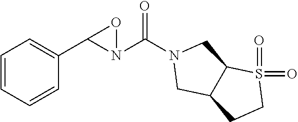 [SMILES: O=C(N1C[C@@H]2CCS(=O)(=O)[C@@H]2C1)N1OC1c1ccccc1] |
| 185 | 2C7 | 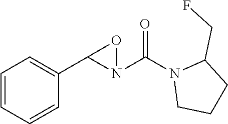 [SMILES: O=C(N1CCCC1CF)N1OC1c1ccccc1] |
| 186 | 2C9 | 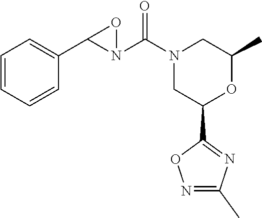 [SMILES: Cc1noc([C@H]2CN(C(=O)N3OC3c3ccccc3)C[C@@H](C)O2)n1] |
| 187 | 2C11 | 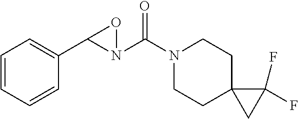 [SMILES: O=C(N1CCC2(CC1)CC2(F)F)N1OC1c1ccccc1] |
| 188 | 2C12 | 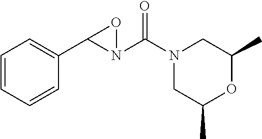 [SMILES: C[C@@H]1CN(C(=O)N2OC2c2ccccc2)C[C@H](C)O1] |
| 189 | 2D1 | 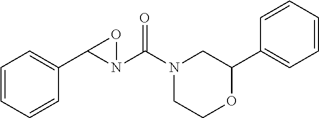 [SMILES: O=C(N1CCOC(c2ccccc2)C1)N1OC1c1ccccc1] |
| 190 | 2D2 | 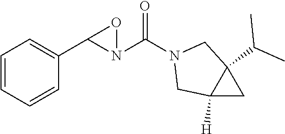 [SMILES: CC(C)[C@]12C[C@H]1CN(C(=O)N1OC1c1ccccc1)C2] |
| 191 | 2D4 | 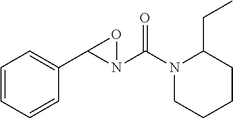 [SMILES: CCC1CCCCN1C(=O)N1OC1c1ccccc1] |
| 192 | 2D5 | 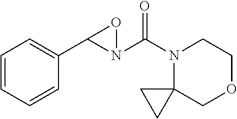 [SMILES: O=C(N1OC1c1ccccc1)N1CCOCC12CC2] |
| 193 | 2D6 | 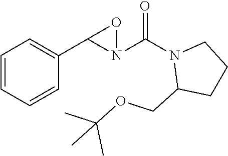 [SMILES: CC(C)(C)OCC1CCCN1C(=O)N1OC1c1ccccc1] |
| 194 | 2E1 | 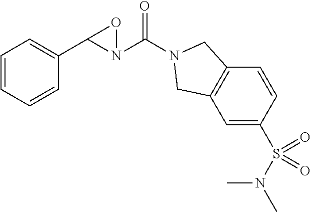 [SMILES: CN(C)S(=O)(=O)c1ccc2c(c1)CN(C(=O)N1OC1c1ccccc1)C2] |
| 195 | 2E3 | 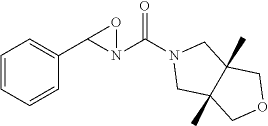 [SMILES: C[C@@]12COC[C@]1(C)CN(C(=O)N1OC1c1ccccc1)C2] |
| 196 | 2E5 | 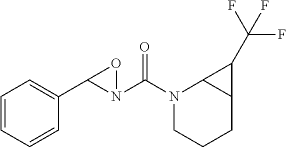 [SMILES: O=C(N1CCCC2C1C2C(F)(F)F)N1OC1c1ccccc1] |
| 197 | 2E8 | 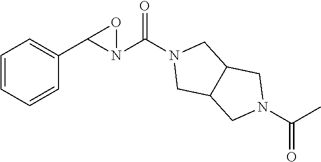 [SMILES: CC(=O)N1CC2CN(C(=O)N3OC3c3ccccc3)CC2C1] |
| 198 | 2E10 | 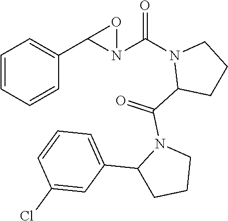 [SMILES: O=C(C1CCCN1C(=O)N1OC1c1ccccc1)N1CCCC1c1cccc(Cl)c1] |
| 199 | 2E11 | 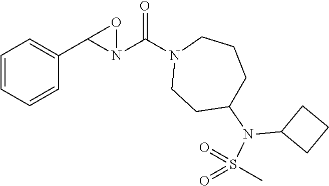 [SMILES: CS(=O)(=O)N(C1CCC1)C1CCCN(C(=O)N2OC2c2ccccc2)CC1] |
| 200 | 2E12 | 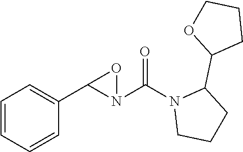 [SMILES: O=C(N1CCCC1C1CCCO1)N1OC1c1ccccc1] |
| 201 | 2F1 | 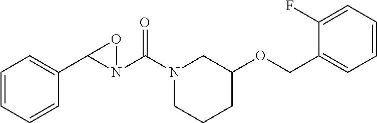 [SMILES: O=C(N1CCCC(OCc2ccccc2F)C1)N1OC1c1ccccc1] |
| 202 | 2F2 | 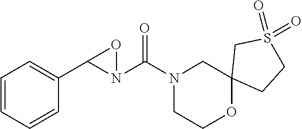 [SMILES: O=C(N1CCOC2(CCS(=O)(=O)C2)C1)N1OC1c1ccccc1] |
| 203 | 2F3 | 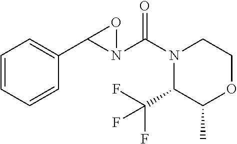 [SMILES: C[C@H]1OCCN(C(=O)N2OC2c2ccccc2)[C@H]1C(F)(F)F] |
| 204 | 2F4 | 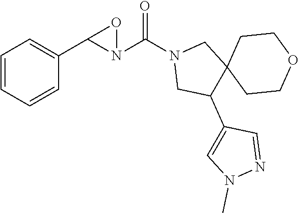 [SMILES: Cn1cc(C2CN(C(=O)N3OC3c3ccccc3)CC23CCOCC3)cn1] |
| 205 | 2F6 | 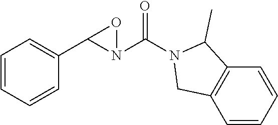 [SMILES: CC1c2ccccc2CN1C(=O)N1OC1c1ccccc1] |
| 206 | 2F8 | 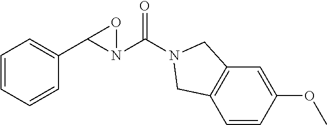 [SMILES: COc1ccc2c(c1)CN(C(=O)N1OC1c1ccccc1)C2] |
| 207 | 2F9 | 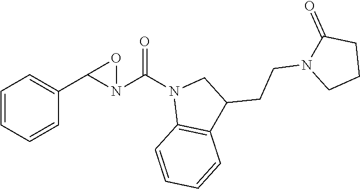 [SMILES: O=C1CCCN1CCC1CN(C(=O)N2OC2c2ccccc2)c2ccccc21] |
| 208 | 2F10 | 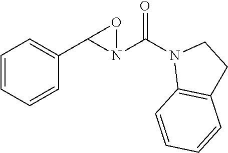 [SMILES: O=C(N1CCc2ccccc21)N1OC1c1ccccc1] |
| 209 | 2F12 | 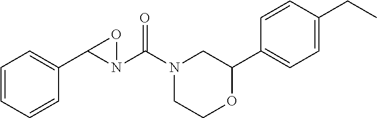 [SMILES: CCc1ccc(C2CN(C(=O)N3OC3c3ccccc3)CCO2)cc1] |
| 210 | 2G3 | 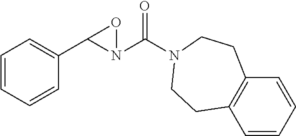 [SMILES: O=C(N1CCc2ccccc2CC1)N1OC1c1ccccc1] |
| 211 | 2G4 | 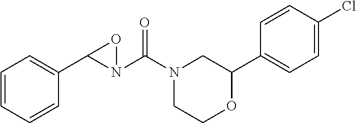 [SMILES: O=C(N1CCOC(c2ccc(Cl)cc2)C1)N1OC1c1ccccc1] |
| 212 | 2G6 | 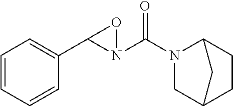 [SMILES: O=C(N1CC2CCC1C2)N1OC1c1ccccc1] |
| 213 | 2G7 | 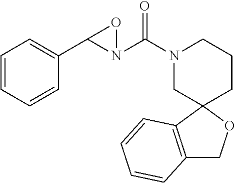 [SMILES: O=C(N1CCCC2(C1)OCc1ccccc12)N1OC1c1ccccc1] |
| 214 | 2G8 | 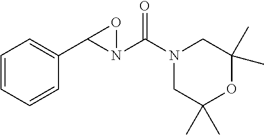 [SMILES: CC1(C)CN(C(=O)N2OC2c2ccccc2)CC(C)(C)O1] |
| 215 | 2G10 | 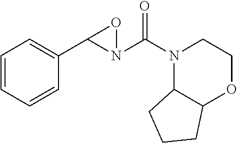 [SMILES: O=C(N1CCOC2CCCC21)N1OC1c1ccccc1] |
| 216 | 2G11 | 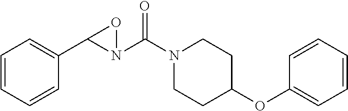 [SMILES: O=C(N1CCC(Oc2ccccc2)CC1)N1OC1c1ccccc1] |
| 217 | 2G12 | 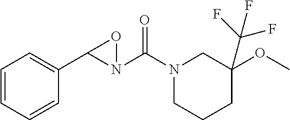 [SMILES: COC1(C(F)(F)F)CCCN(C(=O)N2OC2c2ccccc2)C1] |
| 218 | 2H1 | 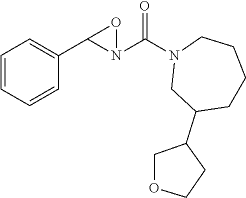 [SMILES: O=C(N1CCCCC(C2CCOC2)C1)N1OC1c1ccccc1] |
| 219 | 2H2 | 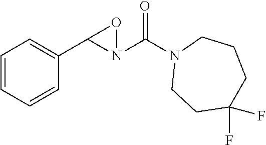 [SMILES: O=C(N1CCCC(F)(F)CC1)N1OC1c1ccccc1] |

In an embodiment, the compound is a compound provided in Table 1, or a pharmaceutically acceptable salt, hydrate, solvate, prodrug, stereoisomer, or tautomer thereof. In an embodiment, the compound is selected from one of Compound 100-110 in Table 1, or a pharmaceutically acceptable salt, hydrate, solvate, prodrug, stereoisomer, or tautomer thereof. In an embodiment, the compound is selected from one of Compound 110-120 in Table 1, or a pharmaceutically acceptable salt, hydrate, solvate, prodrug, stereoisomer, or tautomer thereof. In an embodiment, the compound is selected from one of Compound 120-130 in Table 1, or a pharmaceutically acceptable salt, hydrate, solvate, prodrug, stereoisomer, or tautomer thereof. In an embodiment, the compound is selected from one of Compound 130-140 in Table 1, or a pharmaceutically acceptable salt, hydrate, solvate, prodrug, stereoisomer, or tautomer thereof. In an embodiment, the compound is selected from one of Compound 140-150 in Table 1, or a pharmaceutically acceptable salt, hydrate, solvate, prodrug, stereoisomer, or tautomer thereof. In an embodiment, the compound is selected from one of Compound 150-160 in Table 1, or a pharmaceutically acceptable salt, hydrate, solvate, prodrug, stereoisomer, or tautomer thereof. In an embodiment, the compound is selected from one of Compound 160-170 in Table 1, or a pharmaceutically acceptable salt, hydrate, solvate, prodrug, stereoisomer, or tautomer thereof. In an embodiment, the compound is selected from one of Compound 170-180 in Table 1, or a pharmaceutically acceptable salt, hydrate, solvate, prodrug, stereoisomer, or tautomer thereof. In an embodiment, the compound is selected from one of Compound 180-190 in Table 1, or a pharmaceutically acceptable salt, hydrate, solvate, prodrug, stereoisomer, or tautomer thereof. In an embodiment, the compound is selected from one of Compound 190-200 in Table 1, or a pharmaceutically acceptable salt, hydrate, solvate, prodrug, stereoisomer, or tautomer thereof. In an embodiment, the compound is selected from one of Compound 200-210 in Table 1, or a pharmaceutically acceptable salt, hydrate, solvate, prodrug, stereoisomer, or tautomer thereof. In an embodiment, the compound is selected from one of Compound 210-219 in Table 1, or a pharmaceutically acceptable salt, hydrate, solvate, prodrug, stereoisomer, or tautomer thereof. In an embodiment, the compound is Compound 148 in Table 1, or a pharmaceutically acceptable salt, hydrate, solvate, prodrug, stereoisomer, or tautomer thereof. In an embodiment, the compound is Compound 138 in Table 1, or a pharmaceutically acceptable salt, hydrate, solvate, prodrug, stereoisomer, or tautomer thereof.

In another aspect, the present disclosure features a compound (e.g., an N-transfer oxidant compound) of Formula (II):

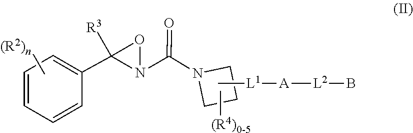

or a pharmaceutically acceptable salt, hydrate, solvate, prodrug, stereoisomer, or tautomer thereof, wherein R² is C₁-C₆ alkyl, C₂-C₆ alkenyl, C₂-C₆ alkynyl, C₁-C₆ heteroalkyl, C₁-C₆ haloalkyl, halo, cyano, or —OR^{A}; R³ is hydrogen, C₁-C₆ alkyl or halo; each of L¹ and L² is independently absent, C₁-C₆ alkylene, or C₁-C₆ heteroalkylene; A is absent, cycloalkyl, heterocyclyl, aryl, or heteroaryl, each of which is optionally substituted with one or more R⁵; B is absent, cycloalkyl, heterocyclyl, aryl, or heteroaryl, each of which is optionally substituted with one or more R⁵; each of R⁴ and R⁵ is independently C₁-C₆ alkyl, C₂-C₆ alkenyl, C₂-C₆ alkynyl, halo, cyano, —OR^{A}, or wherein two of R⁴ may come together to form a ring bound to the azetidinyl ring, or wherein or two of R⁵ may come together to form a ring with A or B respectively; R^{A} is hydrogen, C₁-C₆ alkyl, C₂-C₆ alkenyl, C₂-C₆ alkynyl, C₁-C₆ heteroalkyl, C₁-C₆ haloalkyl, cycloalkyl, or heterocyclyl; and n is 0, 1, 2, 3, 4, or 5; provided that if both L¹ and A are absent, then L² and B are absent.

In some embodiments, R² is hydrogen. In some embodiments, R² is halo. In some embodiments, R² is C₁-C₆ alkyl. In some embodiments, R³ is hydrogen. In some embodiments, R³ is halo. In some embodiments, R³ is C₁-C₆ alkyl.

In some embodiments, one of L¹ and L² is independently absent. In some embodiments, one of L¹ and L² is independently C₁-C₆ alkylene. In some embodiments, one of L¹ and L² is independently C₁-C₆ heteroalkylene. In some embodiments, L¹ is absent. In some embodiments, L² is absent. In some embodiments, L¹ is C₁-C₆ alkylene. In some embodiments, L² is C₁-C₆ alkylene. In some embodiments, L¹ is C₁-C₆ heteroalkylene. In some embodiments, L² is C₁-C₆ heteroalkylene. In some embodiments, one of L¹ and L² is independently C₁-C₆ alkylene. In some embodiments, one of L¹ and L² is independently C₁-C₆ heteroalkylene. In some embodiments, each of L¹ and L² is independently absent. In some embodiments, each of L¹ and L² is independently C₁-C₆ alkylene. In some embodiments, each of L¹ and L² is independently C₁-C₆ heteroalkylene.

In some embodiments, A is absent. In some embodiments, A is aryl, optionally substituted with one or more R⁵. In some embodiments, A is heteroaryl, optionally substituted with one or more R⁵. In some embodiments, A is a nitrogen-containing heteroaryl, optionally substituted with one or more R⁵. In some embodiments, A is heterocyclyl, optionally substituted with one or more R⁵. In some embodiments, A is a nitrogen-containing heterocyclyl, optionally substituted with one or more R⁵. In some embodiments, A is an oxygen-containing heterocyclyl, optionally substituted with one or more R⁵.

In some embodiments, B is absent. In some embodiments, B is aryl, optionally substituted with one or more R⁵. In some embodiments, B is heteroaryl, optionally substituted with one or more R⁵. In some embodiments, B is a nitrogen-containing heteroaryl, optionally substituted with one or more R⁵. In some embodiments, B is heterocyclyl, optionally substituted with one or more R⁵. In some embodiments, B is a nitrogen-containing heterocyclyl, optionally substituted with one or more R⁵. In some embodiments, B is an oxygen-containing heterocyclyl, optionally substituted with one or more R⁵.

In some embodiments, the compound of Formula (II) is a compound provided in any one of Tables 1, 2, or 3. In some embodiments, the compound of Formula (II) is a compound provided in Table 1. In some embodiments, the compound of Formula (II) is a compound provided in Table 2. In some embodiments, the compound of Formula (II) is a compound provided in Table 3.

TABLE 2
Exemplary Compounds of Formula (II)

| Cmpd No. | NVS Ref No. | Structure |
|---|---|---|
| 220 | 1oxA2 | 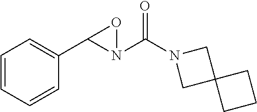 [SMILES: O=C(N1CC2(CCC2)C1)N1OC1c1ccccc1] |
| 221 | 1oxC7 | 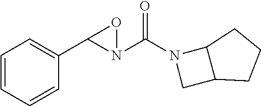 [SMILES: O=C(N1CC2CCCC21)N1OC1c1ccccc1] |
| 222 | 1oxD1 | 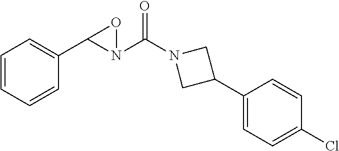 [SMILES: O=C(N1CC(c2ccc(Cl)cc2)C1)N1OC1c1ccccc1] |
| 223 | 1oxD3 | 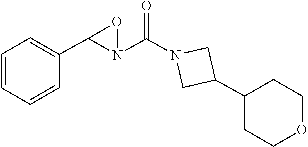 [SMILES: O=C(N1CC(C2CCOCC2)C1)N1OC1c1ccccc1] |
| 224 | 1oxD7 | 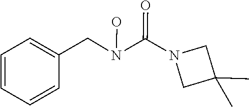 |
| 225 | 1oxD11 | 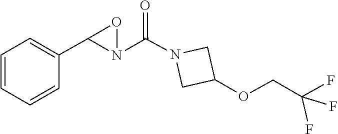 [SMILES: O=C(N1CC(OCC(F)(F)F)C1)N1OC1c1ccccc1] |
| 226 | loxD12 | 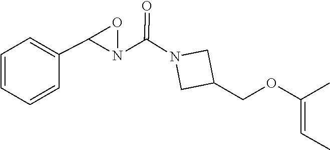 [SMILES: C/C=C(\C)OCC1CN(C(=O)N2OC2c2ccccc2)C1] |
| 227 | 1oxE3 | 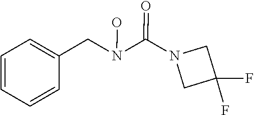 |
| 228 | 1oxE7 | 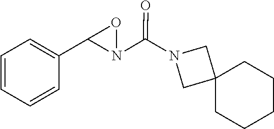 [SMILES: O=C(N1CC2(CCCCC2)C1)N1OC1c1ccccc1] |
| 229 | 1F3 | 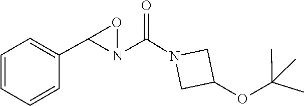 [SMILES: CC(C)(C)OC1CN(C(=O)N2OC2c2ccccc2)C1] |
| 230 | 1F5 | 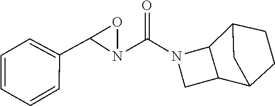 |
| 231 | 1H4 | 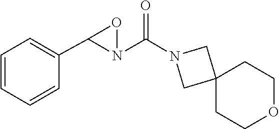 [SMILES: O=C(N1CC2(CCOCC2)C1)N1OC1c1ccccc1] |
| 232 | 2A3 | 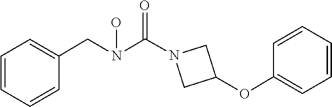 |
| 233 | 2A8 | 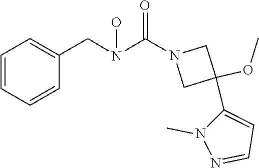 |
| 234 | 2B4 | 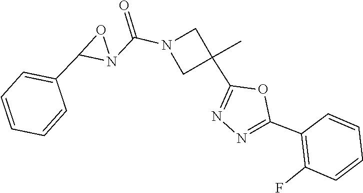 [SMILES: CC1(c2nnc(-c3ccccc3F)o2)CN(C(=O)N2OC2c2ccccc2)C1] |
| 235 | 2C5 | 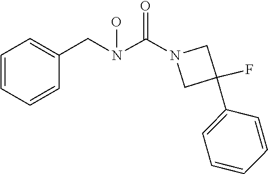 |
| 236 | 2C10 | 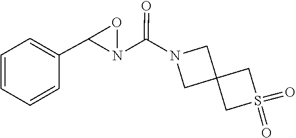 [SMILES: O=C(N1CC2(C1)CS(=O)(=O)C2)N1OC1c1ccccc1] |
| 237 | 2D10 | 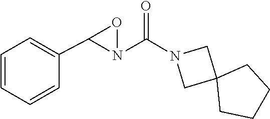 [SMILES: O=C(N1CC2(CCCC2)C1)N1OC1c1ccccc1] |
| 238 | 2D11 | 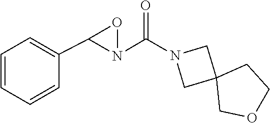 [SMILES: O=C(N1CC2(CCOC2)C1)N1OC1c1ccccc1] |
| 239 | 2E9 | 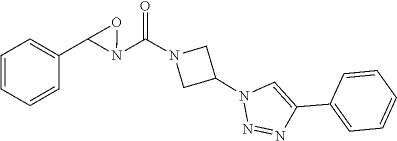 |
| 240 | 2G2 | 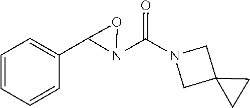 |
| 241 | 2G5 | 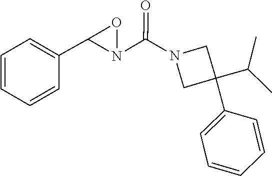 [SMILES: CC(C)C1(c2ccccc2)CN(C(=O)N2OC2c2ccccc2)C1] |
| 242 | 2G9 | 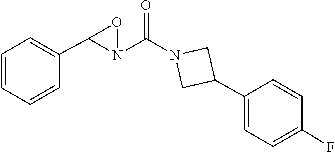 [SMILES: O=C(N1CC(c2ccc(F)cc2)C1)N1OC1c1ccccc1] |

In an embodiment, the compound is a compound provided in Table 2, or a pharmaceutically acceptable salt, hydrate, solvate, prodrug, stereoisomer, or tautomer thereof. In an embodiment, the compound is selected from one of Compound 220-230 in Table 2, or a pharmaceutically acceptable salt, hydrate, solvate, prodrug, stereoisomer, or tautomer thereof. In an embodiment, the compound is selected from one of Compound 230-242 in Table 2, or a pharmaceutically acceptable salt, hydrate, solvate, prodrug, stereoisomer, or tautomer thereof.

In another aspect, the present disclosure features a compound (e.g., an N-transfer oxidant compound) of Formula (III):

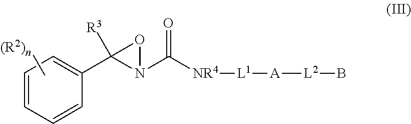

or a pharmaceutically acceptable salt, hydrate, solvate, prodrug, stereoisomer, or tautomer thereof, wherein R² is C₁-C₆ alkyl, C₂-C₆ alkenyl, C₂-C₆ alkynyl, C₁-C₆ heteroalkyl, C₁-C₆ haloalkyl, halo, cyano, or —OR^{A}; R³ is hydrogen, C₁-C₆ alkyl or halo; each of L¹ and L² is independently absent, C₁-C₆ alkylene, or C₁-C₆ heteroalkylene; A is absent, cycloalkyl, heterocyclyl, aryl, or heteroaryl, each of which is optionally substituted with one or more R⁵; B is absent, cycloalkyl, heterocyclyl, aryl, or heteroaryl, each of which is optionally substituted with one or more R⁵; R⁴ is independently hydrogen, C₁-C₆ alkyl, or cycloalkyl; each R⁵ is independently C₁-C₆ alkyl, C₂-C₆ alkenyl, C₂-C₆ alkynyl, halo, cyano, —OR^{A}, or wherein two of R⁵ may come together to form a ring with A or B respectively; R^{A} is hydrogen, C₁-C₆ alkyl, C₂-C₆ alkenyl, C₂-C₆ alkynyl, C₁-C₆ heteroalkyl, C₁-C₆ haloalkyl, cycloalkyl, or heterocyclyl; and n is 0, 1, 2, 3, 4, or 5; provided that if both L¹ and A are absent, then L² and B are absent.

In some embodiments, R² is hydrogen. In some embodiments, R² is halo. In some embodiments, R² is C₁-C₆ alkyl. In some embodiments, R³ is hydrogen. In some embodiments, R³ is halo. In some embodiments, R³ is C₁-C₆ alkyl.

In some embodiments, one of L¹ and L² is independently absent. In some embodiments, one of L¹ and L² is independently C₁-C₆ alkylene. In some embodiments, one of L¹ and L² is independently C₁-C₆ heteroalkylene. In some embodiments, L¹ is absent. In some embodiments, L² is absent. In some embodiments, L¹ is C₁-C₆ alkylene. In some embodiments, L² is C₁-C₆ alkylene. In some embodiments, L¹ is C₁-C₆ heteroalkylene. In some embodiments, L² is C₁-C₆ heteroalkylene. In some embodiments, one of L¹ and L² is independently C₁-C₆ alkylene. In some embodiments, one of L¹ and L² is independently C₁-C₆ heteroalkylene. In some embodiments, each of L¹ and L² is independently absent. In some embodiments, each of L¹ and L² is independently C₁-C₆ alkylene. In some embodiments, each of L¹ and L² is independently C₁-C₆ heteroalkylene.

In some embodiments, A is absent. In some embodiments, A is aryl, optionally substituted with one or more R⁵. In some embodiments, A is heteroaryl, optionally substituted with one or more R⁵. In some embodiments, A is a nitrogen-containing heteroaryl, optionally substituted with one or more R⁵. In some embodiments, A is heterocyclyl, optionally substituted with one or more R⁵. In some embodiments, A is a nitrogen-containing heterocyclyl, optionally substituted with one or more R⁵. In some embodiments, A is an oxygen-containing heterocyclyl, optionally substituted with one or more R⁵.

In some embodiments, B is absent. In some embodiments, B is aryl, optionally substituted with one or more R⁵. In some embodiments, B is heteroaryl, optionally substituted with one or more R⁵. In some embodiments, B is a nitrogen-containing heteroaryl, optionally substituted with one or more R⁵. In some embodiments, B is heterocyclyl, optionally substituted with one or more R⁵. In some embodiments, B is a nitrogen-containing heterocyclyl, optionally substituted with one or more R⁵. In some embodiments, B is an oxygen-containing heterocyclyl, optionally substituted with one or more R⁵.

In some embodiments, the compound of Formula (III) is a compound provided in any one of Tables 1, 2, or 3. In some embodiments, the compound of Formula (III) is a compound provided in Table 1. In some embodiments, the compound of Formula (III) is a compound provided in Table 2. In some embodiments, the compound of Formula (III) is a compound provided in Table 3.

TABLE 3
Exemplary compounds of Formula (III)

| Cmpd No. | NVS Ref No. | Structure |
|---|---|---|
| 250 | 1oxA6 | 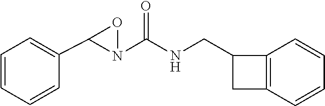 [SMILES: O=C(NCC1Cc2ccccc21)N1OC1c1ccccc1] |
| 251 | 1oxB1 | 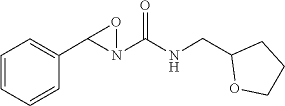 |
| 252 | 1oxB2 | 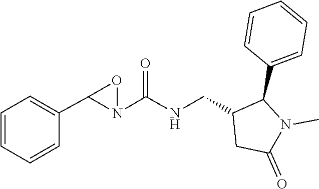 |
| 253 | 1oxB5 | 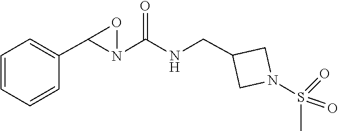 [SMILES: CS(=O)(=O)N1CC(CNC(=O)N2OC2c2ccccc2)C1] |
| 254 | 1oxB8 | 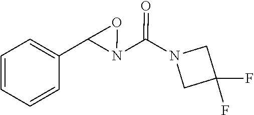 |
| 255 | 1oxB10 | 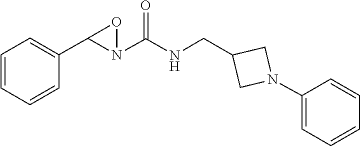 [SMILES: O=C(NCC1CN(c2ccccc2)C1)N1OC1c1ccccc1] |
| 256 | 1oxB12 | 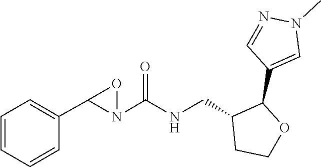 [SMILES: Cn1cc([C@H]2OCC[C@@H]2CNC(=O)N2OC2c2ccccc2)cn1] |
| 257 | 1oxC6 | 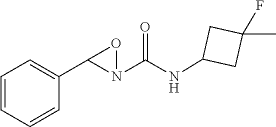 [SMILES: CC1(F)CC(NC(=O)N2OC2c2ccccc2)C1] |
| 258 | 1oxC10 | 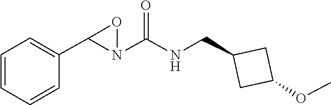 [SMILES: CO[C@H]1C[C@H](CNC(=O)N2OC2c2ccccc2)C1] |
| 259 | 1oxE9 | 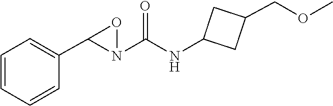 [SMILES: COCC1CC(NC(=O)N2OC2c2ccccc2)C1] |
| 260 | 1F4 | 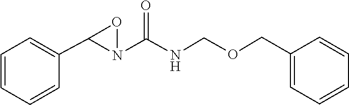 [SMILES: O=C(NCOCc1ccccc1)N1OC1c1ccccc1] |
| 261 | 1G8 | 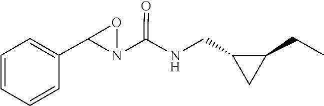 [SMILES: CC[C@H]1C[C@@H]1CNC(=O)N1OC1c1ccccc1] |
| 262 | 1G9 | 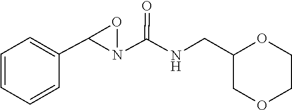 [SMILES: O=C(NCC1COCCO1)N1OC1c1ccccc1] |
| 263 | 1G10 | 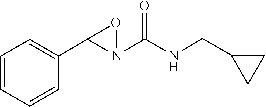 |
| 264 | 2A7 | 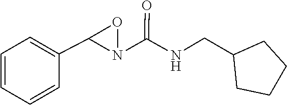 [SMILES: O=C(NCC1CCCC1)N1OC1c1ccccc1] |
| 265 | 2A11 | 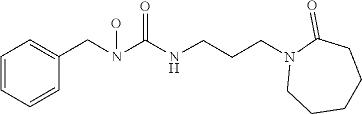 |
| 266 | 2B7 | 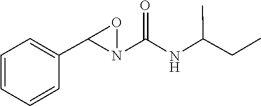 [SMILES: CCC(C)NC(=O)N1OC1c1ccccc1] |
| 267 | 2B8 | 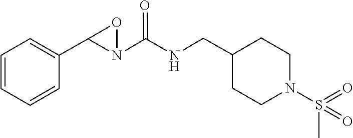 [SMILES: CS(=O)(=O)N1CCC(CNC(=O)N2OC2c2ccccc2)CC1] |
| 268 | 2B12 | 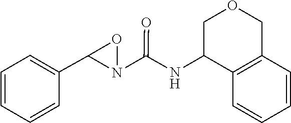 [SMILES: O=C(NC1COCc2ccccc21)N1OC1c1ccccc1] |
| 269 | 2C1 | 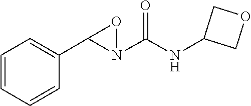 |
| 270 | 2C3 | 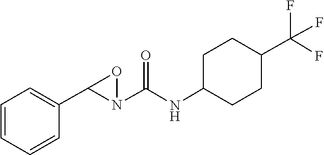 [SMILES: O=C(NC1CCC(C(F)(F)F)CC1)N1OC1c1ccccc1] |
| 271 | 2C8 | 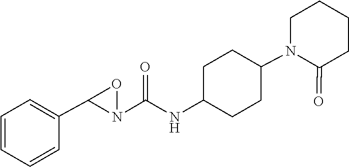 [SMILES: O=C1CCCCN1C1CCC(NC(=O)N2OC2c2ccccc2)CC1] |
| 272 | 2D3 | 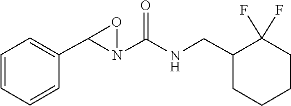 [SMILES: O=C(NCC1CCCCC1(F)F)N1OC1c1ccccc1] |
| 273 | 2D7 | 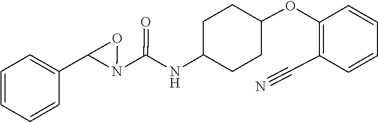 |
| 274 | 2D8 | 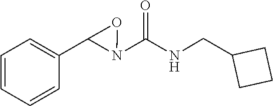 |
| 275 | 2D9 | 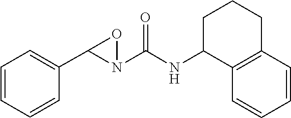 [SMILES: O=C(NC1CCCc2ccccc21)N1OC1c1ccccc1] |
| 276 | 2D12 | 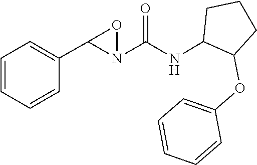 [SMILES: O=C(NC1CCCC1Oc1ccccc1)N1OC1c1ccccc1] |
| 277 | 2E2 | 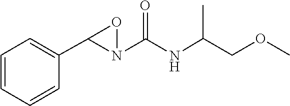 [SMILES: COCC(C)NC(=O)N1OC1c1ccccc1] |
| 278 | 2E4 | 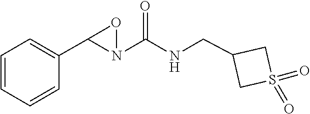 [SMILES: O=C(NCC1CS(=O)(=O)C1)N1OC1c1ccccc1] |
| 279 | 2E6 | 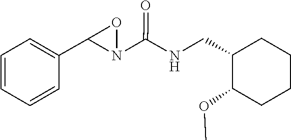 [SMILES: CO[C@H]1CCCC[C@H]1CNC(=O)N1OC1c1ccccc1] |
| 280 | 2E7 | 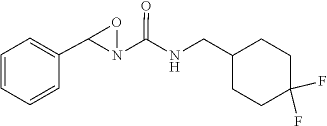 [SMILES: O=C(NCC1CCC(F)(F)CC1)N1OC1c1ccccc1] |
| 281 | 2F5 | 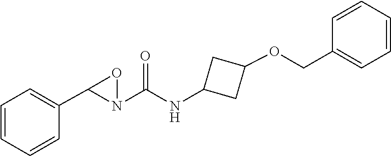 [SMILES: O=C(NC1CC(OCc2ccccc2)C1)N1OC1c1ccccc1] |
| 282 | 2F7 | 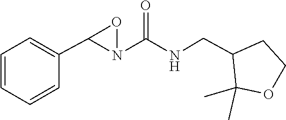 [SMILES: CC1(C)OCCC1CNC(=O)N1OC1c1ccccc1] |
| 283 | 2F11 | 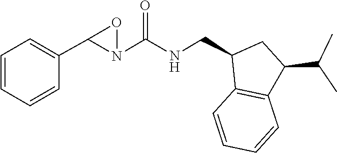 [SMILES: CC(C)[C@@H]1C[C@H](CNC(=O)N2OC2c2ccccc2)c2ccccc21] |
| 284 | 2G1 | 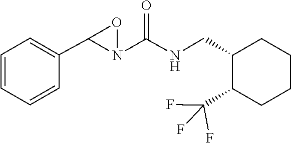 [SMILES: O=C(NC[C@@H]1CCCC[C@@H]1C(F)(F)F)N1OC1c1ccccc1] |
| 285 | 2H3 | 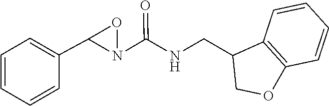 [SMILES: O=C(NCC1COc2ccccc21)N1OC1c1ccccc1] |

In an embodiment, the compound is a compound provided in Table 3, or a pharmaceutically acceptable salt, hydrate, solvate, prodrug, stereoisomer, or tautomer thereof. In an embodiment, the compound is selected from one of Compound 243-250 in Table 3, or a pharmaceutically acceptable salt, hydrate, solvate, prodrug, stereoisomer, or tautomer thereof. In an embodiment, the compound is selected from one of Compound 250-260 in Table 3, or a pharmaceutically acceptable salt, hydrate, solvate, prodrug, stereoisomer, or tautomer thereof. In an embodiment, the compound is selected from one of Compound 260-270 in Table 3, or a pharmaceutically acceptable salt, hydrate, solvate, prodrug, stereoisomer, or tautomer thereof. In an embodiment, the compound is selected from one of Compound 270-280 in Table 3, or a pharmaceutically acceptable salt, hydrate, solvate, prodrug, stereoisomer, or tautomer thereof. In an embodiment, the compound is selected from one of Compound 280-285 in Table 3, or a pharmaceutically acceptable salt, hydrate, solvate, prodrug, stereoisomer, or tautomer thereof.

### Pharmaceutically Acceptable Salts

Pharmaceutically acceptable salts of the compounds described herein are also contemplated for the uses described herein. As used herein, the terms “salt” or “salts” refer to an acid addition or base addition salt of a compound described herein. “Salts” include in particular “pharmaceutical acceptable salts.” The term “pharmaceutically acceptable salts” refers to salts that retain the biological effectiveness and properties of the compounds disclosed herein and, which typically are not biologically or otherwise undesirable. In many cases, the compounds disclosed herein are capable of forming acid and/or base salts by virtue of the presence of amino and/or carboxyl groups or groups similar thereto.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium, and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine, and tromethamine.

In some embodiments, the bifunctional compound of Formula (I) is provided as an acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate, or xinafoate salt form.

### Pharmaceutical Compositions

Another embodiment is a pharmaceutical composition comprising one or more compounds described herein or a pharmaceutically acceptable salt, hydrate, solvate, prodrug, stereoisomer, or tautomer thereof, and one or more pharmaceutically acceptable carrier(s). The term “pharmaceutically acceptable carrier” refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions of the disclosure are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tween®, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax, and polyethylene glycols.

The pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water.

The pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. The amount of the compounds of the present disclosure that may be combined with the carrier materials to produce a composition in a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

### Isotopically Labelled Compounds

A compound described herein or a pharmaceutically acceptable salt, hydrate, solvate, prodrug, stereoisomer, or tautomer thereof, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds described herein include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F, ³¹P, ³²P, ³⁵, ³⁶Cl, ¹²³I, ¹²⁴I, ¹²⁵I, respectively. The disclosure includes various isotopically labeled compounds as defined herein, for example, those into which radioactive isotopes, such as ³H and ⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds described herein or a pharmaceutically acceptable salt, hydrate, solvate, prodrug, stereoisomer, or tautomer thereof, can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example, increased in vivo half-life or reduced dosage requirements or an improvement in therapeutic index. It is understood that deuterium in this context is regarded as a substituent of a compound described herein or a pharmaceutically acceptable salt, hydrate, solvate, prodrug, stereoisomer, or tautomer thereof. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound described herein is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Methods of Use

The disclosure provides compounds, compositions and related methods for targeting a methionine residue in a peptide or protein, e.g., a functional allosteric methionine residue in a target protein. In an aspect, the disclosure provides an activity-based protein profiling (ABPP) method comprising activity-based profiling of a target protein using Redox-Activated Chemical Tagging (ReACT) for bioconjugation by targeting a methionine within a protein through the use of oxaziridine reagents that promote selective nitrene fragment transfer reactivity that is isoelectronic to native methionine oxidation by oxygen atom transfer.

In an embodiment, the target protein is an enzyme. In an embodiment, the target protein is an enzyme, and the methionine residue is located on the surface (e.g., solvent-exposed region) of the protein. In an embodiment, the target protein is an enzyme, and the methionine residue is an allosteric methionine residue. In an embodiment, the target protein is an enzyme, and the target protein is a catalytic methionine residue. In an embodiment, the labeling of target protein at a methionine residue with a compound described herein (e.g., a compound of Formula (I), (I-a), (II), or (III)) does not alter the activity of the protein, e.g., relative to the activity of the target protein in the absence of a compound described herein. In an embodiment, the labeling of target enzyme at a methionine residue with a compound described herein (e.g., a compound of Formula (I), (I-a), (II), or (III)) does not alter the activity of the enzyme, e.g., relative to the activity of the target protein in the absence of a compound described herein. In an embodiment, the labeling of target protein at a methionine residue with a compound described herein (e.g., a compound of Formula (I), (I-a), (II), or (III)) does not alter the interaction of the target protein with another entity, e.g., another protein or small molecule, e.g., relative to the activity of the target protein in the absence of a compound described herein. In an embodiment, the labeling of target enzyme at a methionine residue with a compound described herein (e.g., a compound of Formula (I), (I-a), (II), or (III)) does not alter the interaction of the target enzyme with another entity, e.g., another protein or small molecule, e.g., relative to the activity of the target protein in the absence of a compound described herein.

In an embodiment, the target protein comprises a single methionine residue. In an embodiment, the target protein comprises a plurality of methionine residues (e.g., about 2, 3, 4, 5, 6, 7, 8, 9, 10, 11, 12, 13, 14, 15, 16, 17, 18, 19, 20, or more methionine residues). In an embodiment, the target protein comprises 2 methionine residues. In an embodiment, the target protein comprises 3 methionine residues. In an embodiment, the target protein comprises 4 methionine residues. In an embodiment, the target protein comprises 5 methionine residues. In an embodiment, the target protein comprises 6 methionine residues. In an embodiment, the target protein comprises 7 methionine residues. In an embodiment, the target protein comprises 8 methionine residues. In an embodiment, the target protein comprises 9 methionine residues. In an embodiment, the target protein comprises 10 methionine residues. In an embodiment, the target protein comprises more than 10 methionine residues. In an embodiment, the target protein comprises more than 15 methionine residues.

In an embodiment, the target protein is a kinase. In an embodiment, the target protein is a hydrolase. In an embodiment, the target protein is a transferase. In an embodiment, the target protein is a phosphatase. In an embodiment, the target protein is a ligase. In an embodiment, the target protein is an oxidoreductase. In an embodiment, the target protein is an isomerase.

In an embodiment, the target protein is a cyclin-dependent kinase. In an embodiment, the target protein is selected from CDK1, CDK2, CDK3, and CDK4. In embodiments, the target protein is CDK1. In embodiments, the target protein is CDK2. In embodiments, the target protein is CDK3. In embodiments, the target protein is CDK4.

In another aspect, the present disclosure features kits for targeting a methionine residue in a peptide or protein, e.g., a functional allosteric methionine residue in a target protein. In an embodiment, the kit described herein contains a compound described herein (e.g., a compound of Formula (I), (II), or (III)).

In another aspect, the present disclosure features a methionine-directed ABPP platform for identifying and developing covalent ligands for new functional methionine sites. One value of this approach using CDK4 as a representative high-value target to showcase the application of ReACT probes to reveal fundamental new chemical function on proteins and accelerate drug discovery efforts by expanding covalent ligand development beyond the more common cysteine and lysine protein nucleophiles.

As described herein, methionine-directed ReACT probes with broad reactivity were applied to CDK4 to identify novel hyperreactive, ligandable methionine sites on CDK4.

The synthesis of oxaziridine probes were optimized and these methods were used to design and synthesize a focused covalent ligand library of ca. 180 oxaziridine fragments bearing chemically diverse functional groups, including spirocycles, halogens, azoles, ethers, and amides. Synthesis of the fragment library was guided by computational design to ensure efficient N-transfer rates and sulfimine stability of the subsequent products with methionine.

This ReACT ABPP platform was established to be useful for fragment-based screening efforts against the representative oncoprotein CDK4. Chemoproteomic experiments revealed that Compound 148 was a covalent modifier of CDK4 that selectively labeled its allosteric M169 site with isoform specificity over CDK1 and CDK6. Biochemical and cell-based assays showed that Compound 148 can inhibit CDK4 activity on purified protein and in cells and decrease cell viability in a dose-dependent manner, with detection of target engagement in cells enabled by the synthesis of Compound 300, bearing an alkyne handle for detection and enrichment.

Further biochemical studies uncovered a novel redox regulatory mechanism for kinase inactivation through reciprocal oxidation/phosphorylation crosstalk between proximal M169 and T172 residues in CDK4, where M169 oxidation hinders phosphorylation at the protein-activating T172 site. Use of a phospho-specific pT172-CDK4 antibody in 1D- and 2D-western blot analyses established that treatment with the M169-modifying Compound 148 covalent ligand diminished T172 phosphorylation and CDK4 activity. The resulting loss of CDK4 function prevented downstream Rb phosphorylation at S780 and S807/811, leading to cell cycle arrest by failure at the S-phase checkpoint.

These findings support a role for M169 as a redox sensor site at the S-phase checkpoint, preventing cell division under highly oxidative conditions by sterically preventing phosphorylation at T172. This newly discovered redox vulnerability in CDK4 provides an alternative modality for therapeutic intervention.

In another aspect, the present disclosure provides a method of chemoselective conjugation comprising reacting the N-transfer oxidant compound disclosed herein with a thioether substrate in an aqueous (preferably >90% or 95% water), biocompatible environment to form a conjugation product comprising a resultant sulfimide.

The biocompatible environment is generally non-denaturing and generally compatible with the preservation of protein structure and function; and in particular, as applied to the subject proteins of the reaction. The conditions are distinct from reactions in generally denaturing organic solvents, with simple thioethers, where many different chemical products are formed.

In embodiments, the thioether substrate is a methionine substrate and the method provides a residue-specific bioconjugation strategy for methionine-based substrate functionalization.

In embodiments, the thioether substrate is a methionine substrate of a peptide, a polypeptide, or a protein.

In embodiments, the thioether substrate is a methionine substrate of a peptide, a polypeptide, or a protein and the method results in site- and residue-specific modification of the protein, with applications in synthesis and characterization of antibody-drug conjugates and related biologic therapeutics and imaging agents, chemoproteomics and inhibitor design, as well as modifications to study and improve upon protein function, including solubility, stability, and metabolism and pharmacokinetics.

In embodiments, the thioether substrate is a methionine substrate of a peptide, a polypeptide, or a protein is an antibody, adeno-associated virus (AAV) capsid protein; the antibody is selected from a single-chain variable fragment antibody, a designed ankyrin repeat proteins (DARPin), and a single variable domain on a heavy chain (VHH) antibody.

The disclosure encompasses all combinations of the particular embodiments recited herein, as if each combination had been laboriously recited.

### Examples

In order that the invention described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein. Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC).

Reactions using moisture- or air-sensitive reagents were carried out in flame-dried glassware under an inert atmosphere of N₂. All non-aqueous reactions were performed under an inert atmosphere of dry nitrogen in flame dried glassware sealed with a rubber septum unless stated otherwise. Nitrogen was supplied through a glass manifold. Solvent was passed over activated alumina and stored over activated 3 Å molecular sieves before use when dry solvent was required. All other commercially purchased chemicals were used as received (without further purification). Reactions were stirred magnetically and monitored by thin layer chromatography (TLC).

TLC: Analytical thin layer chromatography (TLC) was performed using MERCK Silica Gel 60 F254 TLC glass plates and visualized by ultraviolet light (UV). SiliCycle 60 F254 silica gel pre-coated sheets (0.25 mm thick) were used for analytical thin layer chromatography and visualized by fluorescence quenching under UV light. Additionally, TLC plates were stained with aqueous potassium permanganate (KMnO4) [1.5 g KMnO4, 200 mL H2O, 10 g K2CO3, 1.25 mL 10% NaOH].

Concentration under reduced pressure was performed by rotator evaporation at 40° C. at the appropriate pressure.

Column Chromatography: Chromatographic purification was performed as flash chromatography on MERCK silica gel 60 Å(230×400 mesh) at 0.2-0.5 bar overpressure.

Purified compounds were dried further under high vacuum (0.01-0.1 mbar).

Yields refer to the purified compound.

¹H NMR: Nuclear Magnetic Resonance (NMR) spectra were recorded on BRUKER AV (600 MHz and 300 MHz), AVB (400 MHz), AVQ (400 MHz) and NEO (500 MHz) spectrometers. Measurements were carried out at ambient temperature. All chemical shifts (6) are reported in the standard notation of 6, parts per million (ppm) relative to the residual solvent peak signal as internal standard (chloroform at 7.26 and 77.00 ppm for ¹H NMR and ¹³C NMR spectroscopy, respectively). The data is reported as (s=singlet, d=doublet, t=triplet, q=quartet, p=quintet, m=multiplet or unresolved, br=broad signal, dd=doublet of doublets, coupling constant(s) in Hz, integration). ¹³C NMR spectra were recorded with broadband ¹H decoupling.

LC-MS: Low-resolution electrospray mass spectral analyses were carried out using LC-MS (Agilent Technology 6130, Quadrupole LC/MS and Advion Expression-L Compact Mass Spectrometer). High-resolution mass spectral analyses (ESI-MS) were carried out at the College of Chemistry Mass Spectrometry Facility at the University of California, Berkeley.

Compounds 301, 302, and 303 were synthesized using published procedures (see, e.g., Lin, S et al.;. Science 2017, 355 (6325), 597-602).

All aqueous solutions were prepared using Milli-Q water, and all in vitro experiments were carried out in PBS, pH 7.4, unless otherwise noted. All biological experiments were prepared using freshly prepared aliquots.

### General Synthetic Schemes

Compounds of the present disclosure may be prepared using a synthetic protocol illustrated in Scheme A below.

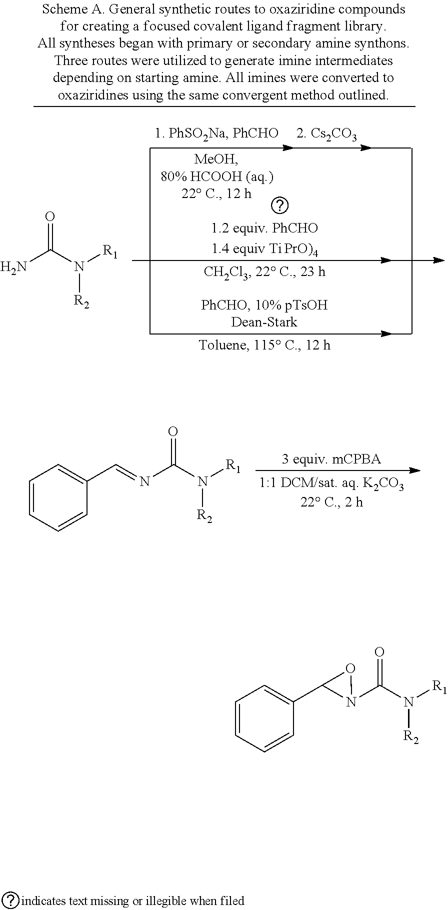

### Example 1. General Synthesis of Compounds of Formula (I)

### Method 1 for the Synthesis of Imine Intermediate A-1

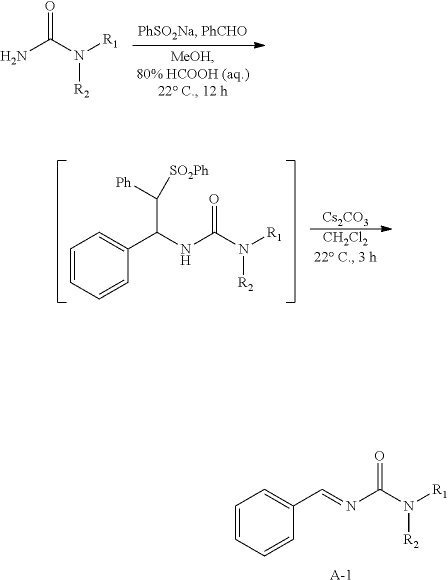

A solution of desired urea (10 mmol) and PhSO₂Na (2 eq) was dissolved in water (20 mL), with stirring. PhCHO (2 eq) in MeOH (8 mL) was then added thereto, followed by aqueous 80% HCOOH (0.8 mL). The reaction was stirred at room temperature overnight. If a precipitate was formed, it was filtered, washed with water and hexanes, and dried. If no precipitate was observed, HCl was added to the reaction mixture until reaching pH 3. In most exemplary cases, after overnight stirring, the precipitate was formed and collected after filtration, washing with water and hexanes, and dried. The imine collected was then used directly in the synthesis of the oxaziridine compounds.

### Method 2 for the Synthesis of Imine Intermediate A-1

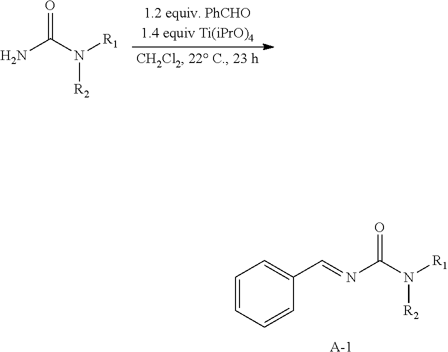

To a solution of corresponding urea (115 mmol) and benzaldehyde (127 mmol) in dry CH₂Cl₂ (20 mL) was added Ti(iPrO)₄ (140 mmol) dropwise at room temperature. The mixture was stirred at room temperature for 23 h. The solvent was removed in vacuo to afford crude imine which was used immediately towards the next step of forming the oxaziridine ligand. The reaction was monitored by 1H NMR via appearance of the imine proton signal intensity.

### Method 3 for the Synthesis of Imine Intermediate A-1

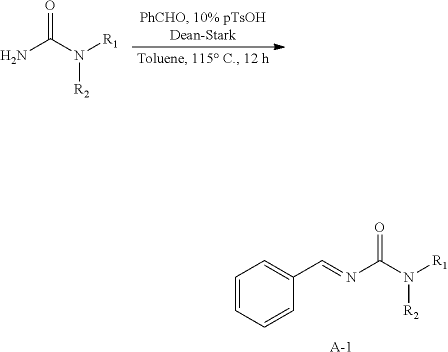

To a flame-dried 3-neck round bottom equipped with a Dean-Stark trap and reflux condenser, was added urea (10 mmol, 1 equiv.) and toluene (200 mL). To this mixture was added benzaldehyde (25 mmol, 2.5 equiv.) and pTsOH·H₂O (2 mmol, 0.2 equiv.), and the mixture was refluxed for 12 hours. The mixture was then concentrated under reduced pressure and was used in the next step of oxaziridine synthesis without further purification.

### Synthesis of Oxaziridine Compounds

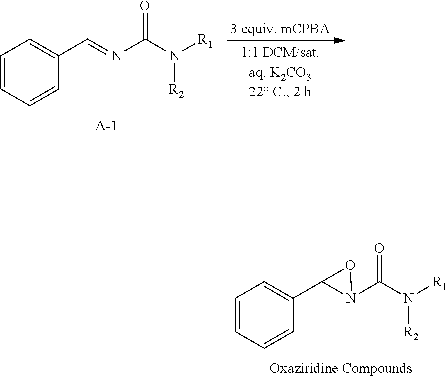

In a 25 mL round-bottom flask, mCPBA (75%, 538 mmol) was pre-stirred in 1:1 CH₂Cl₂/sat. aq. K₂CO₃ (140 mL) at room temperature for 10 minutes. A solution of imine was added rapidly dropwise, using CH₂Cl₂. If a partial dissolution of solids was observed after imine addition, sat. K₂CO₃ was added to reaction mixture immediately. After 2 h, the reaction was diluted with water (150 mL) and extracted with CH₂Cl₂ (3×100 mL). The organic layer was collected, washed with diluted aq. K₂CO₃ (3 times) and water (2 times), dried over Na₂SO₄, filtered, and concentrated in vacuo. The residue was purified by column chromatography.

Compound 148, an exemplary compound of Formula (I), is characterized below:

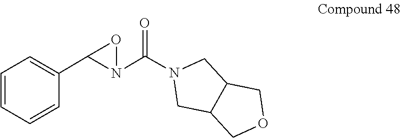

Compound 148. HRMS (ESI) calcd for C₁₄H₁₆N₂O₃ [MH⁺]: 261.1234. Found 261.1504. ¹H NMR (400 MHz, CDCl₃) δ 7.45 (m, 5H), 5.23 (s, 1H), 3.90 (m, 2H), 3.77 (m, 2H), 3.66 (m, 2H), 3.50 (m, 2H), 2.99 (br s, 2H). ¹³C NMR (126 MHz, CDCl₃) δ 160.14, 160.05, 133.08, 133.03, 130.91, 128.79, 128.05, 78.02, 77.96, 73.75, 73.59, 73.46, 73.29, 51.71, 51.44, 51.11, 50.83, 44.29, 44.25, 42.51.

### Example 2: Synthesis of Compound 300

### Synthesis of Intermediate A-2

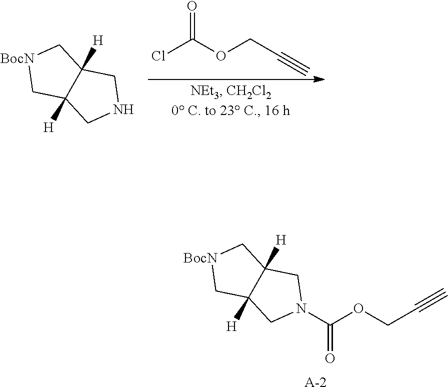

To a stirred solution of amine (3.0 g, 14.1 mmol, 1.0 equiv.) in CH₂Cl₂ (31 mL) at 0° C., was added triethylamine (2.9 mL, 21 mmol, 1.5 equiv.), followed by the dropwise addition of propargylchloroformate (1.5 mL, 15.5 mmol, 1.1 equiv.). The solution was warmed to room temperature overnight. After 16 h, the reaction mixture was diluted with CH₂Cl₂ (50 mL), washed with 1M aq. HCl (20 mL), sat. aq. NaHCO₃ (20 mL), and brine (20 mL). The organic layer was dried over Na₂SO₄, filtered and concentrated in vacuo to afford the desired product as an oil which solidified upon standing to a solid (Intermediate A-2, 4.2 g, >95%). HRMS (ESI): calcd for C₁₅H₂₂N₂O₄Na [M+Na]⁺: 317.1472. Found 317.1479. ¹H NMR (500 MHz, CDCl₃) δ 4.66 (t, J=2.6 Hz, 2H), 3.58 (m, 4H), 3.36-3.13 (m, 4H), 2.83 (s, 2H), 2.44 (t, J=2.4 Hz, 1H), 1.42 (s, 9H). ¹³C NMR (126 MHz, CDCl₃) δ 154.5, 153.9, 79.7, 78.6, 74.6, 52.8, 50.2, 49.7, 49.5, 42.5, 41.6, 40.7, 28.5.

### Synthesis of Intermediate A-3

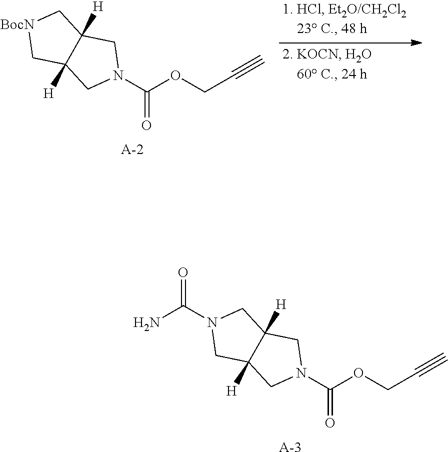

To a stirred solution of Boc-protected amine (Intermediate A-2, 2.0 g, 6.8 mmol, 1.0 equiv.) in CH₂Cl₂ (13 mL) was added HCl (2 M in Et₂O, 12 mL, 24 mmol, 3.5 equiv.) at room temperature. After 48 h, the solvent was removed in vacuo to afford the desired product containing ˜10% remaining starting material. This material was resubjected to the same conditions using HCl (2 M in Et₂O, 6 mL, 12 mmol, 1.8 equiv.). After 16 h, the solvent was removed in vacuo to afford the corresponding amine hydrochloride salt (1.5 g, 96%) as a solid which was directly used in the next step.

To a stirred solution of the amine hydrochloride salt (1.45 g, 6.3 mmol, 1.0 equiv.) in water (6.3 mL) was added KOCN (1.5 g, 19 mmol, 3 equiv.) at room temperature. The reaction was sealed and stirred at 60° C. After 24 h, the reaction was cooled to room temperature and extracted with EtOAc (5×100 mL). The combined organic layers were dried over Na₂SO₄, filtered and concentrated in vacuo to afford the desired urea product (Intermediate A-3, 1.3 g, 88%) as a solid. HRMS (ESI): calcd for C₁₁H₁₆N₃O₃ [M+H]+: 238.1186. Found 238.1199. ¹H NMR (500 MHz, DMSO) δ 5.74 (s, 2H), 4.65 (t, J=2.9 Hz, 2H), 3.53 (m, 2H), 3.48 (t, J=2.4 Hz, 1H), 3.43 (m, 2H), 3.10 (m, 4H), 2.92-2.74 (m, 2H). ¹³C NMR (126 MHz, DMSO) δ 157.4, 153.2, 79.3, 77.3, 52.2, 50.2, 49.6, 49.4, 41.6, 40.6.

### Synthesis of Compound 300

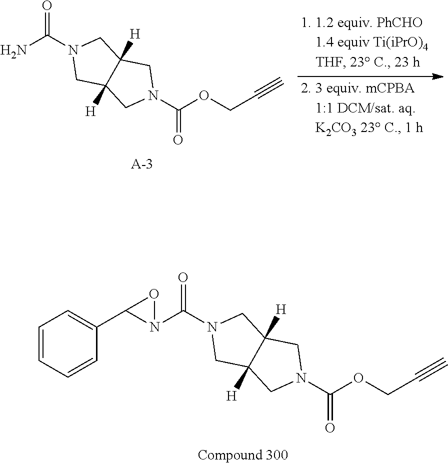 [SMILES: C#CCOC(=O)N1C[C@H]2CN(C(N)=O)C[C@H]2C1 ; "A-3" ; C#CCOC(=O)N1C[C@@H]2CN(C(=O)N3OC3c3ccccc3)C[C@@H]2C1]

To a stirred solution of corresponding urea (Intermediate A-3, 237 mg, 1.0 mmol, 1.0 equiv.) and benzaldehyde (0.12 mL, 1.2 mmol, 1.2 equiv.) in dry THF (3 mL), was added Ti(iPrO)₄ (0.42 mL, 1.4 mmol, 1.4 equiv.) dropwise at room temperature. The mixture was stirred at room temperature for 23 h. The solvent was removed in vacuo to afford crude imine which was used immediately in the next step.

In a 25 mL round-bottom flask, mCPBA (75%, 692 mg, 3.0 mmol, 3 equiv.) was pre-stirred in 1:1 CH₂Cl₂/sat. aq. K₂CO₃ (8 mL) at room temperature for 10 minutes. A solution of crude imine from the previous step (in 1 mL CH₂Cl₂) was added dropwise, using additional CH₂Cl₂ (2×1 mL) rinses for a quantitative transfer. After 1 h, the reaction was diluted with water (30 mL) and extracted with CH₂Cl₂ (3×30 mL). The combined organic layers were washed with brine (30 mL), dried over Na₂SO₄, filtered and concentrated in vacuo. Purification by column chromatography (75% to 85% EtOAc/Hexane) afforded the corresponding oxaziridine (Compound 300, 194 mg, 57%) as a foam. HRMS (ESI): calcd for C₁₈H₂₀N₃O₄ [M+H]+: 342.1449. Found 342.1442. ¹H NMR (500 MHz, CDCl₃) δ 7.44 (m, 5H), 5.22 (s, 1H), 4.78-4.60 (m, 2H), 4.05 (m, 0.4H, minor rotamer), 3.84-3.64 (m, 4H), 3.50-3.29 (m, 3.6H), 3.01-2.87 (m, 2H), 2.46 (m, 1H). ¹³C NMR (126 MHz, CDCl₃) δ 160.4, 153.9, 130.9, 128.8, 128.03, 128.00, 78.5, 78.0, 74.7, 53.0, 50.7, 50.6, 50.1, 50.0, 41.1, 40.1.

### Example 3. Gel-Based ABPP

Activity-based protein profiling (ABPP) was performed as follows. CDK4 was diluted in PBS to 50 nM, then 50 μL was added to each well of a 96-well PCR plate. Ligands were dissolved fresh in DMSO to 5 μM, and 1 μL was added so each well contained a unique ligand at the indicated concentration. Wells were mixed and allowed to incubate 1 h at 23° C. Ligands were then chased with fresh Compound 301 with 1 μL of 5 μM added to each well (100 nM final), wells mixed and allowed to incubate 1 h at 23° C. Excess oxaziridine was quenched with N-acetyl methionine via addition of 1 μL of 10 mM DMSO stock (200 μM final), wells mixed and allowed to incubate 1 h at 23° C. A stock was prepared 1:5 of 12.5 μM DBCO-Cy3 in DMSO: 1.2% SDS/PBS. To each well was added 6 μL of this stock, the wells mixed and allowed to incubate overnight protected from light at 23° C. The next morning 30 μL of 4×Laemmli's buffer containing 10% BME was added to each well. The plate was sealed and brought to 95° C. for 6 min. Samples were loaded and separated on precast 4-20% TGX gels and scanned by ChemiDoc MP for measuring in-gel fluorescence. After that, the total protein level on the gel was assayed by silver staining according to the manufacturer's protocol and scanned by ChemiDoc MP.

### Example 4. Solvent Accessibility

Residue solvent accessibility calculations of methionines on CDK4 protein were computed using the Discovery Studio 2021 platform from Dassault Systemes BIOVIATM. The 2W9Z pdb file for CDK4 was utilized and submitted to a “Solvent Accessibility” calculation. The software was set up with grid points per atom at 240 and probe radius at 1.4 Å.

### Example 5. Shotgun Proteomics

To more accurately assess residue reactivity of oxaziridines on CDK4, 10 μg of CDK4 was diluted with 90 μg whole cell extract derived from mouse liver to a total volume of 100 μL in PBS. Protein mixture was treated with 50 μM oxaziridine (DMSO) and allowed to incubate at 23° C. for 30 min. Labeled protein was precipitated via addition of 900 μL MeOH at −80° C. overnight. The next day, sample was spun at max speed at 4° C. for 10 min. The pellet was gently washed 3 times with a solution of ice cold MeOH. The supernatant was then removed, and the pellet resuspended in 30 μL freshly prepared 8 M urea/PBS. A 5× stock of ProteaseMAX (Promega; cat no. V2071) was prepared by dissolved the pellet in 100 μL ammonium bicarbonate. To the protein mixture was added 30 μL 1×ProteaseMAX, 40 μL ammonium bicarbonate, and 10 μL of 110 mM freshly prepared TCEP (Pierce; cat no. 20490). The sample was then incubated at 60° C. for 30 min. To the sample was then added 2.5 μL of 500 mM freshly prepared iodoacetamide (Sigma Aldrich; cat no. I1149), and the sample was incubated protected from light at 23° C. for 30 min. 120 μL of PBS was then added, followed by 1.2 μL 5×ProteaseMAX. The sample was vortexed thoroughly. Sequencing grade Trypsin/Lys-C mix (Promega; cat no.V5071) was reconstituted in 40 μL trypsin buffer, and 4 μL was added to the sample. The sample was allowed to digest at 37° C. overnight. The next day the sample was acidified with 12 μL formic acid and spun at max speed for 30 min. The supernatant was taken to a low-adhesion tube and stored at −80° C. until MS analysis, as outlined in Example 6.

### Example 6. Mass Spec Analysis

Peptides from all experiments were analyzed on a Q Exactive Plus mass spectrometer (Thermo Fisher Scientific); pure proteins were analyzed on a C18 only column, while complex proteomes were analyzed via a five step Multidimensional Protein Identification Technology (MudPIT).⁷ In both cases, Inlines (IDEX; cat no.M-520) were fitted with ˜20 cm of 250 μm tubing and prepacked with 4 cm of Aqua C18 reverse-phase resin (Phenomenex; cat no. 04A-4299). Columns were made from 100 μm tubing pulled with a P-2000 laser (Sutter Instruments Co.) and packed with either 13 cm of Aqua C18 reverse-phase resin (pure proteins) or packed first with 10 cm of Aqua C18 reverse-phase resin followed by 3 cm of strong-cation exchange resin (Phenomenex; cat no.04A-4398; MudPIT). Both inlines and columns were equilibrated after packing prior to use on an Agilent 1260 HiP AlS coupled to an Agilent 1260 Quat Pump using the following gradient: 100% buffer A to 100% buffer B over 10 min, hold at buffer B for 5 min, finish with a wash with 100% buffer A over 15 min (where buffer A is 95:5 H₂O:MeCN/0.1% formic acid, and buffer B is 80:20 H₂O:MeCN/0.1% formic acid). For best performance, columns were equilibrated the day of sample run.

Peptides were pressure-loaded onto an equilibrated inline until dry. The tubing was cut to ˜2 cm above the resin bed and an appropriate equilibrated column was attached to the opposite end of the inline. The sample was attached to the LC-MS/MS via a MicroTee PEEK 360 m fitting (Thermo Fisher Scientific; cat no. p-888) and the column tip aligned with the MS source opening. Data was collected in positive-ion mode using data-dependent acquisition mode with dynamic exclusion enabled (60 s) between 400 and 1800 m/z and a mass resolution of 70,000, with one MS1 scan followed by 15 MS2 scans of the nth most abundant ions. Nanospray voltage was set at 2.75 kV and heated capillary temperature at 200° C. The MudPIT program utilized for all samples consists of five separate programs run sequentially, where each begins with either 0, 25, 50, 80, or 100% salt bump (buffer C, 500 mM ammonium acetate/H₂O) followed by a gradient of 5-55% buffer B in buffer A. Pure proteins were run on only the first program (0% salt bump) from the MudPIT program. The flow was kept at 0.1 mL/min throughout.

Data were analyzed with Byologic (Protein Metrics Inc.). Raw files were searched directly against the Uniprot human or mouse database using the Byos HCP workflow, with decoys and common contaminants added. Peptides were assumed fully tryptic. All searches included the following modifications: Acetyl (+42.010565; Protein N-term; variable—rare1), carbamidomethyl (+57.021464; C; Fixed), and oxidation (+15.994915; M; variable—common1). Other modifications to methionine depended on the probe added to the sample and were all treated as variable—common1: Compound 300 (+235.09569), Compound 148 (+154.07423), Compound 160 (+168.08988). Peptides with b and y ions flanking the site of modification were assumed modified.

### Example 7. Identification of Ligandable Methionine Sites on CDK4

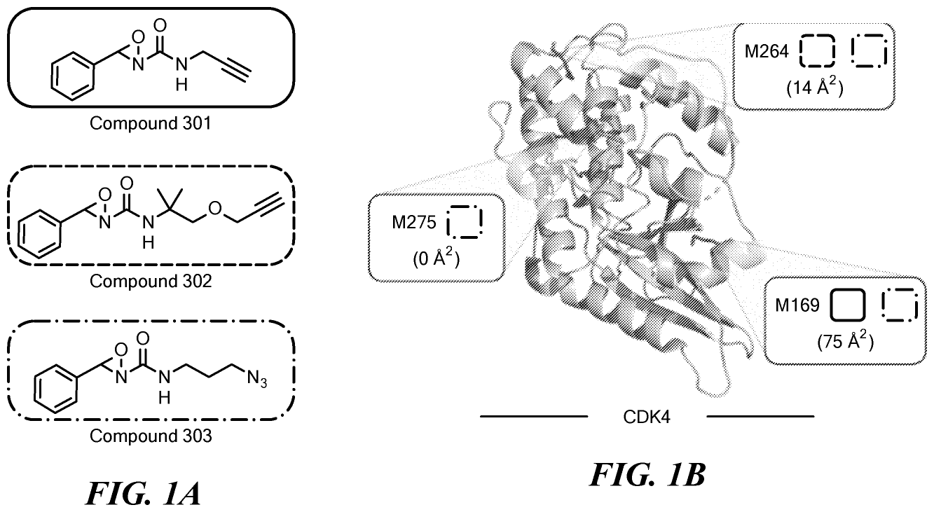

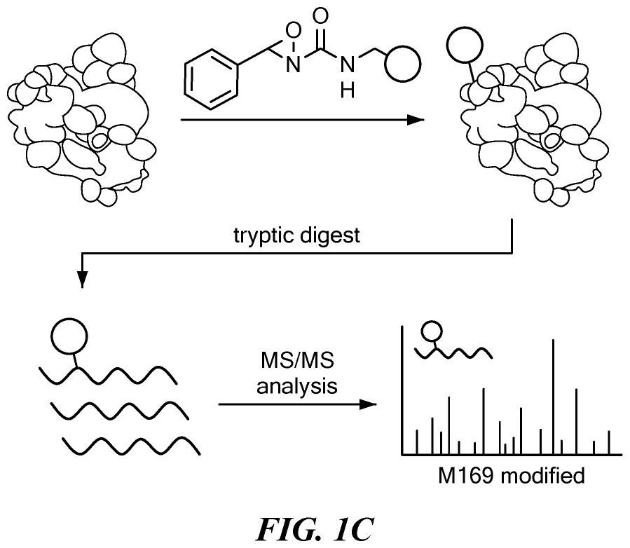

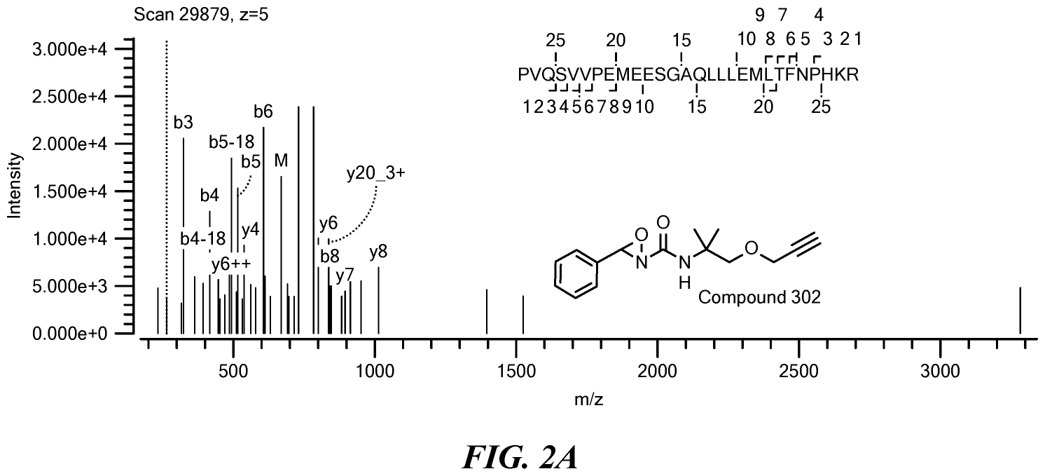

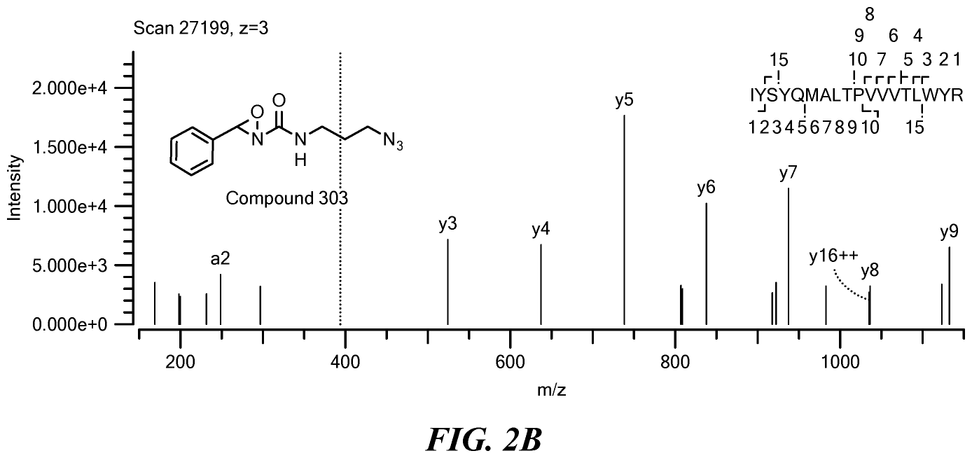

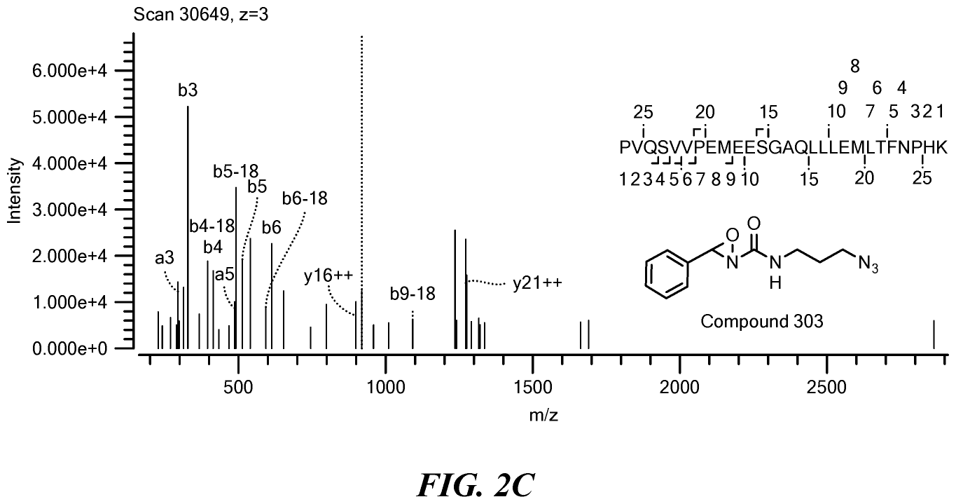

CDK4 was selected as a representative kinase for the starting point to develop a methionine-targeting platform for covalent ligand discovery. To identify potential new ligandable methionine sites in CDK4, three unique oxaziridine probes were applied to this target: Compounds 301, 302, and 303, using the gel-based ABPP methods outlined in Example 3, as shown in FIG. 1A. Each oxaziridine probe showed a different pattern of covalent methionine labeling on the CDK4 target, suggesting that these sites can be preferentially targeted. As shown in FIG. 1B and FIG. 2, Compound 303 modifies three reactive sites: M169, M264, and M275. In contrast, Compound 301 labeled only M169, whereas Compound 302 engaged only M264. Sites of modification were determined via shotgun proteomics, as described in Example 5, shown in FIG. 1C. The frequency of modifications observed on each methionine site correlated with the solvent accessibility of that residue, as described in Example 4. M169 displayed the highest reactivity, followed by M264, with M275 being the least solvent accessible and least reactive. The newly identified reactive M169 site is proximal to a known T172 phosphorylation site on CDK4 that activates protein function.

### Example 8. Design and Synthesis of a Methionine-Targeting Covalent Ligand Library

Development of a methionine-directed covalent ligand platform to engage the reactive methionine residues of CDK4 was sought through the design and synthesis of a focused oxaziridine fragment library. A suite of synthetic routes to such products was optimized, which included three methods for imine synthesis followed by oxidation via mCPBA, as seen in Example 1, and generally in Scheme A. As such, an oxaziridine library to achieve sufficient structural diversity was prepared. The design of the oxaziridine library was initiated through a triaging selection of the key diversity element, namely the corresponding amine component. 43,950 amines were available for selection (Enamine); utilizing ICM Chemist Pro (Molsoft LLC) sub-structure selections was made: azetidine (286), pyrrolidine (2711), piperidine (3889), cycloheptylamines (328), iso-propylamines (4452), iso-butylamines (627), morpholines (409), ethylamine (6057), cyclo-heptylamines (328), 2,3,4,5-tetrahydro-1H-benzo[d]azepine (19) and iso-indolene (37). These choices were driven by oxaziridine reagents that produced more stable methionine adducts.

TABLE 4
Diversity of amine element of the oxaziridine library and clustering analysis.

| Chemical sub- | Number of | Number of | Tanimoto | Compounds | |
|---|---|---|---|---|---|
| structure | derivatives | initial clusters | coefficient | per cluster | Selected |
| Azetidine | 286 | 80 | 0.37 | 1-41 | 28 |
| Pyrrolidine | 2711 | 524 | 0.3 | 1-61 | 64 |
| Piperidine | 3889 | 595 | 0.3 | 1-138 | 19 |
| Ethyl amine | 6057 | 1199 | 0.3 | 1-142 | — |
| Iso-butyl amine | 627 | 142 | 0.3 | 1-45 | 28 |
| Iso-propyl amine | 4452 | 672 | 0.3 | 1-175 | 17 |
| Morpholine | 409 | 75 | 0.3 | 1-61 | 36 |
| Cycloheptyl amine | 328 | 96 | 0.3 | 1-45 | 29 |
| Iso-indoline | 37 | 16 | 0.20 | 1-7 | 9 |
| 2,3,4,5-tetrahydro- | 19 | 9 | 0.25 | 1-6 | 4 |
| 1H-benzo[d]azepine | | | | | |

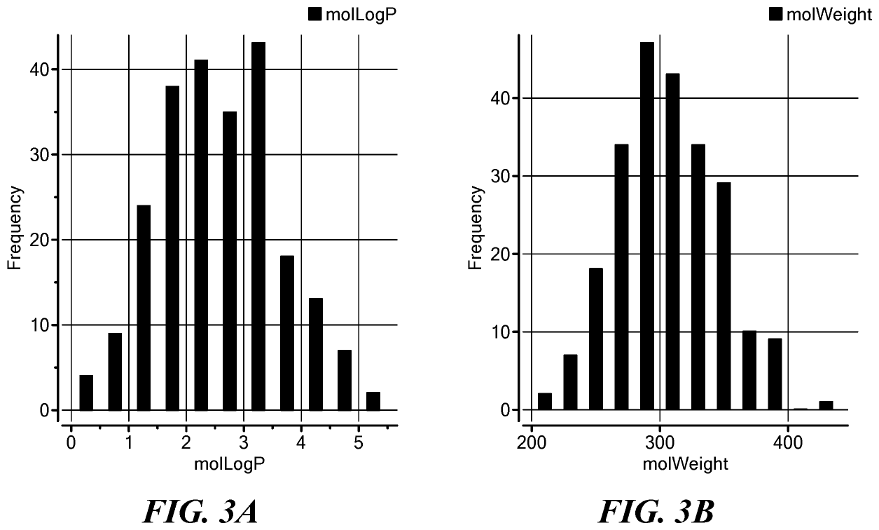

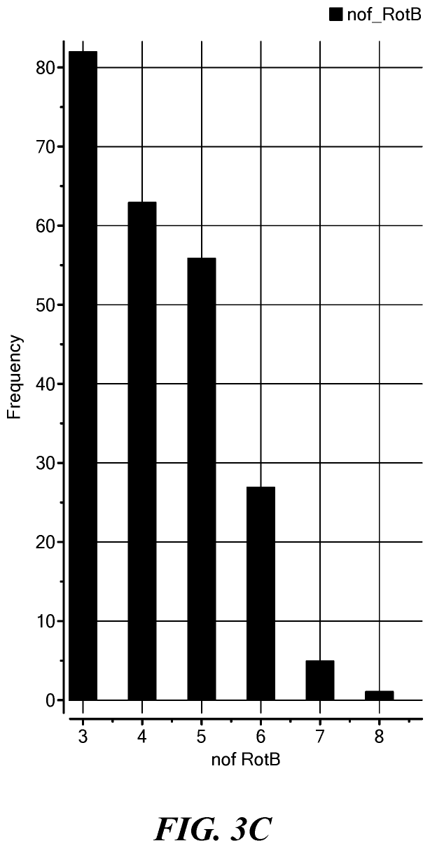

Clustering analysis for each selected amine set was conducted using ICM Chemist Pro (Molsoft LLC) using (Tanimoto <0.4 as noted) generating the detailed clusters for each sub-type. Thereafter, two rounds of selections based on diversity and selecting the square root of the population of each cluster were conducted followed by a visual inspection to remove compounds containing foreseen chemoselectivity issues, based reactivity with mCPBA. The resulting composite set was stripped of duplicate selections from within the sub-sets and the resultant 234 amines were then derivatized to the corresponding oxaziridine (from benzaldehyde), with the log P and MW distributions, shown in FIG. 3A-B, for the products; the library also was analyzed showing all compounds with HBD≤1, the number of rotatable bonds was ≤8 with the majority being 3-5, and tPSA distribution was 27-78A, shown in FIG. 3C. These properties were not constraints for the library as the initial selections were made for structural diversity. All compounds were then selected for conformational analysis.

### Example 9. Conformational Analysis

Each of the 234 structures from previous Example 8, were coded as SMILES, converted to 3D using Corina with the following flags “-d wh,stergen,axchir,preserve,msc=2”.³ Symmetry equivalents were removed, then conformers were constructed using omega2[omega2] on default settings. The conformers were optimized and IR frequencies computed using Gaussian09[G09] with the keywords “opt(maxcycle=250) freq pop=nbo def2tzvp m062x”.⁵ For each conformer, the most intense frequency (F) between 1600.0 and 1740.0 cm⁻¹ for the sulfimines, and 1700.0 and 1950.0 cm⁻¹ for ureas, and the sum of the electronic and thermal Free Energies, were collected. The final figures are derived from a Boltzmann weighted average of all the conformers of a structure.

### Example 10. Automated Parameter Extraction

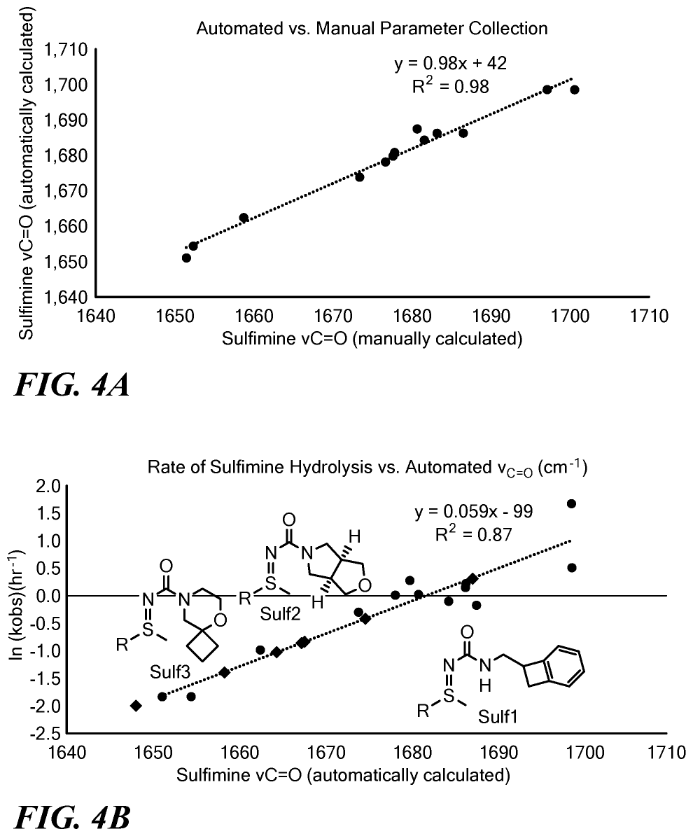

The automated computational workflow⁶ was validated by obtaining the stretching frequencies from the set of compounds used to generate the reported hydrolysis kinetic model,² and compared to the frequencies obtained manually, shown in Table 5, below. It was found that the automatically calculated stretching frequencies correlated very strongly with those obtained manually, as shown in FIG. 4A-B, with a slope and R²=0.98. As a further point of validation, the published rates of hydrolysis were plotted against the automatically calculated stretching frequency, which produced a similar model to the one obtained from manual calculated stretching frequencies. With this validation in hand, the stretching frequencies of a novel library of untested oxaziridines were extracted, to predict their respective sulfimine adduct stabilities towards hydrolysis. The predicted K_{obs} are computed as 0.0595*(νC═O)−112.8 for sulfimines, and 0.0498*(νC═O)−102.8 for ureas. Equivalent structures in the two series are paired up in the output. Of the 234 input pairs, 1 sulfimine and 1 urea failed to converge, even after restarting, possibly due to unresolved steric crowding.

TABLE 5
Sulfimines used for validating the automated workflow for obtaining C═O
stretching frequencies. Manually calculated parameters and rate of hydrolysis obtained from
previous report (Christian, A. H., et al;. J. Am. Chem. Soc. 2019, 141 (32), 12657-12662;
*denotes predicted hydrolysis rates).

| Sulfimine Structure 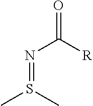 | R-group | Manual Collection | Automated Collection | Rate of Hydrolysis (In(k_{obs})) (hr⁻¹) |
|---|---|---|---|---|
| 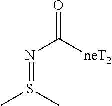 | —NEt₂ | 1652.31 | 1654.42 | −1.82 |
| 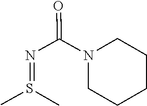 | Piperidine | 1651.49 | 1651.09 | −1.81 |
| 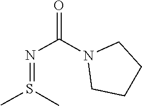 | Pyrrolidine | 1658.72 | 1662.44 | −0.96 |
| 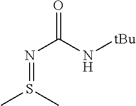 | —NHtBu | 1673.34 | 1673.81 | −0.28 |
| 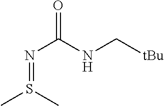 | —NHCH₂tBu | 1680.60 | 1687.47 | −0.15 |
| 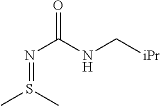 | —NHCH₂iPr | 1681.51 | 1684.25 | −0.085 |
| 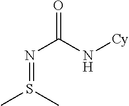 | —NHCy | 1676.60 | 1678.08 | 0.032 |
| 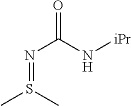 | —NHiPr | 1677.78 | 1680.79 | 0.044 |
| 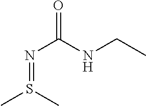 | —NHEt | 1683.11 | 1686.17 | 0.17 |
| 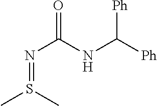 [SMILES: CS(C)=NC(=O)NC(c1ccccc1)c1ccccc1] | —NHCHPh₂ | 1686.45 | 1686.23 | 0.24 |
| 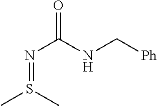 | —NHBn | 1677.57 | 1679.74 | 0.29 |
| 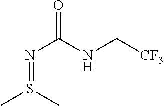 | —NHCH₂CF₃ | 1697.01 | 1698.51 | 0.53 |
| 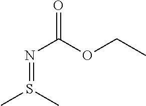 | —OEt | 1700.52 | 1698.47 | 1.69 |
| 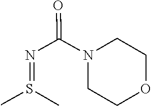 | Morpholine | 1659.09 | 1664.30 | −1.02* |
| 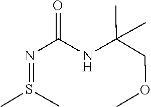 | —NHCMe(CH₂OMe)₂ | 1666.60 | 1667.23 | −0.85* |
| 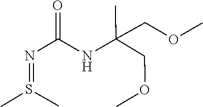 | —NHC(CH₂OMe)3 | 1667.09 | 1667.55 | −0.83* |
| 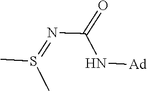 | —NHAd | 1671.38 | 1674.58 | −0.41* |
| 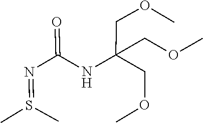 | —NHCMe₂CH₂OMe | 1675.08 | 1674.60 | −0.41* |

Ad = Adamantyl

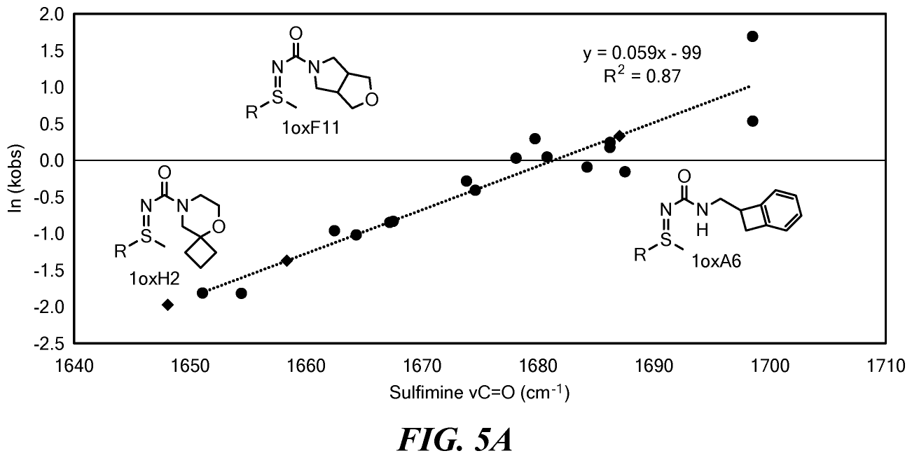

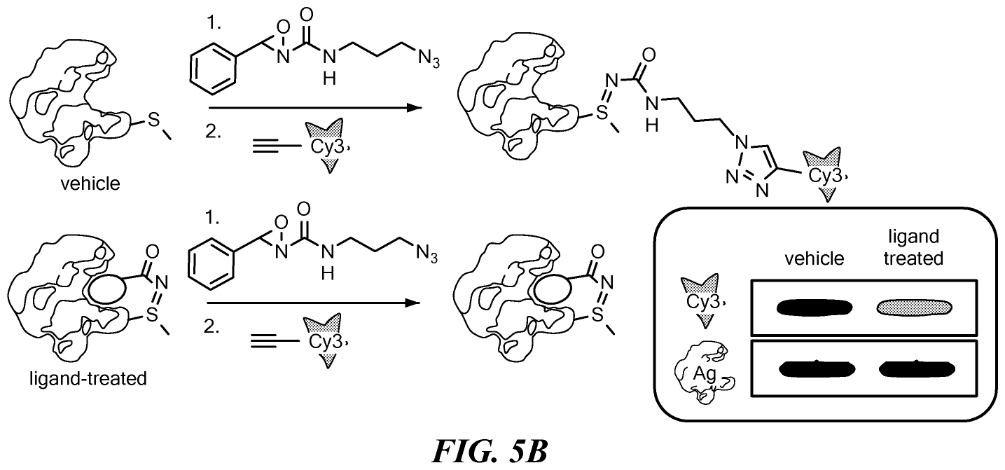

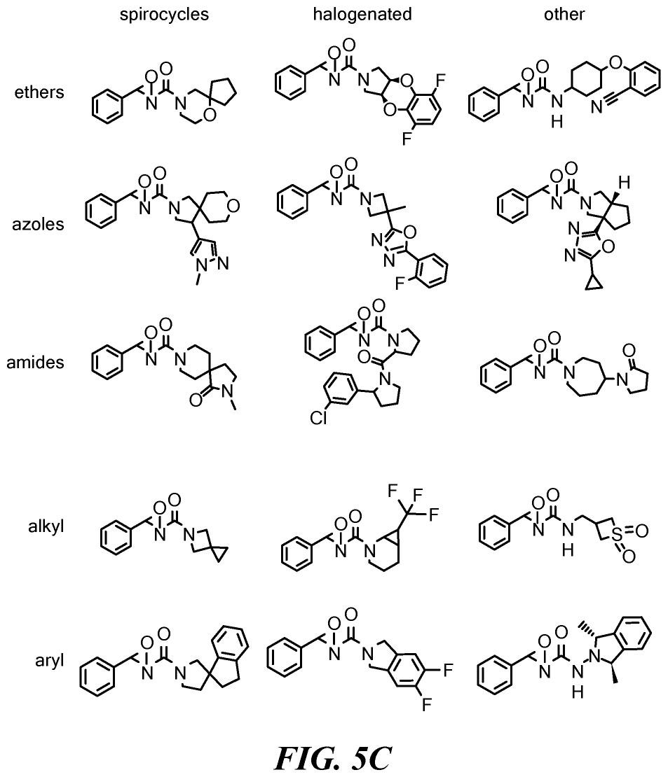

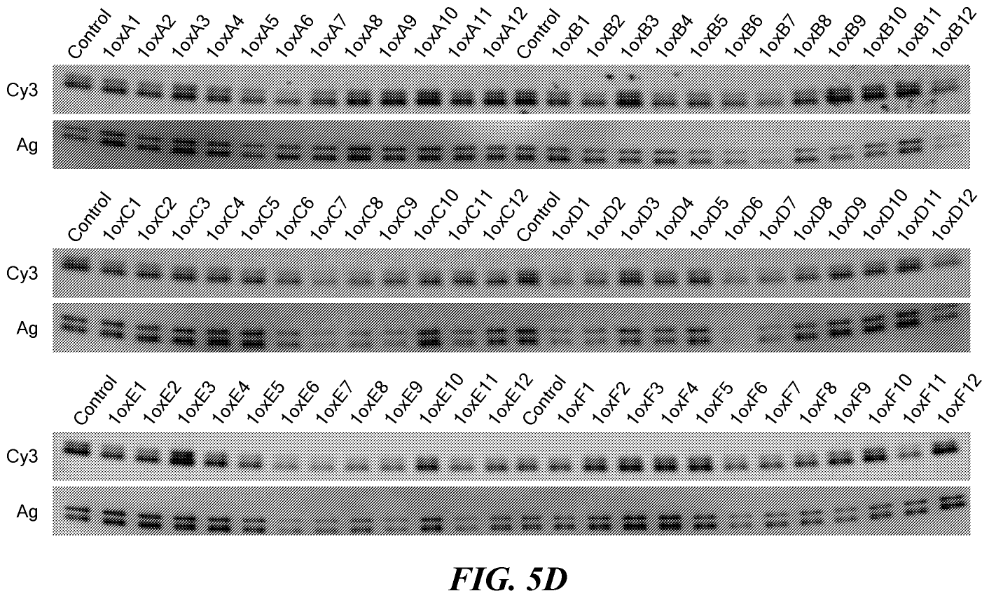

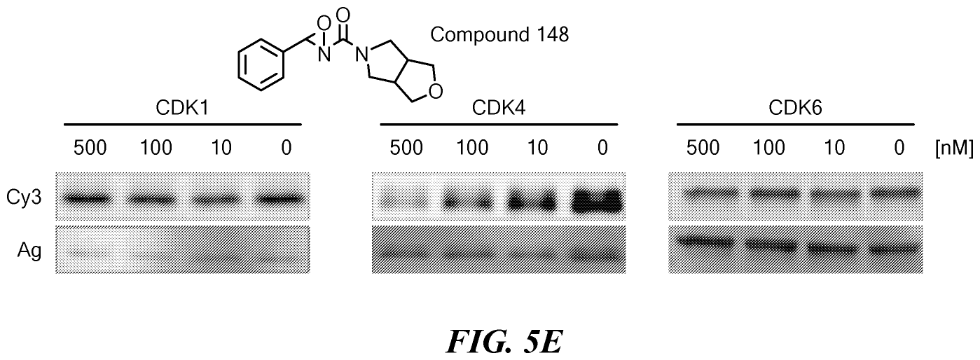

The amines were derivatized to the corresponding sulfimine or urea in silico and a 3-D conformational search allowed C═O stretching frequencies to be determined. Principle component analysis (PCA) methods were employed to optimize and ensure formation of N-transfer sulfimine over O-transfer sulfoxide products upon reaction with methionine, and found that rates of sulfimine hydrolysis correlated with the calculated sulfimine adduct νC═O values, as shown in FIG. 5A. The finalized library of 179 unique oxaziridine fragments featured a diverse array of functional groups, including spirocycles, halogens, azoles, ethers, and amides, as seen in Tables 1, 2, and 3.

### Example 11. Methionine-Directed ABPP Screen Against CDK4 to Identify Key Compounds

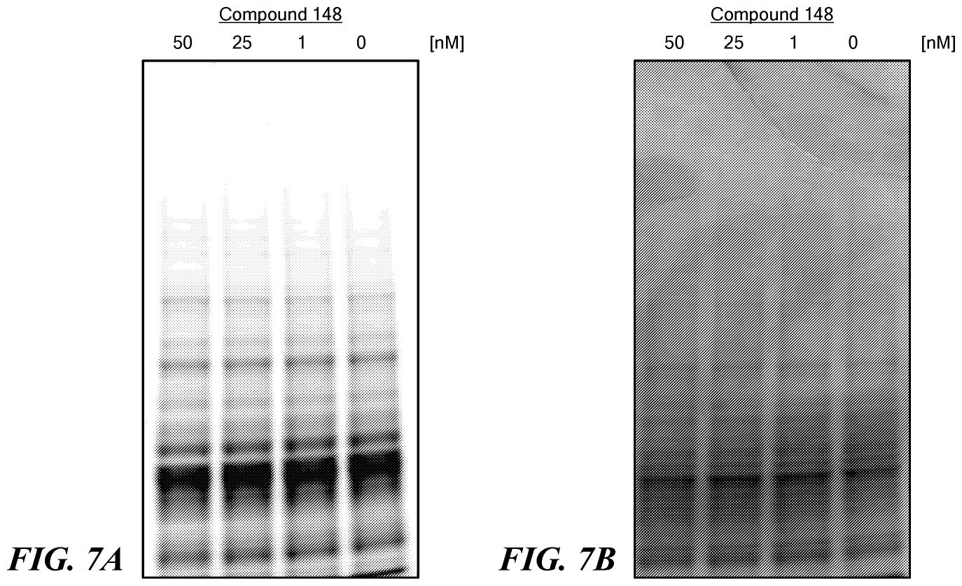

The oxaziridine fragment library from Example 8 was screened for methionine-directed modifiers of CDK4 via a gel-based ABPP platform as described in Example 3, seen in FIG. 7B. Compound 303 was chosen, as it was the most promiscuous and could engage three reactive methionine sites in competitive binding assays with potential covalent oxaziridine ligands. Isolated CDK4 was treated either with DMSO (vehicle) or a covalent ligand from the oxaziridine library (ligand-treated). Samples were then treated with Compound 303, followed by a quench step with N-acetyl methionine (NAM) to remove any excess oxaziridine. DBCO-Cy3 was then introduced by strain-promoted click chemistry to provide a fluorescence readout. The samples were subsequently separated via SDS-PAGE and fluorescence signals were normalized via silver stain to triage any covalent ligands that induced general protein aggregation, which would generate a false positive Cy3 signal, as shown in FIG. 5B. The library contained fragments bearing a variety of functionalities, including spirocycles, halogens, azoles, ethers, and amides, as shown in FIG. 5C.

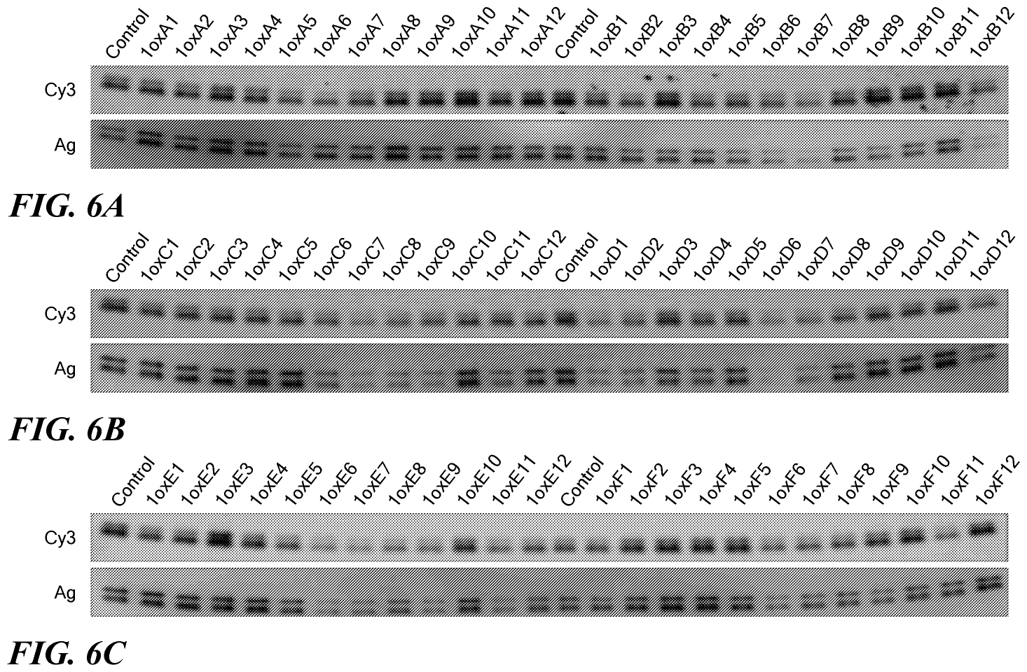

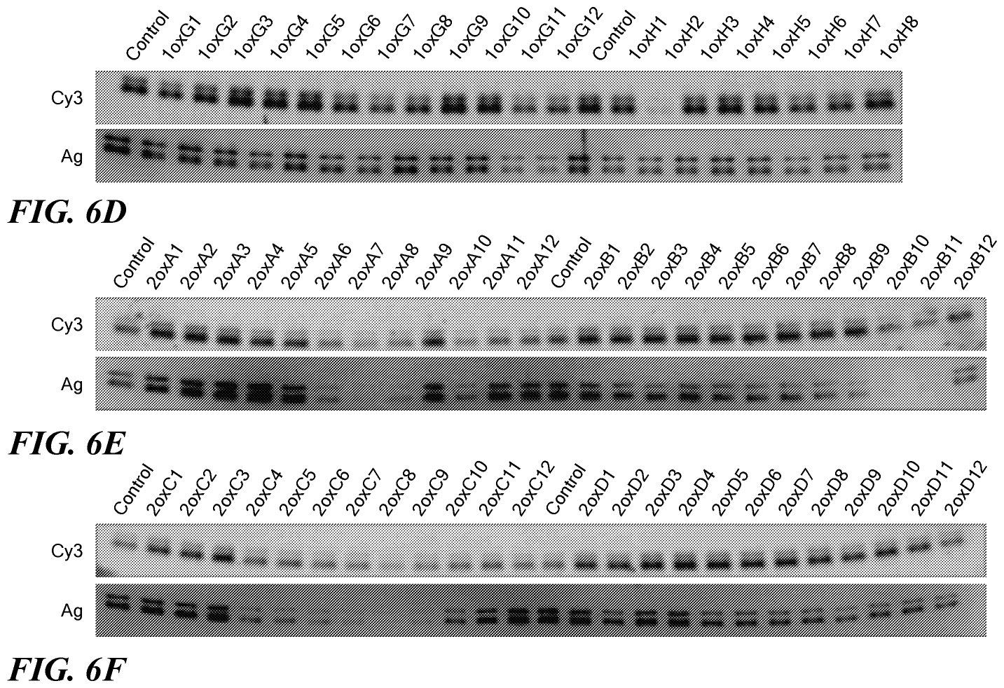

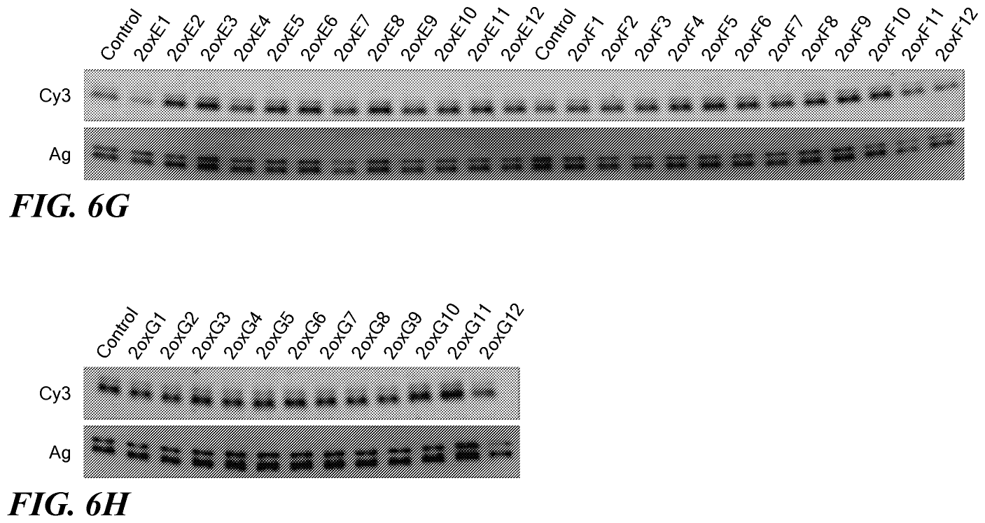

Using ReACT, ABPP screening, as outlined in Example 3, of the focused oxaziridine fragment library from Example 8 on CDK4 revealed Compound 148 as a candidate for further study, seen in FIG. 5D and FIG. 6A-H. This fragment showed competition with the Compound 303 probe for CDK4 binding in a dose-dependent manner. The isoform specificity was tested with two closely-related congeners, CDK1 and CDK6. It was observed that Compound 148 displayed no dose-dependent labeling against either CDK1 or CDK6 by gel analysis, as seen in FIG. 5E. It was confirmed that Compound 148 did not induce general aggregation in cell lysates, as shown in FIG. 7.

### Example 12. Compound 148 is a Covalent Modifier of CDK4 at the M169 Site and Inhibits Activity on Purified Protein

The interaction between Compound 148 and CDK4 was then characterized in vitro. To start, shotgun proteomics was performed with Compound 148 on purified CDK4, CDK1, and CDK6 proteins, as described in Example 5.

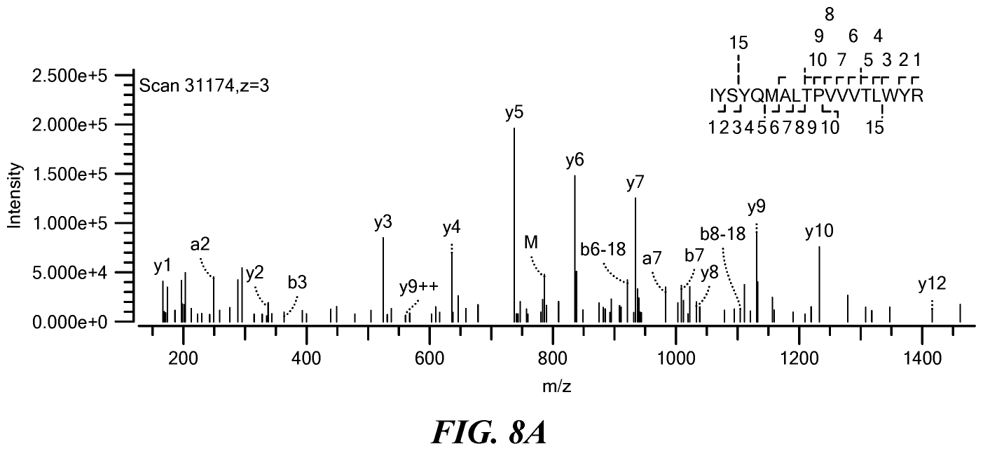

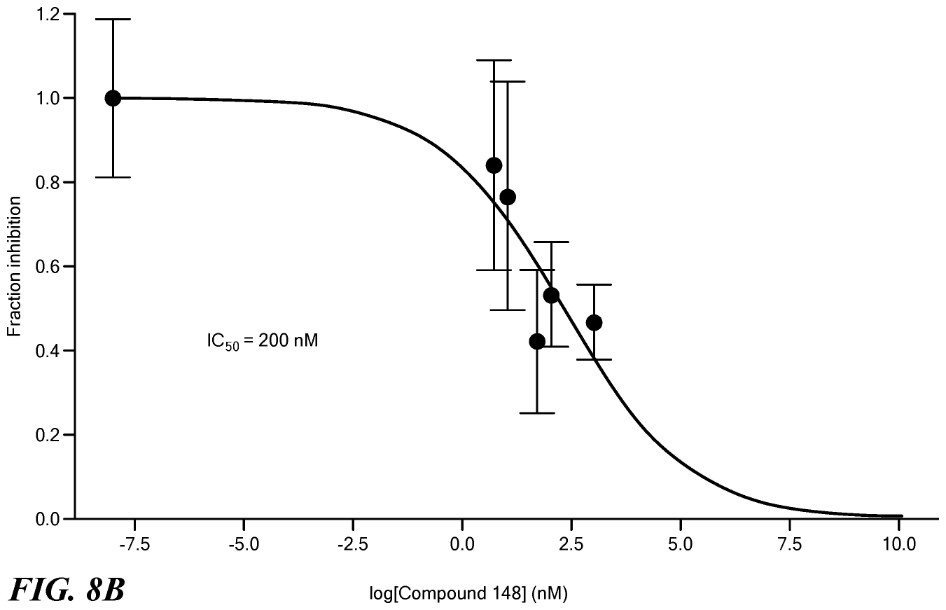

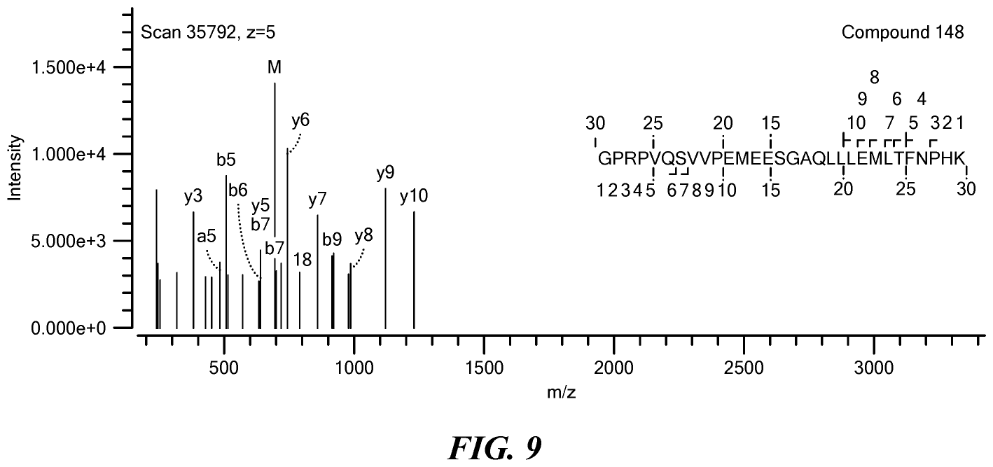

The primary site of modification on CDK4 by Compound 148 was identified to be M169, with minor labeling at M264, as shown in FIG. 8 and FIG. 9. No modifications by Compound 148 were observed on either CDK1 or CDK6, further supporting the isoform specificity of this oxaziridine. Compound 148 was shown to inhibit activity of purified CDK4 protein in a dose-dependent manner using a luciferase-based activity assay as a proxy for kinase activity, observing an IC₅₀ around 200 nM, as demonstrated in FIG. 8B.

### Example 13. In Vitro Activity Assay

Effects of Compound 148 on in vitro activity of CDK4 were determined via commercially available ADP-Glo assay (Promega; cat no.V6930). Isolated CDK4 was provided by Novartis. Retinoblastoma protein (aa 773-928) was obtained from commercial sources (Millipore Sigma; 12-439). The assay kit protocol was followed as directed, with the addition of a quench step (addition of 1 μL of 25 μM N-Acetyl methionine) after incubation of protein with oxaziridine ligand.

### Example 14. Cell Culture

All cells were maintained as a monolayer in exponential growth at 37° C. in a 5% CO2 atmosphere. MCF-7 and HepG2 were maintained in Dulbecco's Modified Eagle Medium (DMEM, Gibco) supplemented with 10% (v/v) fetal bovine serum (FBS, Seradigm). HT-29 and SW-48 were maintained in RPMI 1640 Medium (Gibco) supplemented with 10% FBS.

### Example 15. Cell Synchronization

Cells were synchronized to G₁ via serum starvation. Cells were plated at 50% confluency and allowed to adhere overnight in serum-containing media. The next morning, the cells were gently washed twice with HBSS, and serum-free media was added. Cells were allowed to incubate an additional 24 h, for a total of 48 h without serum. Final cell confluency should remain below 75% for optimal results.

### Example 16. Cell Treatment

All cells dosed with oxaziridine were treated using a 2% DMSO/media solution. Oxaziridines were dissolved in DMSO and used the same day as treatment. Media was removed from cells and replaced with fresh media containing oxaziridine in DMSO for a final concentration of 2% DMSO. Control wells were treated with 2% DMSO/media. Cells were incubated at 37° C. in a 5% CO₂ atmosphere.

### Example 17. Cell Viability Assay

Commercially available Cell Counting Kit-8 Assay (Dojindo) was used to investigate cell viability after treatment with oxaziridines. Cells were plated in 96-well plates (black/clear bottom; Thermo Fisher) and grown to 75% confluency. Media was removed and replaced with media containing 2% DMSO and indicated compound concentration. Cells were allowed to incubate 24 h. To assess viability, media was removed and replaced with 100 μL media containing 10% CCK-8 assay solution. Plates were incubated at 37° C. in a 5% CO₂ atmosphere for 1-4 h until an orange color was visible. Viability was quantified via plate reader (monofilter) with absorption at 450 nm.

### Example 18. Western Blot Analysis

Cells were seeded in full media at 8 e5 cells/well in a 6-well plate. They were then serum-synchronized, as outlined in Example 15, and treated with compound as described previously. Cells were monitored by eye for cell death and harvested once high-dose cells were round but not detached. Cells were transferred to ice and washed twice gently with 1 mL ice-cold PBS. 10 mL of lysis buffer was prepared by dissolved a protease-inhibitor table (Pierce; cat no. A32953) and a phosphatase-inhibitor tablet (Roche; cat no. 4906845001) in 10 mL PBS (1% Triton X-100). Cells were scraped to harvest, transferred to 1.5 mL Eppendorf tubes, and incubated at 4° C. for 30 min on a rotator. Samples were spun at 5000×g for 10 min to clarify lysate. Supernatant was transferred to a new tube and protein concentration quantified via BCA assay (Pierce; cat no. 23225). Samples were normalized to the lowest concentration using chilled lysis buffer. Samples were diluted with 4×Laemmli's buffer (10% BME) (Bio-Rad; cat no. 1610747) and loaded at 25 μg per lane on a 4-20% Tris-Gly SDS-PAGE gel. The gel was run at 160 V for 80 min and semi-dry electrotransferred to a PVDF membrane at 25 V, 2.5 Å, for 10 min. Blots were blocked with 5% BSA/TBST for 1 h, then washed 2×TBST for 5 min, and cut for incubation with separate antibodies. Antibodies used were rabbit anti-pRb Ser807/811 (CST; 9308), rabbit anti-pRb Ser780 (CST; cat no. 3590), rabbit anti-β actin (CST; cat no. 4970), mouse phospho-T172 CDK4 (NB8-AD9), and rabbit anti-CDK4 (CST; cat no. 12790). All antibodies were diluted at 1:1000 in 5% BSA/TBST, with the exception of AD9 diluted at 1:500, at 4° C. overnight. The next morning the blots were washed 3× with TBST prior to incubation with anti-rabbit IgG HRP conjugated secondary (CST; cat no. 7074) (1:3000 TBST) or anti-mouse IgG HRP conjugated secondary (CST; cat no. 7076S) (1:3000 TBST) for 2 h at room temp. Blots were quickly washed 3× with TBST prior to incubation with ECL western blotting substrates (Promega; cat no. W1001) for 1 min and imaging with ChemiDoc MP.

### Example 19. 2D Gel Electrophoresis

Serum-synchronized MCF-7 cells were treated with compound according to method outlined. Cells were monitored by eye for cell death and harvested once high-dose cells were round but not detached, about 2 hours. Media was removed and cells washed with HBSS. Trypsin was added to detach cells. Cells were transferred to a falcon tube containing 7 mL of complete DMEM and centrifuged at 1200 rpm for 2 min. Supernatant discarded and pellet gently resuspended in 1 mL PBS and transferred to an Eppendorf tube. Sample was spun once more, after which the pellet was quickly rinsed with ice cold MQ and spun a final time. Pellet was aspirated, flash frozen with LN2, and stored at −80° C.

SCell pellets were solubilized in cold 30 mM Tris buffer pH 8.5 containing 7 M urea, 2 M thiourea and 4% CHAPS with continuous vortexing until unfrozen and then kept agitated for 20 min. After centrifugation at 15,700 g for 10 min at 4° C., proteins were quantified. An equal volume of 2-D-sample buffer (7 M urea, 2 M thiourea, 2% CHAPS, 0.4% 3-10 Pharmalytes, and 0.4% DTT) was added to samples normalized to 150 μg proteins. Proteins were separated by isoelectrofocusing on immobilized linear gradient (pH 5 to 8 [11 cm], Bio-Rad) strips, separated by SDS-PAGE and immunoblotted with antibodies against CDK4 (D9G3E, rabbit monoclonal) or phospho-T172 CDK4 (NB8-AD9, mouse). Secondary antibodies were coupled to horseradish peroxidase (Cell Signaling Technology). The proteins were detected using Western Lightning Plus ECL (Perkin Elmer) and viewed in Fusion FX gel documentation system using the Solo7S camera (Vilber Lourmat, France).

### Example 20. Expression of CDK4 in MCF-7 Cells

MCF-7 cells were grown to 40% confluency in 3 mL DMEM media (Gibco) containing 10% (v/v) FBS in a 6-well chamber at 37° C., 5% CO₂. Transfection was then performed as per Lipofectamine 2000 protocol (Invitrogen). Briefly, 0 or 2.5 μg of pcDNA3.1(+)-FLAG-TEV-CDK4 expression construct was introduced at 0:0, 2:1 and 3:1 transfection reagent:DNA. The lipid-DNA complex was incubated for 30 mins at 23° C. in Opti-MEM media (Gibco). Then, 250 μL complex was added to 2.75 mL DMEM containing no FBS. 3 mL DMEM+DNA was added to each well, while control wells received DMEM only. Cells were incubated for 6H at 37° C., 5% CO₂. The media was then aspirated, and cells were given 3 mL DMEM+10% FBS and left to incubate for an additional 30H at 37° C., 5% CO₂. Cells were washed 3× in 500 μL ice-cold PBS then harvested by scraping in 200 μL ice-cold PBS+1% Triton X-100 containing EDTA-free protease inhibitors (Roche). Cells were lysed at 4° C. for 30 mins while rocking. Lysate was clarified by centrifugation at 10,000×g at 4° C. for 15 mins. The supernatant was then transferred to a fresh prechilled 1.5 mL microcentrifuge tube. Protein concentration was normalized to 2.0 mg/mL via BCA assay (Pierce; cat no. 23225). Samples were denatured in 4×Laemmli's buffer+10% BME (Bio-Rad; cat no. 1610747) and boiled at 95° C. for 8 mins. 30 μg protein was loaded onto a 4-20% Tris-Gly SDS-PAGE gel run at 160 V for 70 mins. Proteins were then electro-transferred to a PVDF membrane (25 V/2.5 Å for 10 mins). Membranes were then blocked in a solution of TBST+5% BSA (w/v) and rocked at 23° C. for 1H. Membranes were washed 3× in TBST for 5 mins each while rocking and cut using a razor blade along the protein ladder for separate antibody incubation. Membranes were then blotted with primary rabbit anti-CDK4 (CST; cat no. 12790) or rabbit anti-β actin (CST; cat no. 4970) (both as 1:1000 TBST+5% BSA suspensions) at 4° C. overnight. The following morning membranes were washed 3× in TBST for 5 mins then incubated with anti-rabbit IgG HRP conjugated secondary (1:3000 TBST) (CST; cat no. 7076S) for 2H at 23° C. Blots were quickly washed 3× with TBST prior to incubation with ECL western blotting substrates (Promega; cat no. W1001) for 1 min and imaging with ChemiDoc MP. CDK4 expression was quantified by densitometry (ImageJ) and normalized to the actin loading control.

### Example 21. Competition and Pulldown of Compound 300/Compound 148

MCF-7 cells were expressed with CDK4-TEV-FLAG and normalized to 2 mg/mL as described. To 50 μL of this lysate was added 1 μL of a stock of Compound 148 in DMSO for a final concentration of 500 μM, 250 μM, or 0 μM Compound 148. A sample was set aside as “just lysate,” which was not treated with any compounds. After incubation with Compound 148 for 1 hour at 22° C., 1 μL of 25 mM Compound 300 was added to each sample as indicated for a final concentration of 500 μM Compound 300. Samples were incubated again for 1 hour at 22° C. DTB-N₃ was added to each compound with TBTA, TCEP, and Cu₂(SO₄), and Copper-catalyzed azide-alkyne cycloaddition (CuAAC) was allowed to proceed for 1 h at 22° C. 450 μL of MeOH was added to each sample and proteins were precipitated at −80° C. for 12 h. Samples were then spun at max speed for 10 min at 4° C. Pellet was resuspended in cold MeOH and samples spun again. Pellet was washed once more before resuspension in 150 μL 0.2% SDS/PBS. Samples were then boiled for 5 min and spun at 6500×g for 5 min. Meanwhile, high-capacity streptavidin beads (10 μL per sample) (Thermo Fisher Scientific; cat no. 20357) were washed 2× in PBS and 2× in MQ. The supernatant of the samples was added to 10 μL of the washed beads and allowed to incubate at 4° C. overnight. The next morning the samples were allowed to rock at 22° C. to resolubilize the SDS. The supernatant was collected and set aside for analysis. The beads were washed thoroughly with 2×PBS and 2×MQ and transferred to micro bio-spin columns (Bio-Rad; cat no. 7326204). Peptides were eluted from beads via addition of 2×75 μL 0.1% FA (50% MeCN/MQ). Eluent was collected and beads washed once more with 20 μL of elution buffer. Columns were spun at 3000×g for 3 min. Eluent was lyophilized to remove MeCN. After samples were dry, they were reconstituted in 75 μL PBS. Protein was diluted with 4×Laemmli's buffer (Bio-Rad Laboratories, Inc.; cat no. 1610747) containing 10% BME and brought to 95° C. for 6 min. Samples were loaded and separated on precast 4-20% TGX gels (Bio-Rad Laboratories, Inc.). The gel was run at 160 V for 80 min and semi-dry electrotransferred to a PVDF membrane at 25 V, 2.5 Å, for 10 min. A separate gel was run and stained for total protein via Coomassie. Blot was blocked with 5% BSA/TBST for 1 h, then washed 2×TBST for 5 min. Rabbit anti-CDK4 (CST; cat no. 12790) diluted at 1:1000 in 5% BSA/TBST was used to blot for CDK4 signal at 4° C. overnight. The next morning the blot was washed 3× with TBST prior to incubation with anti-rabbit IgG HRP conjugated secondary (CST; cat no. 7074) (1:3000 TBST) for 2 h at 22° C. Blots was quickly washed 3× with TBST prior to incubation with ECL western blotting substrates (Promega; cat no. W1001) for 1 min and imaging with ChemiDoc MP.

### Example 22. Compound 148 Decreases Cell Viability and Inhibits Cellular CDK4 Activity

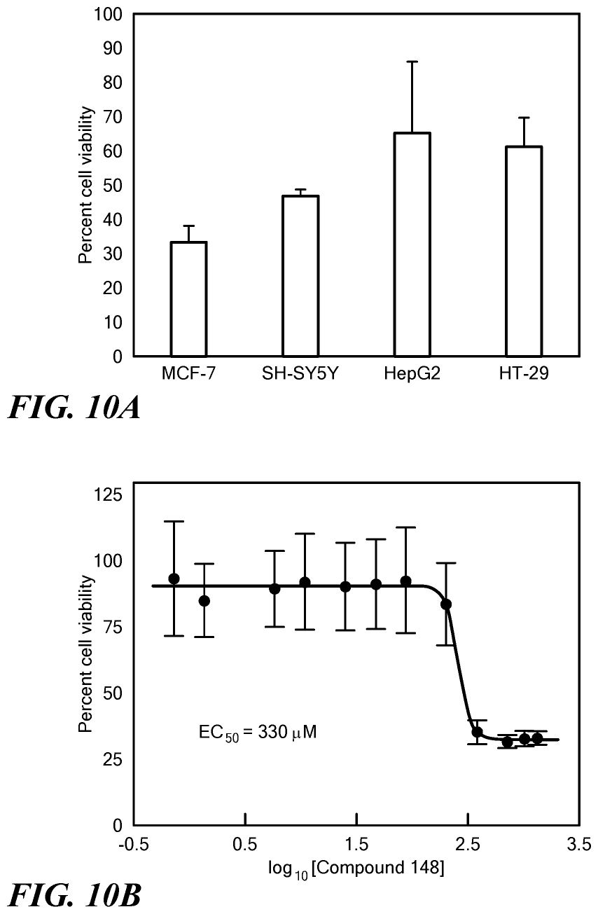

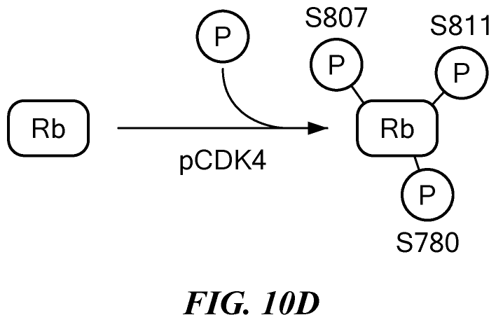

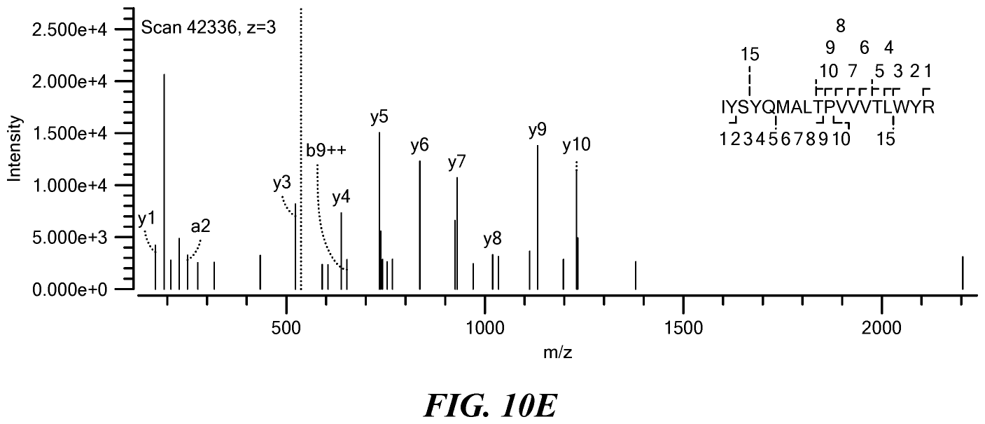

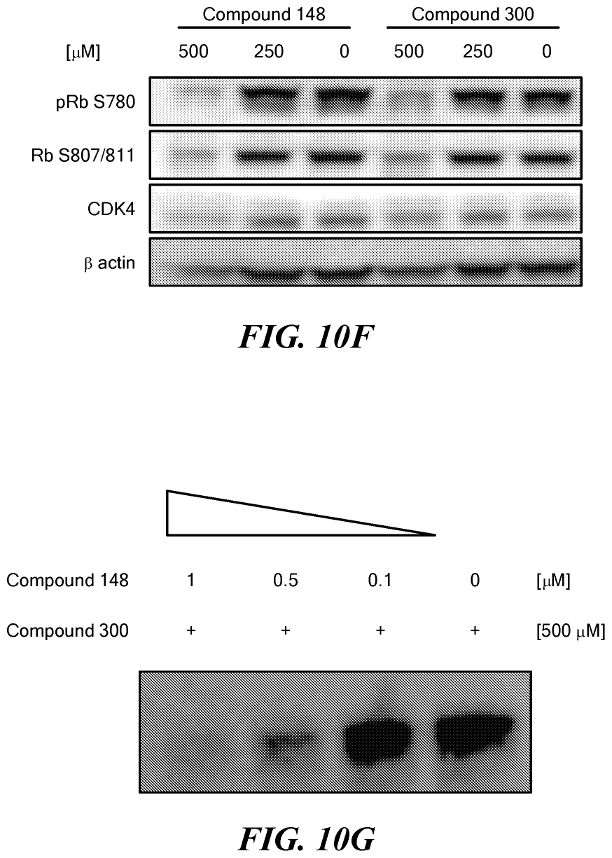

A model cell line that displayed heightened sensitivity to Compound 148 was sought to be identified. Compound 148 was screened across a small panel of cell lines with sensitivity to ribociclib, a clinically-approved CDK4/6 inhibitor, seen in FIG. 10A.

The line most sensitive to Compound 148 treatment, the human breast adenocarcinoma line MCF-7, was selected as a model for further study. A dose-dependent decrease in cell viability of MCF-7 cells in response to Compound 148 was observed, with an EC₅₀ of 330 μM, as seen in FIG. 10B. This lower EC₅₀ observed in cells compared to in vitro is likely the result of several factors including cell permeability, non-productive consumption by thiols, and/or off-target effects.

To further probe how Compound 148 contributes to the observed cellular phenotype, an analogue of Compound 148, Compound 300 was synthesized as in Example 2, containing an alkyne handle for detection and enrichment using click chemistry. Shotgun proteomics experiments, as described in Example 5, revealed that Compound 300 modifies CDK4 selectively at the same M169 site as the parent Compound 148, as shown in FIG. 10E. The effects of both probes on CDK4 activity within a cellular context was tested using western blot analysis, as outlined in Example 18, by monitoring the phosphorylation status of retinoblastoma protein (Rb), the main cellular substrate of CDK4.

When active, CDK4 in complex with its cognate cyclin partner phosphorylates Rb at one of 14 sites, as shown in FIG. 10D.⁸³ Phospho-responsive antibodies specific for three of these sites, S780, S807, and S811, were used as a method to assess CDK4 activity in cellulo.

Indeed, incubation of serum-synchronized MCF-7 cells with either Compound 148 or Compound 300 at a dose of 500 μM resulted in a marked decrease in signal from the pRb antibodies, suggesting a decrease in cellular CDK4 activity under these conditions, as shown in FIG. 10F. CDK4 target engagement in cells was confirmed using a competition binding assay between Compound 148 and Compound 300, as outlined in Example 21, and seen in FIG. 10G.

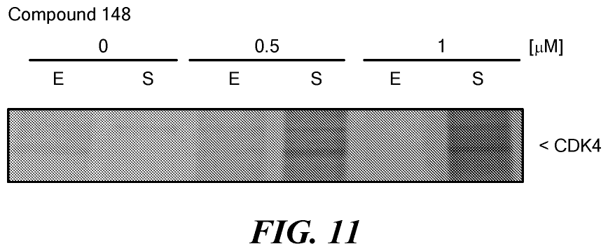

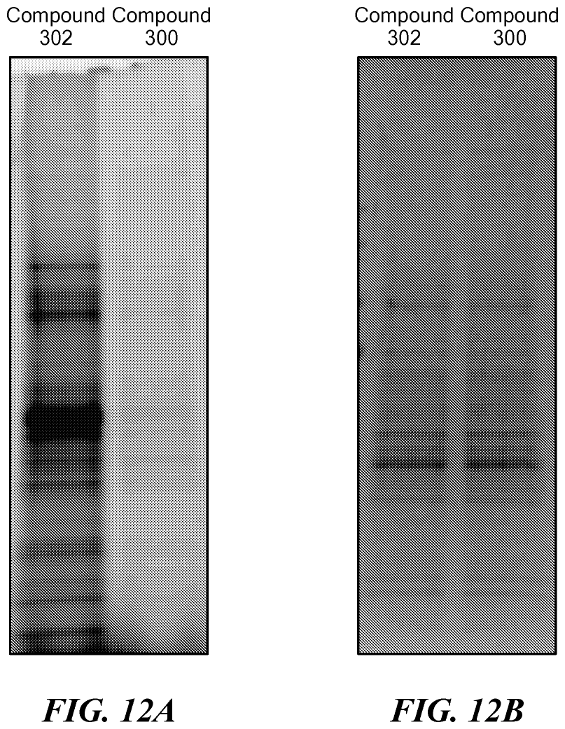

To overcome detection challenges with the low endogenous expression levels of CDK4, even in cells known to upregulate the protein such as MCF-7, transfection was performed to achieve transient overexpression of CDK4 in these models. Treatment of cells with varying amounts of Compound 148 competing ligand prior to incubation with 500 μM Compound 300 showed a dose-dependent decrease in signal in eluted proteins and a corresponding increase in signal in the respective supernatant, indicating competition between the two compounds and engagement with the CDK4 target in a cellular context, seen in FIG. 11. Additionally, Compound 300 displayed lower reactivity in lysate compared to Compound 302, further suggesting its heightened selectivity, seen in FIG. 12.

### Example 23. Biochemical Studies of CDK4 Inhibition by Compound 148 Reveal Reciprocal Crosstalk Between M169 Oxidation and T172 Phosphorylation

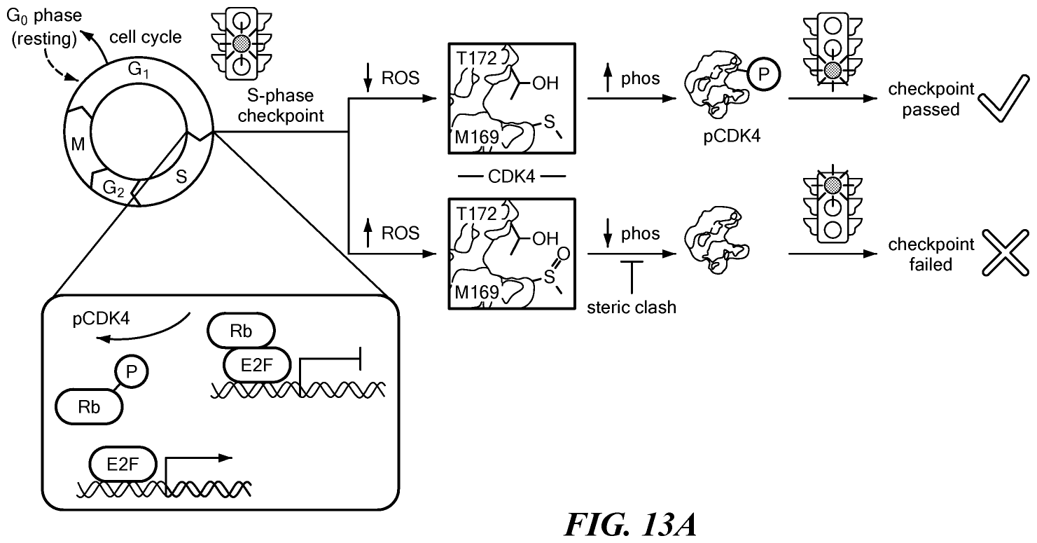

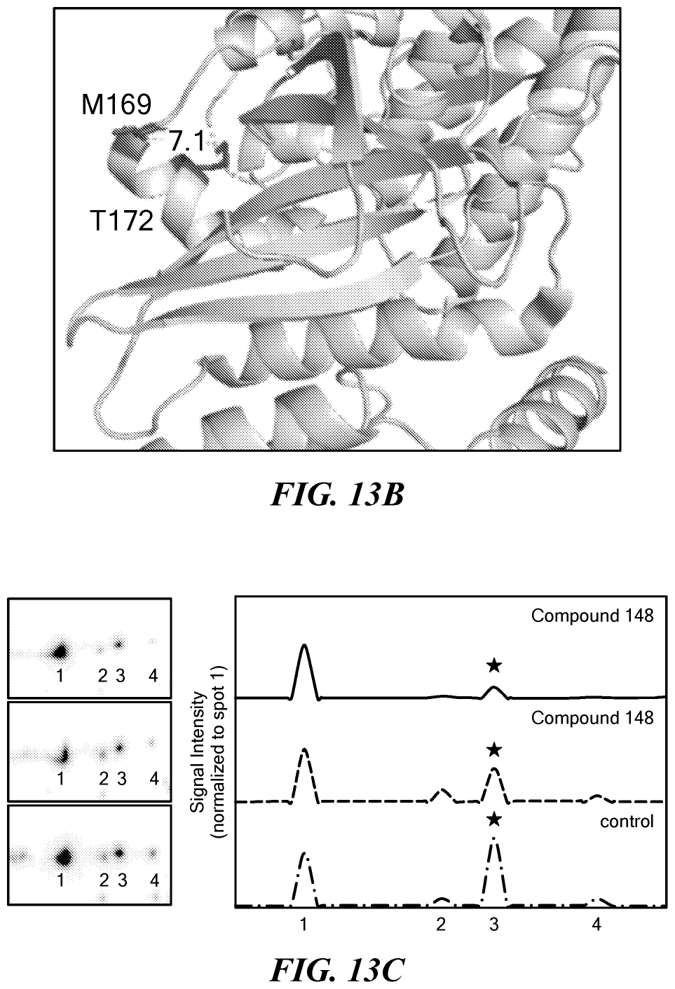

With data establishing loxF11 as a covalent modifier of CDK4 at a newly identified allosteric M169 site, along with its ability to inhibit CDK4 activity on purified protein and in cells and decrease cell viability in a dose-dependent manner, we sought to further interrogate its The potential mechanism of action of Compound 148 on CDK4 was further investigated at the biochemical level. CDK4 plays a key role in the cell cycle in clearing the cell for division, only allowing for passage through the S-phase checkpoint when properly activated. In particular, this signaling pathway relies on proper binding of CDK4 to its respective cyclin, as well as phosphorylation at T172 by cyclin-dependent activating kinase (CAK) to activate the protein. Owing to the proximity of M169 to this activating T172 phosphorylation site, M169 was hypothesized to act as an allosteric redox regulatory switch at this S-phase checkpoint. Oxidation can transform the normally hydrophobic methionine residue into a more hydrophilic and sterically-demanding methionine sulfoxide congener, which could block access of CAK to T172 and prevent its phosphorylation, thus causing the cell to fail the S-phase checkpoint, seen in FIG. 13A. This type of crosstalk between methionine oxidation and adjacent phosphorylation sites has been reported for other systems. Indeed, M169 and T172 lie in a flexible region of CDK4 that can come within 7 angstroms of each other, a distance observed to undergo this phenomenon previously FIG. 13B. Utilizing a custom pCDK4-Thr172 antibody, treatment with Compound 148 was observed to be able to diminish T172 phosphorylation status on CDK4 in MCF-7 cells in a dose-dependent manner, seen in FIG. 13C. Spot 3 corresponds to CDK4 in its T172 phosphorylated state, and spot 1 to unphosphorylated CDK4. Samples were separated via 2D SDS-PAGE, and spot 3 signal was normalized to that of spot 1. These data, along with supporting evidence showing engagement of CDK4 and inhibition of its activity in these same MCF-7 cell models, support a model in which M169 oxidation/T172 phosphorylation crosstalk offers a potential new redox vulnerability where oxidative modification of CDK4 at an allosteric M169 by Compound 148 inhibits CDK4 activity by hindering phosphorylation at its activating T172 site.

### EQUIVALENTS AND SCOPE

This application refers to various issued patents, published patent applications, journal articles, and other publications, all of which are incorporated herein by reference in their entirety. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present disclosure, as defined in the following claims.

1. A compound of Formula (I): 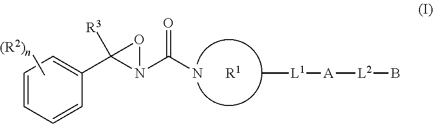
or a pharmaceutically acceptable salt, hydrate, solvate, prodrug, stereoisomer, or tautomer thereof, wherein:
R¹ is an heterocyclyl or heteroaryl, each of which is optionally substituted with one or more R⁴;
R² is C₁-C₆ alkyl, C₂-C₆ alkenyl, C₂-C₆ alkynyl, C₁-C₆ heteroalkyl, C₁-C₆ haloalkyl, halo, cyano, or —OR^{A};
R³ is hydrogen, C₁-C₆ alkyl or halo; each of L¹ and L² is independently absent, C₁-C₆ alkylene, or C₁-C₆ heteroalkylene;
A is absent, cycloalkyl, heterocyclyl, aryl, or heteroaryl, each of which is optionally substituted with one or more R⁵;
B is absent, cycloalkyl, heterocyclyl, aryl, or heteroaryl, each of which is optionally substituted with one or more R⁵;
each of R⁴ and R⁵ is independently C₁-C₆ alkyl, C₂-C₆ alkenyl, C₂-C₆ alkynyl, halo, cyano, or —OR^{A}; or
two of R⁴ or two of R⁵ may come together to form a ring with R¹, A, or B respectively;
R^{A} is hydrogen, C₁-C₆ alkyl, C₂-C₆ alkenyl, C₂-C₆ alkynyl, C₁-C₆ heteroalkyl, C₁-C₆ haloalkyl, cycloalkyl, or heterocyclyl; and
n is 0, 1, 2, 3, 4, or 5;
provided that if both L¹ and A are absent, then L² and B are absent.

2. The compound of claim 1, wherein R¹ is heterocyclyl, optionally substituted with one or more R⁴.

3. The compound of any one of the preceding claims, wherein R¹ is a four-membered heterocyclyl, five-membered heterocyclyl, six-membered heterocyclyl, seven-membered heterocyclyl, or eight-membered heterocyclyl, each of which is optionally substituted with one or more R⁴.

4. The compound of any one of the preceding claims, wherein R¹ is a monocyclic heterocyclyl, optionally substituted with one or more R⁴.

5. The compound of claims 1-3, wherein R¹ is a bicyclic heterocyclyl, optionally substituted with one or more R⁴.

6. The compound of any one of the preceding claims, wherein R² is hydrogen.

7. The compound of any one of the preceding claims, wherein R³ is hydrogen.

8. The compound of any one of the preceding claims, wherein one of L¹ and L² is independently absent.

9. The compound of any one of claims 1-7, wherein of L¹ and L² is independently C₁-C₆ alkylene.

10. The compound of any one of claims 1-7, wherein of L¹ and L² is independently C₁-C₆ heteroalkylene.

11. The compound of any one of the preceding claims, wherein A is absent.

12. The compound of claims 1-10, wherein A is aryl, heteroaryl, cycloalkyl, or heterocyclyl, each of which is optionally substituted with one or more R⁵.

13. The compound of claim 10, wherein A is aryl optionally substituted with one or more R⁵.

14. The compound of claim 10, wherein A is heteroaryl optionally substituted with one or more R⁵.

15. The compound of claim 10, wherein A is cycloalkyl optionally substituted with one or more R⁵.

16. The compound of any one of the preceding claims, wherein B is absent.

17. The compound of claims 1-15, wherein B is aryl, heteroaryl, cycloalkyl, or heterocyclyl, each of which is optionally substituted with one or more R⁵.

18. The compound of claim 17, wherein B is aryl optionally substituted with one or more R⁵.

19. The compound of claim 17, wherein B is heteroaryl optionally substituted with one or more R⁵.

20. The compound of claim 17, wherein B is cycloalkyl optionally substituted with one or more R⁵.

21. The compound of any one of the preceding claims, wherein the compound is a compound listed in any one of Tables 1, 2, or 3.

22. The compound of any one of the preceding claims, wherein the compound of Formula (I) is a compound listed in Table 1.

23. The compound of any one of the preceding claims, wherein the compound is Compound 138 or pharmaceutically acceptable salt, solvate, stereoisomer, or tautomer thereof.

24. The compound of any one of the preceding claims, wherein the compound is Compound 148 or pharmaceutically acceptable salt, solvate, stereoisomer, or tautomer thereof.

25. A compound of Formula (II): 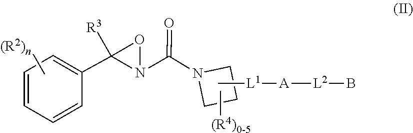
or a pharmaceutically acceptable salt, hydrate, solvate, prodrug, stereoisomer, or tautomer thereof, wherein:
R² is C₁-C₆ alkyl, C₂-C₆ alkenyl, C₂-C₆ alkynyl, C₁-C₆ heteroalkyl, C₁-C₆ haloalkyl, halo, cyano, or —OR^{A};
R³ is hydrogen, C₁-C₆ alkyl or halo; each of L¹ and L² is independently absent, C₁-C₆ alkylene, or C₁-C₆ heteroalkylene;
A is absent, cycloalkyl, heterocyclyl, aryl, or heteroaryl, each of which is optionally substituted with one or more R⁵;
B is absent, cycloalkyl, heterocyclyl, aryl, or heteroaryl, each of which is optionally substituted with one or more R⁵;
each of R⁴ and R⁵ is independently C₁-C₆ alkyl, C₂-C₆ alkenyl, C₂-C₆ alkynyl, halo, cyano, —OR^{A}, or
two of R⁴ may come together to form a ring bound to the azetidinyl ring, or wherein or two of R⁵ may come together to form a ring with A or B respectively;
R^{A} is hydrogen, C₁-C₆ alkyl, C₂-C₆ alkenyl, C₂-C₆ alkynyl, C₁-C₆ heteroalkyl, C₁-C₆ haloalkyl, cycloalkyl, or heterocyclyl; and
n is 0, 1, 2, 3, 4, or 5; provided that if both L¹ and A are absent, then L² and B are absent.

26. The compound of any one of the preceding claims, wherein the compound of Formula (II) a compound listed in any one of Tables 1, 2, or 3.

27. The compound of any one of the preceding claims, wherein the compound of Formula (II) is a compound listed in Table 2.

28. A compound of Formula (III): 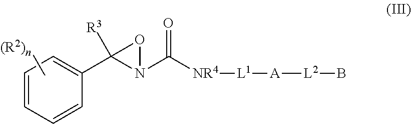
or a pharmaceutically acceptable salt, hydrate, solvate, prodrug, stereoisomer, or tautomer thereof, wherein:
R² is C₁-C₆ alkyl, C₂-C₆ alkenyl, C₂-C₆ alkynyl, C₁-C₆ heteroalkyl, C₁-C₆ haloalkyl, halo, cyano, or —OR^{A};
R³ is hydrogen, C₁-C₆ alkyl or halo; each of L¹ and L² is independently absent, C₁-C₆ alkylene, or C₁-C₆ heteroalkylene;
A is absent, cycloalkyl, heterocyclyl, aryl, or heteroaryl, each of which is optionally substituted with one or more R⁵;
B is absent, cycloalkyl, heterocyclyl, aryl, or heteroaryl, each of which is optionally substituted with one or more R⁵;
R⁴ is independently hydrogen, C₁-C₆ alkyl, or cycloalkyl;
each R⁵ is independently C₁-C₆ alkyl, C₂-C₆ alkenyl, C₂-C₆ alkynyl, halo, cyano, —OR^{A}, or
two of R⁵ may come together to form a ring with A or B respectively;
R^{A} is hydrogen, C₁-C₆ alkyl, C₂-C₆ alkenyl, C₂-C₆ alkynyl, C₁-C₆ heteroalkyl, C₁-C₆ haloalkyl, cycloalkyl, or heterocyclyl; and
n is 0, 1, 2, 3, 4, or 5; provided that if both L¹ and A are absent, then L² and B are absent.

29. The compound of any one of the preceding claims, wherein the compound of Formula (III) a compound listed in any one of Tables 1, 2, or 3.

30. The compound of any one of the preceding claims, wherein the compound of Formula (III) is a compound listed in Table 3.

31. A composition comprising a compound described herein, e.g., a compound of Formula (I), (II), (III), or pharmaceutically acceptable salt, solvate, stereoisomer, or tautomer thereof according to any one of the preceding claims, and one or more pharmaceutically acceptable carriers.

32. A method of covalently labeling a methionine residue in a target protein or a target peptide, the method comprising contacting the target protein or target peptide with a compound of Formula (I), (II), or (III) described herein.

33. The method of claim 32, wherein the target protein is a kinase.

34. The method of any one of claims 32-33, wherein the target protein is a cyclin-dependent kinase.

35. The method of any one of claims 33-34, wherein the target protein is CDK4.

36. An activity-based protein profiling (ABPP) method comprising activity-based profiling of a target protein using Redox-Activated Chemical Tagging (ReACT) for bioconjugation by targeting a methionine of the protein through the use of oxaziridine reagents that promote selective nitrene fragment transfer reactivity that is isoelectronic to native methionine oxidation by oxygen atom transfer.

37. The method of claim 36, wherein the target protein is CDK4.

38. An N-transfer oxidant compound of Formula (I-a): 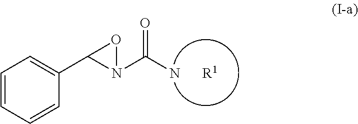
wherein R1 is an optionally substituted 5- to 14-membered heteropolycycle.

39. The compound of claim 38, wherein R1 is an optionally substituted spirocycle, fused heterocycle, bridged heterocycle, or combination thereof.

40. The compound of claim 38 or 39 wherein R1 comprises a 4, 5, 6 or 7 membered first ring fused, bridged or linked by one or more common atoms to a second ring.

41. The compound of claim 40, wherein the first ring is saturated and comprises 0, 1 or 2 heteroatoms (e.g. N or O) in addition to the N shown (e.g. azetidinyl, pyrrolidinyl, pipiridinyl, azepanyl, diazinanyl, morpholinyl).

42. The compound of claim 40 or 41, wherein the second ring is 3, 4, 5 or 6 membered, saturated or unsaturated, optionally comprising 1-3 heteroatoms (e.g. N or O).

43. The compound of claim 38, having a structure of Table 1.

44. The compound of claim 38, having a structure of Table 2.

45. The compound of claim 38, having a structure of Table 3.

46. A method of chemoselective conjugation comprising reacting the N-transfer oxidant compound of any one of claims 38-45, with a thioether substrate in an aqueous environment to form a conjugation product comprising a resultant sulfimide.
